(19) United States
(12) Patent Application Publication
(10) Pub. No.: US 2022/0282284 A1
(43) Pub. Date: Sep. 8, 2022

(54) CRISPR GENOME EDITING WITH CELL SURFACE DISPLAY TO PRODUCE HOMOZYGOUSLY EDITED EUKARYOTIC CELLS

(71) Applicant: The Rockefeller University, New York, NY (US)

(72) Inventors: Sebastian KLINGE, New York, NY (US); Sameer Kumar SINGH, New York, NY (US)

(21) Appl. No.: 17/635,358

(22) PCT Filed: Aug. 14, 2020
(86) PCT No.: PCT/US2020/046478

§ 371 Date: Feb. 14, 2022

Related U.S. Application Data
(60) Provisional application No. 62/887,172, filed on Aug. 15, 2019.

(51) Int. Cl. C12N 15/90; C12N 15/10; C12N 9/22

(52) U.S. Cl. CPC C12N 15/907; C12N 15/1037; C12N 9/22; C12N 2310/20; C12N 2800/30; C12N 15/85

(57) ABSTRACT

Provided are compositions and methods for producing eukaryotic cells that comprise homozygous modifications. The modifications include homozygous insertions of a modified open reading frame (a “mORF”), and removable surface displayed epitopes that can be used for separating cells that contain the homozygous modifications by Fluorescence-activated cell sorting (FACS). The inserted mORFs are configured so that they are in frame with an endogenous open reading frame and their expression can be controlled by an endogenous promoter. The homozygous insertions are produced using specialized double stranded DNA repair templates and CRISPR-based approaches, which provide for insertion of the homozygous modified ORFs, surface expression of two different epitopes that are separated from the modified ORFs by ribosomal peptide skipping domains, and separation and isolation of cells that contain the homozygous insertions, with concurrent or sequential removal of the epitopes using recombinase-mediated approaches. Cells made using the compositions and methods are also provided.

### CROSS REFERENCE TO RELATED APPLICATIONS

This application claims priority to U.S. provisional application No. 62/887,172, filed Aug. 15, 2019, the entire disclosure of which is incorporated herein by reference.

### FIELD

The present disclosure relates to modified eukaryotic cells, and methods for making the modified eukaryotic cells. The eukaryotic cells comprise homozygous insertions.

### BACKGROUND

There is an ongoing and unmet need for improved compositions and methods for generating eukaryotic cells that comprise homozygous modifications of a particular chromosomal locus. The present disclosure is pertinent to this need.

### SUMMARY

The present disclosure provides new and improved compositions and methods for producing eukaryotic cells that comprise homozygous modifications. The modifications include, among other components, homozygous insertions of a modified open reading frame (a “mORF”), and removable surface displayed epitopes that can be used for separating cells that contain the homozygous modifications, such as by Fluorescence-activated cell sorting (FACS). The inserted mORFs can be introduced such that they are in frame with an endogenous open reading frame. As such, expression of the inserted mORFs can be controlled by an endogenous promoter. The insertions can be in any segment of a gene that contains an open reading frame, e.g., in any exon. In embodiments, the insertions are in the last exon of a gene, at least in part to facilitate sorting by the separate surface exposed, removable epitopes. The disclosure includes cells made by the described method, which may be any eukaryotic cell types.

Accordingly, in one aspect, the disclosure provides a method for producing a population of eukaryotic cells comprising a homozygous insertion of first and second DNA segments into a chromosomal locus. The method comprises introducing into the cells a first and second double stranded (ds) DNA repair template, each of which is optionally provided as a component of a plasmid. The first dsDNA repair template comprises a 5′ homology segment which contains a dsDNA sequence for integration into a chromosome sequence that is homologous to the 5′ homology segment, and 3′ homology segment that contains a dsDNA sequence for integration into a chromosome sequence that is homologous to the 3′ homology segment. The first and second dsDNA repair templates comprise the mORF, and also comprise a sequence encoding a ribosomal peptide skipping domain, a sequence encoding a secretion signal; a sequence encoding a first epitope that can be recognized with specificity by a detectably labeled first antibody, optionally a sequence encoding a linker, and a sequence encoding a transmembrane domain (TMD). These components may be provided sequentially in a 5′ to 3′ orientation. The second dsDNA repair template is the same as the first, with the exception that the second dsDNA repair template contains a sequence encoding a second epitope that is different from the first, that can be recognized with specificity by a detectably labeled second antibody that is different from the first detectably labeled antibody. Accordingly, the first and second antibodies are labeled with different detectable labels.

Along with the first and second dsDNA repair templates, the method comprise introducing into the cells a Clustered Regularly Interspaced Short Palindromic Repeats (CRISPR) associated protein, e.g., a Cas enzyme, or a polynucleotide encoding the Cas enzyme. While embodiments of the disclosure are demonstrated using Cas9, other Cas enzymes that will be recognized by those skilled in the art can be used, provided they are accompanied by a suitable guide RNA. The disclosure also includes introducing the Cas enzyme and the guide RNA by using expression vectors encoding these components, or mRNA encoding these components, or a by using a complex of proteins and RNA, such as a ribonucleoprotein (RNP). The guide RNA comprises a sequence that recognizes a protospacer in the chromosome such that a complex comprising the Cas enzyme and the guide RNA can facilitate homologous recombination of the first and second dsDNA repair templates into a first and second allele of the same chromosomal locus, thereby providing a eukaryotic cell comprising a homozygous replacement of the first and second alleles with the first and second dsDNA repair templates. Expression of the first allele results in expression of the first epitope, and expression of the second allele results in expression of the second epitope. More than one of each epitope can be included.

In certain embodiments, the mORF comprises a sequence encoding a corrected version of an ORF that contains one or more deleterious mutations, a protein that produces a fluorescent signal, or a sequence used for purification of the protein.

In certain embodiments, constructs of the disclosure are configured such that the first and second dsDNA repair templates comprise sequences encoding recombinase recognition sequences. The recombinase recognition sequences flank at least the first and second epitope sequences. The recombinase recognition sequences are operative with a recombinase that can excise chromosomal segments comprising the first and second epitopes. The disclosure therefore also includes expressing a recombinase that recognizes the recombinase recognition sequences in the cells, such that the recombinase excises at least the first and second epitopes, but leaving the sequence encoding the mORF in the first and second alleles.

The disclosure also includes methods for producing a population of single cell clones that contain a homozygous chromosomal insertion by using the described method, and separating the cells that express the first and second epitopes from cells that do not express the first and second epitopes. In this regard, it is considered that the described method is more efficient than previously available approaches, insofar as at least 10% of the cells separated from the population into which the first and second dsDNA repair templates, the Cas enzyme, and the guide RNA are introduced comprise the homozygous chromosomal insertion. The disclosure provides demonstrations wherein at least 35% of the cells separated from the population into which the first and second dsDNA repair templates, the Cas enzyme, and the guide RNA are introduced comprise the homozygous chromosomal insertion. The disclosure includes single cells, and populations of cells, that are made by the described method. The disclosure also includes kits for producing eukaryotic cells that contain homozygous insertions.

### BRIEF DESCRIPTION OF THE DRAWINGS

FIG. 1. Schematic representation of SNEAK PEEC. (A) schematic representation of DNA repair templates with homology arms, a tagged gene of interest, P2A site, secretion signal (SS), epitopes and a transmembrane domain (TMD). (B) Schematic representation of outcomes after transfection highlighting the presence of epitopes 1 and 2 with indicated genotype for the tagged gene. The addition of labelled epitope-specific dyes (C) precedes fluorescence activated cell sorting (FACS; D) and PCR verification (E).

FIG. 2. Additional embodiment of SNEAK PEEC. (A) Introduction of recombination sites (loxP, FRT or lox variants) within a DNA repair template containing a C-terminal tag and a surface epitope (epitope N; top) and its product following recombination (bottom). (B) N-terminal tagging design for SNEAK PEEC including recombination sites as in (A) with a product following recombination. (C) Signal amplification for lowly expressed genes by using peptide epitope arrays of different amino acid sequences.

FIG. 3. Representative embodiment of a general DNA repair template used in SNEAK PEEC. Schematic illustration of a DNA repair template containing homology regions for targeting to the gene of interest (5′ and 3′ homology). The gene of interest is then followed by a 3C protease cleavable linker and a GFP tag. This tag is followed by a 2A viral peptide (P2A, T2A, E2A or the like) that generates the downstream segment as a physically separate polypeptide. A secretion signal is followed by one of several surface epitopes (epitope 1, epitope 2, epitope 3 etc.) that is displayed on the cell surface via a dedicated transmembrane domain (TMD). The transcript also contains a polyadenylation signal as indicated. A PacI site after the polyadenylation signal marks the 3′ end of the inserted DNA before the 3′ homology. Sites for restriction endonucleases are indicated on the top. The introduction of specific DNA sequences (FRT, loxP, sgRNA) flanking the surface epitope cassette enables the removal of these elements to allow for iterative genome editing.

FIG. 4. Rows 1 -2: Live, single cells were first isolated from a starting population of approximately 150,000 cells, based on their dead cell exclusion as well as forward and side scatter profiles (FSC, SSC). Rows 3 -4: Live GFP and mCherry positive cells were then selected (DP). Of these, cells positive for both anti-STAS Janelia646 and anti-porM_APC-Cy7 were selected (P1). A total of 143 cells were selected in this manner.

FIG. 5. PCR validation of homozygously edited single cell clones: PCR primers flanking the STAS and porM DNA were used to detect homozygotes. (A) For the 2× DNA experiment 11/29 clones (38%) are positive for both STAS and porM DNA (homozygotes, denoted as &). (B) For the 1× DNA experiment 8/20 clones (40%) are positive for both STAS and porM DNA (homozygotes, denoted as &).

FIG. 6. PCR validation of complete and site-specific genomic integration: PCR validation was carried out using a forward primer (Fwd) flanking the left homology arm of the repair template, binding DNA in the unedited genomic DNA sequence. The reverse primer (Rev) binds specifically to either the STAS or porM sequence.

FIG. 7. Surface display inactivation via sgRNA (sgRNA expressing plasmid transfected in Opti-MEM medium). Rows 1 -2: Live, single cells were first isolated from a starting population of approximately 58,500 cells, based on their dead cell exclusion as well as forward and side scatter profiles (FSC, SSC). Rows 3 -4: Live GFP and mCherry positive cells were then selected (DP). Of these, cells negative for both anti-STAS_Janelia646 and anti-porM_APC-Cy7 were selected (P1). A total of 96 clones were selected in this manner.

FIG. 8. Surface display inactivation via sgRNA (sgRNA expressing plasmid transfected in GIBCO Freestyle 293 medium). Rows 1 -2: Live, single cells were first isolated from a starting population of approximately 66,000 cells, based on their dead cell exclusion as well as forward and side scatter profiles (F SC, SSC). Rows 3 -4: Live GFP and mCherry positive cells were then selected (DP). Of these, cells negative for both anti-STAS_Janelia646 and anti-porM_APC-Cy7 were selected (P1). A total of 96 clones were selected in this manner.

FIG. 9. PCR amplifications on samples demonstrating insertion of porM and STAS domain coding sequences into genome. Two PCR amplifications were performed for each sample.

FIG. 10. PCR amplifications demonstrating verification of identified single cell homozygous clones from a direct sort from transfected 293-F cells.

FIG. 11. Representative schematic demonstrating a workflow for recombinase-mediated removal of cell surface epitope that can be performed based on the disclosure. A. DNA repair templates 1 and 2 for transfection into cells. B. Second transfection with inducible recombinase and reporter. C. Induction of recombinase shortly before cell sorting to facilitate sorting while surface epitopes still present. D. Epitope specific dyes. E. FACS sorting. F. PCR verification of separation of cells containing tagged (modified ORF) and cells that do not contain modified ORF.

FIG. 12. Schematic and data showing transfection and cell sorting as used in SNEAK PEEC display epitope recycling. A display removal plasmid encoding Flp recombinase and BFP was transfected into a clonal population of a homozygously edited clone (Noc4l-gfp-Display Hivp24/Btuf). FACS sorting was used to select cells positive for mCherry, GFP and Bfp.

FIG. 13. Schematics and PCR products illustrating genotyping confirmation of removal of display epitope by genotyping sorted single cell clones.

FIG. 14. Schematics and PCR products illustrating further confirmation genotyping shown removal of display epitope and retention of inserted ORF.

FIG. 15. Construct for use in peptide epitope arrays as display epitopes with ribosome skipping sequence.

FIG. 16. Workflow showing SNEAK PEEC for use in selected cells in which the WDR12 gene has been homozygously edited. Data show 7/8 (87.5%) of sorted cells contain a homozygous insertion.

### DETAILED DESCRIPTION OF THE DISCLOSURE

Unless defined otherwise herein, all technical and scientific terms used in this disclosure have the same meaning as commonly understood by one of ordinary skill in the art to which this disclosure pertains.

Every numerical range given throughout this specification includes its upper and lower values, as well as every narrower numerical range that falls within it, as if such narrower numerical ranges were all expressly written herein. All time intervals, temperatures, reagents, culture conditions and media, methods of detecting and isolating cells, isolated cells, purified cells, single cell clones, and populations of isolated single cell clones described herein are included in this disclosure. This disclosure includes all nucleic acid and amino acid sequences described herein and all contiguous segments thereof. The disclosure includes all polynucleotide sequences, their RNA or DNA equivalents, all complementary sequences, and all reverse complementary sequences. If reference to a database entry is made for a sequence, the sequence is incorporated herein by reference as it exists in the database as of the filing date of this application or patent. Any reference to a database entry for an amino acid and/or polynucleotide sequence includes incorporation of said sequence herein by reference, as said sequence is shown in the database as of the filing date of this application or patent. The disclosure of all patents and patent publications referenced in this disclosure are incorporated herein by reference. The disclosure includes sequences that are from 80.0% to 99.9% identical to said sequences across their entire lengths. The disclosure includes all polypeptide sequences encoded by nucleotide sequences presented in this disclosure.

The disclosure includes all steps and compositions of matter described herein in the text and figures of this disclosure, including all such steps individually and in all combinations thereof, and includes all compositions of matter including but not necessarily limited to vectors, cloning intermediates, cells, cell cultures, progeny of the cells, and the like. The disclosure includes cells that are in culture, and are in flow, such as during cell sorting, and includes all progeny of the cells, whether or not such cells or their progeny are introduced into an animal.

Throughout this application, unless stated differently, the singular form encompasses the plural and vice versa. All sections of this application, including any supplementary sections or figures, are fully a part of this application.

The term “treatment” as used herein refers to alleviation of one or more symptoms or features associated with the presence of the particular condition or suspected condition being treated. Treatment does not necessarily mean complete cure or remission, nor does it preclude recurrence or relapses. Treatment can be effected over a short term, over a medium term, or can be a long-term treatment, such as, within the context of a maintenance therapy. Treatment can be continuous or intermittent.

The term “therapeutically effective amount” as used herein refers to an amount of an agent sufficient to achieve, in a single or multiple doses, the intended purpose of treatment. The amount desired or required will vary depending on the particular compound or composition used, its mode of administration, patient specifics and the like. Appropriate effective amounts can be determined by one of ordinary skill in the art informed by the instant disclosure using routine experimentation.

This disclosure provides modified eukaryotic cells, vectors and cells comprising nucleic acids encoding a modified chromosomal sequence, compositions comprising any of the foregoing, methods of making any of the foregoing, and methods of using the modified eukaryotic cells for any purpose, non-limiting examples of which include providing modified cells for use in the study or any particular cellular function or protein attribute, protein expression profile, intracellular location, or other uses that will be apparent from the present disclosure. The disclosure includes all modified cells as they exist during separation, such as during any form of cell cytometry, FACS, and the like, and as they exist post-separation from other, non-modified cells. The disclosure includes treatment and/or prophylaxis of a condition associated with a condition that is associated with unmodified alleles, wherein a modified homozygous pair of alleles are introduced into chromosomes such that the modified sequence is homozygous, and provides a therapeutic and/or prophylactic benefit to a recipient of the modified cells.

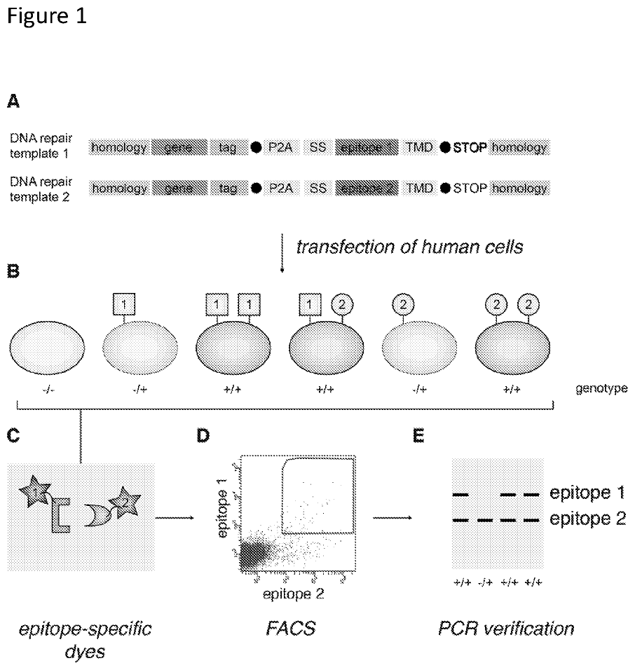

In more detail, the present disclosure provides a method that is referred to as Surface engiNeered fluorEscence Assisted Kit with Protein Epitope Enhanced Capture (SNEAK PEEC), an approach that combines CRISPR/Cas genome editing with cell-surface display to isolate homozygously edited eukaryotic cells. In embodiments, eukaryotic cells are transfected with two DNA repair templates that target the two alleles of the same gene. These two DNA repair templates can for example contain an identical tag downstream of the gene of interest or any other gene modification, which is followed by a viral peptide ribosome skipping sequence that physically separates the subsequent protein coding segment from the gene of interest. Downstream of the viral peptide a secretion signal then precedes two different epitopes (epitope 1 or epitope 2) in the two different DNA repair templates, which are exposed on the cell surface via a transmembrane domain (see, for example, FIG. 1A).

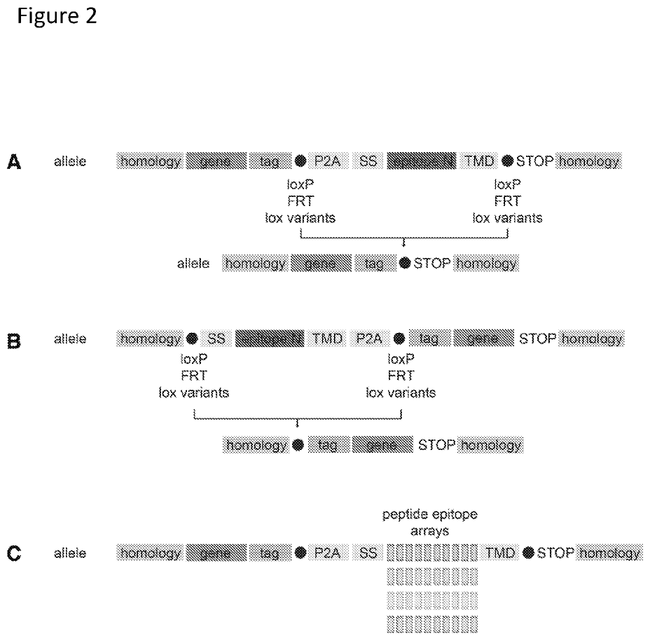

Only correct in-frame insertions of these DNA templates will generate cell surface epitopes and additionally the entire topology of this system can also be inverted to allow for N-terminal tagging with epitopes upstream of a gene of interest (FIG. 2) or for homozygous gene knockouts. A transfection of human cells with both DNA repair templates and Cas9 can therefore result in six different outcomes of cells either containing no edited gene or different heterozygous (−/+) or homozygous (+/+) outcomes. Of these outcomes only one includes both epitopes on the cell surface, which represents a homozygously edited clone (FIG. 1B). The addition of labelled antibodies that are specific for the two epitopes (FIG. 1C) then allows for fluorescence-assisted cell sorting (FACS) to identify and select single homozygous clones containing both epitopes on the cell surface (FIG. 1D). These cells are subsequently verified by PCR for the presence of both epitopes (FIG. 1E). Another round of genome editing can then be performed using different epitopes. Experiments in transfected cell lines show that this system greatly enhances the speed and efficiency of genome editing, since at least approximately 30% of obtained clones are homozygous with generous selection during FACS. Compared with current techniques for which frequently more than 100 clones have to be tested to identify a homozygous knock-in, providing the present disclosure with previously unavailable advantages, such as because the number of clones that need to be screened is much smaller.

An aspect of iterative genome editing using SNEAK PEEC is a set of two orthogonal surface epitope pairs and their removal from edited cells so that recycling of these epitopes can be employed. The introduction of specific DNA recombination sites flanking the surface epitope will allow for the removal of the epitope tags by DNA recombinases whether these are located upstream or downstream of a gene of interest (FIG. 2A, B). By using different DNA recombination sites for different gene editing events, iterative genome engineering will be possible. To further enhance the robustness of SNEAK PEEC, the disclosure provides surface peptide epitope arrays (FIG. 2C) such as repeats of commonly used epitopes (10×FLAG, 10×HA, 10×V5, 10×PA, etc.), which will amplify the surface signal for lowly expressed genes. Iterative genome editing using SNEAK PEEC will facilitate sequential homozygous editing, as also described in the figures of this disclosure.

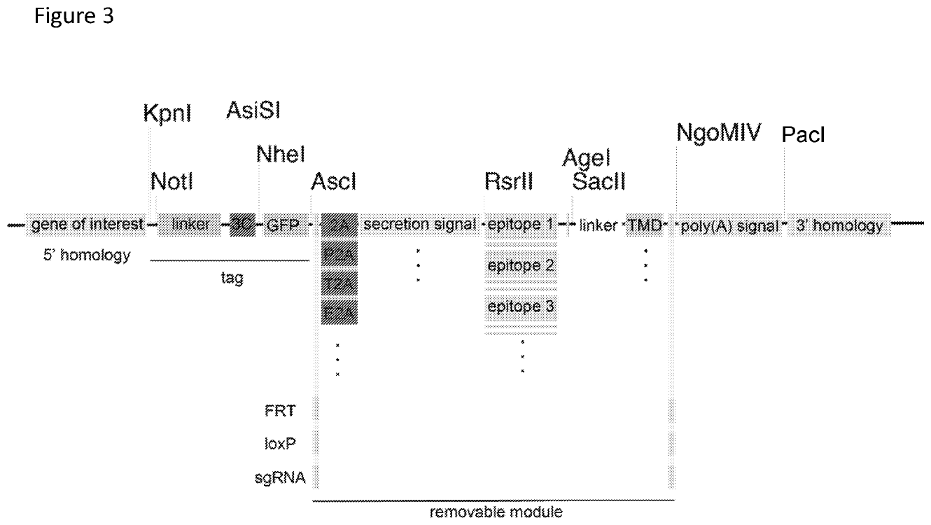

A non-limiting general description of DNA elements used for SNEAK PEEC is presented in FIG. 3.

In embodiments, the disclosure includes use of linker sequences. The linker is typically three amino acids long, and may include a GSG sequence, but other sequences may be used. In embodiments, the linker is from 3-100 amino acids in length. In embodiments, the linker is from 4-40 amino acids. In embodiments, the linker comprises or consists of SGSG (SEQ ID NO:1), GASGSG (SEQ ID NO:2), GGTGSGGSAGGTGGSAGGSAGAGGATGGSTAGGATTAS (SEQ ID NO:3), SNSADGDGSNATGSSAGAGSGTSGGDNTSDGSGASAGAASTNSNGNTGSATSGGAT GSDTSGATAGSGASDGGNGATASSTTGNGNSSGTTATTGGGDAG (SEQ ID NO:4), and including any segment thereof that is at least three amino acids long.

In embodiments, the disclosure includes use of one or more transmembrane domains (TMDs), which are used to anchor proteins comprising epitopes as described herein to cell surfaces. In embodiments, the proteins are not displayed on the cell surface via a sugar molecule, including but not limited to a phosphorylated sugar, such as glycophosphatidylinositol (GPI). In embodiments, a protein epitope anchor of this disclosure does not include CD52. Suitable transmembrane domains include, but are not limited to: a member of the tumor necrosis factor receptor superfamily, CD30, platelet derived growth factor receptor (PDGFR, e.g. amino acids 514-562 of human PDGFR; Chestnut et al., 1996, J Immunological Methods, 193:17-27; also see Gronwald et al., 1988, PNAS, 85:3435-3439); nerve growth factor receptor, Murine B7-1 (Freeman et al., 1991, J Exp Med 174:625-631), asialoglycoprotein receptor H1 subunit (ASGPR; Speiss et al. 1985 J Biol Chem 260:1979-1982), CD27, CD40, CD120a, CD120b, CD80 (B7) (Freeman et al., 1989, J Immunol, 143:2714-2272) lymphotoxin beta receptor, galactosyltransferase (e.g., GenBank accession number AF155582), sialyltransferase (E.G. GenBank accession number NM_003032), aspartyl transferase 1 (Asp1; e.g. GenBank accession number AF200342), aspartyl transferase 2 (Asp2; e.g. GenBank accession number NM_012104), syntaxin 6 (e.g. GenBank accession number NM-005819), ubiquitin, dopamine receptor, insulin B chain, acetylglucosaminyl transferase (e.g. GenBank accession number NM_002406), APP (e.g. GenBank accession number A33292), a G-protein coupled receptor, thrombomodulin (Suzuki et al., 1987, EMBO J, 6:1891-1897) and TRAIL receptor.

In embodiments, the disclosure provides a substantially pure, or completely pure, population of single cells that each comprise the same homozygous insertion. Thus, in embodiments, the disclosure does not provide a polyclonal population of cells.

The disclosure also includes ribosomal skipping sequences, which are also referred to in the art as “self-cleaving” amino acid sequences. These are typically about 18-22 amino acids long. Any suitable sequence can be used, non-limiting example of which include T2A, comprising the amino acid sequence: EGRGSLLTCGDVEENPGP (SEQ ID NO:5); P2A, comprising the amino acid sequence ATNFSLLKQAGDVEENPGP (SEQ ID NO: 6); E2A, comprising the amino acid sequence QCTNYALLKLAGDVESNPGP (SEQ ID NO: 7); and F2A, comprising the amino acid sequence VKQTLNFDLLKLAGDVESNPGP (SEQ ID NO: 8).

In embodiments, and as discussed above, the disclosure comprises introducing into eukaryotic cells two double stranded (ds) DNA repair templates. The dsDNA repair templates comprise first and second homology arms (e.g., 5′ and 3′ homology segments) which are configured to be introduced into desired homozygous chromosomal loci. In embodiments, the first and second homology arms may or may not comprise PCR donor molecules. In embodiments, the first and second homology arms, as well as other components of the system as described and illustrated herein, are provided as a component of one or more plasmids. The sequence of the 5′ and 3′ homology segments are not particularly limited, provided they have a length that is adequate for homologous recombination to occur when Cas-mediated cleavage of the target loci in homozygous alleles is performed. In embodiments, the 5′ and 3′ homology segments have a length of from 50-600 bp, inclusive, and including all integers and ranges of integers there between. The first and second homology arms can include sequences that are recognized and cleaved by the same Cas-mediated cleavage system that recognizes and cleaves the chromosomes, as described and illustrated further herein. The Cas cleavage sites may be positioned at or near the end of the homology arms. This configuration is particularly useful when, for example, the dsDNA repair templates are provided on one or two plasmids. Thus, excision of the plasmid-based DNA repair template facilitates the liberation of the homology ends to aid in homologous recombination into the chromosomes. The genes into which the dsDNA repair templates are introduced is not particularly limited provided sufficient homology is present in the 5′ and 3′ segments. Representative and non-limiting examples of insertions and insertion targets are provided herein in the examples and figures.

In embodiments, the dsDNA repair templates are designed to replace an open reading frame such that two alleles at the same locus are made to be homozygous. In embodiments, the dsDNA repair templates include what may be described herein for convenience as a “tag” but includes a comprises a modified open reading frame (ORF), the modified ORF referred to herein as “mORF.” The mORF comprises a difference in nucleotide sequence, relative to the sequence of one or both alleles in the chromosome prior to performing a method of this disclosure. In this regard, the term “tag” when referring to a mORF as used herein may be different from a tag conventionally used solely for isolation and/or purification of proteins, which may be referred to as purification tags. Thus, the purification tag in embodiments comprises a protein sequence that can be used for affinity purification of a protein of interest. Suitable purification tags are known in the art and can be adapted for use in the compositions and methods of this disclosure, non-limiting examples of which is a His or similar tag, and any epitope for antibody or nanobody-based purification (FLAG, HA, MYC, etc.).

In embodiments, the mORF comprises a single nucleotide change relative to the endogenous ORF. In embodiments, the mORF comprises a more than one nucleotide change relative to the endogenous ORF. In embodiments, the mORF comprises a full new sequence that was not present in the alleles prior to being modified as described herein. In embodiments, the mORF is comprised by sequence which corrects an ORF in one or both alleles in a single locus in a chromosome. In embodiments, the mORF comprises a protein that can produce a detectable signal, such as a fluorescent protein. In embodiments, the signal produced by the protein is distinct from the signal from antibodies that are used to separate cells that have been homozygously modified as described herein. In embodiments, the mORF encodes a segment of a protein that is produced as a fusion protein. In embodiments, a contiguous sequence comprising the mORF is inserted into the last exon of a gene. In embodiments, the mORF is configured such that its open reading frame is inserted into the last exon of a gene such that the mORF is in frame with the preceding exon in a spliced mRNA transcribed from the gene. Thus, the mORF need not include a codon for an initiating methionine. In embodiments, the dsDNA templates are inserted into a locus such that expression of coding sequences comprised by the dsDNA templates is controlled by an endogenous promoter. An “endogenous” promoter is a promoter that is operatively linked to the gene into which the dsDNA sequence is introduced and was present in said operative linkage with the gene prior to insertion of the dsDNA templates. Thus, in embodiments, the dsDNA templates may be free of any promoter that is operably linked to the mORF, and wherein said promoter is operable in the cell into which the dsDNA templates are introduced.

In embodiments, the first and second homology arms are homologous to an allele that encodes or is in tight or complete linkage disequilibrium with an ORF. In embodiments, mORF encodes a protein that is associated with a cellular phenotype. In embodiments, the cellular protein is associated with compartmentalization, which is a key process used to concentrate, organize, and separate macromolecules in distinct subcellular regions.

In embodiments, each dsDNA repair template encodes a distinct epitope. The amino acid sequences of the epitopes are not particularly limited, provided they can each be separately recognized by any suitable binding partner(s). In embodiments, the epitopes may be present in a sequence that is from about 6-1000 amino acids in length. In embodiments, short epitopes may be used, non-limiting examples of which include about 6-20 amino acids for short peptide epitopes such as FLAG, HA, MYC, V5, or PA. In embodiments, the epitopes may be repeated. Repeating the epitopes provides a plurality of binding partner binding sites, which enables amplification of the signal produced by the labelled binding partners. This approach is particularly suited for identifying cells comprising homozygous insertions, such as within genes that are expressed at low levels. Representative and non-limiting epitopes and antibodies used for cell sorting are described herein by way of the figures and examples. In non-limiting embodiments, the following combinations of epitopes and antibodies are use: porM/STAS and corresponding nanobodies PDB: 6EY0 (porM-nanobody complex); PDB: 5DA0 (STAS-nanobody complex); PDB: SOVW (BtuF-nanobody complex); PDB: 5O2U (HIVp24-nanobody complex).

In addition to the two dsDNA repair templates, the disclosure comprises introducing into eukaryotic cells a clustered regularly interspaced short palindromic repeats (CRISPR)-Cas (CRISPR-associated proteins) system. The disclosure is illustrated using a Cas9 enzyme, but it is expected that other CRISPR systems and Cas enzymes can be used. In embodiments, any type II CRISPR system/Cas enzyme is used. In embodiments, the type II system/Cas enzyme is type II-B. In embodiments, that Cas enzyme comprises Cpfl. A sequence encoding the Cas enzyme may be used, or the Cas enzyme may be delivered to cells as a component of an RNP. The Cas enzyme may be a separate protein, or present in a fusion protein. In embodiments, the Cas enzyme is an engineered Cas9 and may exhibit, for example, a broad PAM range and/or high specificity and activity. Any protein described herein may include a nuclear localization signal.

In embodiments, the disclosure includes introducing two dsDNA repair templates, the Cas enzyme, optionally a trans-activating crRNA (tracrRNA), and a guide RNA. Suitable tracrRNAs are known in the art and can be adapted for use with the methods of this disclosure. In embodiments, a single RNA that combines components may be used in the form of a single guide RNA (sgRNA). In a non-limiting embodiment, the disclosure comprises use of three plasmids, wherein plasmid 1 encodes a sgRNA targeting genomic DNA as well as the Cas9 or other suitable Cas enzyme; plasmid 2 comprises the DNA template encoding the edit (mORF) and a first display epitope, and plasmid 3 comprises the DNA repair template encoding the edit (mORF) and the second display epitope.

The sgRNA may be provided as crRNA. The sgRNA is programmed to target specific sites so that the construct comprising the two dsDNA repair templates are integrated correctly, and thus target the chromosome locations, and the plasmid in the case where the dsDNA repair templates are provided on one or more plasmids. Methods for designing suitable guide RNAs, including sgRNAs, are known in the art such that guide RNAs having the proper sequences can be designed and used, when given the benefit of the present disclosure. The disclosure included introducing these RNA polynucleotides by way of coding in the dsDNA repair templates, or by introducing the RNA polynucleotides directly, and/or by including the RNA polynucleotides in an RNP. In embodiments, the two dsDNA repair templates comprise a secretion signal. In one non-limiting embodiment, an Ig heavy chain V-region precursor sequence can be used as the secretion signal. Additional and non-limiting embodiments include those that are functional in the pertinent cell type, such as mammalian cells, representative examples of which include signal sequence for interleukin-7 (IL-7) described in U.S. Pat. No. 4,965,195; the signal sequence for interleukin-2 receptor described in Cosman et al. ((1984), Nature 312:768); the interleukin-4 receptor signal peptide described in EP Patent No. 0 367 566; the type I interleukin-1 receptor signal sequence described in U.S. Pat. No. 4,968,607; the type II interleukin-1 receptor signal peptide described in EP Patent No. 0 460 846; the signal sequence of human IgG (which is METDTLLLWVLLLWVPGSTG (SEQ ID NO:9); and the signal sequence of human growth hormone (MATGSRTSLLLAFGLLCLPWLQEGSA (SEQ ID NO:10)). Many other signal sequences are known in the art and can be adapted for use in the compositions and methods of this disclosure. Certain non-limiting embodiments of the disclosure use a murine Ig kappa derived secretion signal that has the sequence METDTLLLWVLLLWVPGSTGD (SEQ ID NO:11). In some embodiments, the signal peptide may be the naturally occurring signal peptide for a protein of interest or it may be a heterologous signal peptide.

The type of eukaryotic cells that are modified, such as to comprise a homozygous insertion as described herein, are not particularly limited. In embodiments, the eukaryotic cells are mammalian cells. In embodiments, the cells are human cells. In embodiments, the cells are non-human animal cells, including but not limited to mammalian, fungal, insect, or algae or plant cells. In embodiments, the cells are canine, feline, murine, bovine, porcine, non-human primate, fish, or avian cells. In embodiments, compositions of this disclosure may be delivered a plant or to one or more plant cells, which may be present in intact plants, in a part of a plant that has been removed from a plant, or in a population of plant cells, such as cells grown in culture, or single plant cells. The term “plant cell” as used herein refers to protoplasts, gamete producing cells, and includes cells which regenerate into whole plants. Plant cells include but are not necessarily limited to cells obtained from or found in: seeds, suspension cultures, embryos, meristematic regions, callus tissue, leaves, roots, shoots, gametophytes, sporophytes, pollen, and microspores. Plant cells can also be understood to include modified cells, such as protoplasts, obtained from the aforementioned tissues. In embodiments, the disclosure provides plant products, which may be the plants themselves, or a product obtained directly from, or derived from, a plant subjected to the described method. In embodiments, the plant comprises a tree and the plant-derived commercial product is pulp, paper, a paper product, or lumber. In another embodiment, the plant is a grain and the plant-derived commercial product is bread, flour, cereal, oat meal, or rice. In another embodiment, the plant-derived commercial product is a biofuel or plant oil. In another embodiment, the plant-derived commercial product is a textile, such as a cotton-based textile. In embodiments, the plant is an ornamental plant. In embodiments, the plant is any type of cannabis. In embodiments, the plant is any variety of maize.

In embodiments, the eukaryotic cells are cancer cells, immune cells, or cells of a particular tissue, or organ. In embodiments, the cells comprise stem cells. In embodiments, the stem cells are induced stem cells, or are stem cells isolated from an individual. In embodiments, the stem cells are totipotent, pluripotent, or multipotent stem cells. In embodiments, the cells are hematopoietic stem cells. In embodiments, the stem cells are isolated or induced stem cells. In embodiments, the stem cells comprise embryonic stem cells. In embodiments, the disclosure comprises transgenic, non-human eukaryotic animals constructed using the described compositions and methods, which may be produced using, for example, isolated or induced stem cells.

In embodiments, the disclosure provides for removable or non-removable insertions. In embodiments, the disclosure provides for iterative editing by configuring the dsDNA repair templates to allow for removal of the epitopes from the chromosomes. Non-limiting examples of such configurations are illustrated, for example, by the figures. In embodiments, sequences encoding recombinase recognition sequences are included in the dsDNA repair templates. In embodiments, a pair of recombinase recognition sequences flank a segment of the dsDNA repair template that comprises or consists of a sequence encoding some or all of a secretion signal, a sequence encoding an epitope, a sequence encoding a transmembrane domain, and a sequence encoding a ribosome skipping sequence. In embodiments, the recombination recognition sequences flank at least the display epitope, or only the display epitope. Expression of a suitable recombinase in the nuclease of the cell will accordingly result in excision of such segments from the chromosomes. The type of recombinase and its recognition sequences are not particularly limited. In embodiments, the recombinase comprises Cre recombinase, and is used with loxP sites; a Flp Recombinase which functions in the Flp/FRT system; a Dre recombinase which functions in the Dre-rox system; a Vika recombinase which functions in the Vika/vox system; a Bxb 1 recombinase which functions with attP and attB sites; a long terminal repeat (LTR) site-specific recombinase (Tre), or other serine recombinases, such as phiC31 integrase which mediates recombination between two 34 base pair sequences termed attachment sites (att) sites. In embodiments, the spacer sequences between the inverted repeats of recombinase sites can be varied to ensure site-specific recombination only between homotypic variants flanking a gene but not between heterotypic variants that may flank another gene. These embodiments include the variants of the Cre-lox system that provide additional levels of specificity and prevent their cross-recombination. In embodiments, the removal of the epitopes can also be catalyzed by the site-specific excision using a second genome editing reaction involving either one or two single guide RNAs (sgRNA). In these embodiments a single cleavage can result in a frame shift to eliminate the epitope tag downstream of a skipping peptide or two cleavage events can excise the entire epitope cassette.

In embodiments, the recombinase can be provided by an extrachromosomal element, such as a plasmid. The presence of the extrachromosomal element may be transient. Further, expression of the recombinase may be inducible. In embodiments, expression of the recombinase may be controlled by a repressor. In embodiments, expression of the recombinase may be from an inducible promoter that is operably linked to the sequence encoding the recombinase. The DNA sequences of a wide variety of inducible promoters for use in eukaryotic cells are known in the art, as are the agents that are capable of inducing expression from the promoters. In embodiments, engineered regulated promoters such as the Tet promoter TRE which is regulated by tetracycline, anhydrotetracycline or doxycline, or the lad-regulated promoter ADHi, which is regulated by IPTG (isopropyl-thio-galactoside) may also be used. In embodiments, the activity or localization of the recombinase can be regulated. These embodiments include but are not limited to the use of tamoxifen-based relocalization of a recombinase to the nucleus or ligand-induced dimerization of the enzyme.

Induction of recombinase expression from an inducible promoter, dimerization, and relocalization of an existing recombinase are considered to be types of recombinase activation. In embodiments, the disclosure provides for use polynucleotides that encode a recombinase, such as the Flp recombinase, as well as a fluorescent protein, such as blue florescent protein, to facilitate selection expressing Flp recombinase (e.g., Flp-P2A-BFP) during sorting. Thus, the disclosure includes coupling the recombinase to any suitable selectable marker to select cells that express the recombinase

In embodiments, the disclosure comprises introducing into eukaryotic cells two dsDNA repair templates as described herein, each encoding a distinct epitope, allowing cell surface expression of the distinct epitopes, and separating cells that express both epitopes (thereby separating cells with a homozygous insertion) from cells that do not express both epitopes. Cells with homozygous expression of the two distinct epitopes may be separated using any suitable binding partners that can specifically bind the epitopes and are thus considered high affinity binders. In embodiments, separation of the cells may be performed immunologically using distinct antibodies or epitope binding fragments of antibodies, that separately recognize the epitopes with specificity. Suitable binding partners include but are not limited to antibodies, Fabs, scFvs, single domain antibodies (sdAbs, VHHs or nanobodies), affibodies or Darpins. Embodiments of the disclosure are shown using FACS separation. Thus, in embodiments, two distinct antibodies are used in methods of this disclosure, one of which binds with specificity to a first epitope and is labeled with a first detectable label, and a second antibody which binds with specificity to a second epitope, and is labeled with a second detectable label that produced a signal that is distinct from the first label. Such approaches provide for, as discussed and demonstrated further below, identification and separation of cells comprising homozygous insertions. The type of label is not particularly limited, and many suitable labels are commercially available, and can be conjugated to antibodies using known techniques. In embodiments, the label produces a detectable signal that is outside the visible range, thereby limiting interference in a case where, for example, a fluorescent protein may be used as the tag. However, other configurations are encompassed this disclosure. For example, the first and second epitope can comprise any fluorescent proteins, provided their excitation and emission spectra are separable. These include but are not limited to GFP, mCherry, mTAGBFP2, mPlum, YFP, mPapaya, mStrawberry, BFP, Sirius, and the like. In embodiments, the detectable labels produce a signal that comprises UV light (<380 nm), visible light (380-740 nm) or far red (>740 nm). In embodiments, one or more dyes can be used, such as for FACS sorting. Any suitable dyes and combinations of dyes may be used, such dyes being recognized by those skilled in the art.

When given the benefit of the present disclosure, those skilled in the art will understand how to control the pertinent FACS windows to achieve efficient separation. In embodiments, the disclosure provides for concurrent separation of cells that express both epitopes, while activating the recombinase, to provide a homogenous population of cells comprising a homozygous insertion, but from which the epitopes have been removed. In embodiments, removal of the epitopes is scarless, with the potential exception of residual nucleotides from the recombinase-mediated excision of the epitope coding sequences.

Control over excision can be provided by configuring the location of the cassette comprising the secretion signal, the sequence encoding the epitope, the sequence encoding the transmembrane domain, and the sequence encoding the ribosome skipping sequence. For example, this cassette can be positioned either N- or C-terminal to a homology arm that comprises the tag. Activation of the recombinase can be performed, for example, within one hour before or after FACS sorting.

In embodiments, cells that are modified and isolated according to this disclosure, and from which the epitopes may have been removed, are subjected to at least a second round of modification, which can be performed for the same or different alleles, and with the same or different tags and epitopes. In embodiments, loxP and/or its variants can be used to limit or prevent recombination between non-homologous alleles.

In embodiments, the disclosure comprises providing a treatment to an individual in need thereof by introducing a therapeutically effective amount of modified eukaryotic cells as described herein to the individual, such that the homozygous insertion treats, alleviates, inhibits, or prevents the formation of one or more conditions, diseases, or disorders. In embodiments, the cells are first obtained from the individual, modified according to this disclosure, and transplanted back into the individual. In embodiments, allogenic cells can be used. In embodiments, the modified eukaryotic cells can be provided in a pharmaceutical formulation, and such formulations are included in the disclosure. A pharmaceutical formulation can be prepared by mixing the modified eukaryotic cells with any suitable pharmaceutical additive, buffer, and the like. Examples of pharmaceutically acceptable carriers, excipients and stabilizers can be found, for example, in Remington: The Science and Practice of Pharmacy (2005) 21st Edition, Philadelphia, Pa. Lippincott Williams & Wilkins, the disclosure of which is incorporated herein by reference.

In embodiments, the disclosure comprises a kit for use in making modified eukaryotic cells such that they comprise a homozygous insertion. In embodiments, the kit comprises one or more cloning vectors, the vectors comprising the elements discussed above for producing the dsDNA repair templates. The dsDNA repair templates may be provided with suitable cloning sequences such that the user can select and introduce desired 5′ and 3′ homology segments, or these segments may be included. The vector(s) may include sequences encoding the epitopes, or cloning sequences for introducing sequences encoding the epitopes. sgRNAs and/or a Cas enzyme may also be provided with the kit. The kit may also include detectably labeled high affinity binding partners. In embodiments, the kit comprises two plasmids that include different multi-cloning sites for inserting a mORF and different surface display epitopes, such that a different surface display epitopes are expressed by each plasmid. The plasmid may include, for example, a TMD coding sequence. The plasmids may also comprise different surface display epitopes so that the user need only clone in the mORF into each plasmid.

The following examples and the corresponding figures are intended to illustrate, but not limit the disclosure:

### EXAMPLE 1

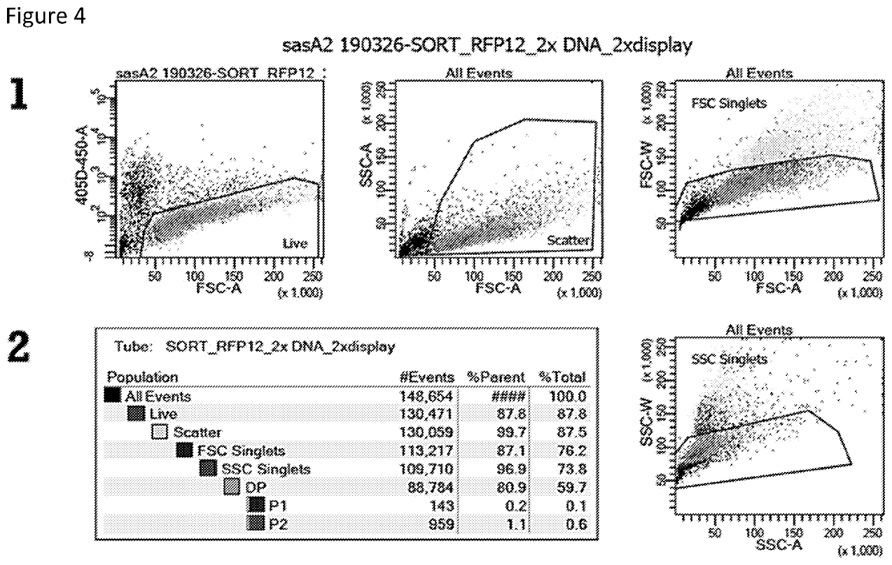

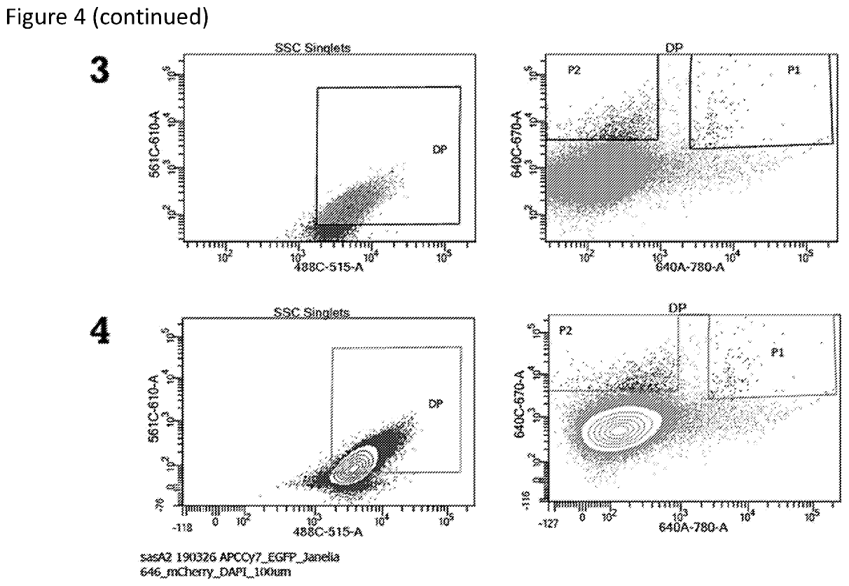

This example provides materials and methods used in various embodiments of this disclosure, and a non-limiting demonstration of using mCherry as a tag with STAS and porM as surface epitopes, as shown in FIG. 4.

Transfection of sgRNA and Repair Template plasmids. To initiate transfection, suitable cells, typically 293F cells, are first counted using a hemocytometer. A suitable number of cells, typically 0.1-0.4×10⁶ cells/ml, are plated into single wells of a 24 well tissue culture treated plate. Final volume of cells is 1 ml/well. Cells are grown in GIBCO Freestyle 293 medium supplemented with 2% FBS in an incubator at 37° C., 8% CO₂ at appropriate humidity of approximately 80%. Cells are grown to between 70-90% confluency before transfection, generally within one to two days. Cells are washed with warm medium without FBS and resuspended in 0.5 ml warm medium/well.

Preparation of DNA for transfection. Representative protocol using, for each well, two suitable tubes, referred to for convenience as Tube A and Tube B. Tube A: 2 μl Lipofectamine 2000+25 μl warm Opti-MEM medium. Tube B: Plasmid DNA (sgRNA+Cas9+Repair templates)+25 μl warm Opti-MEM medium. Plasmids are used in equimolar concentrations. The total amount of DNA in Tube B can be 500 ng (1×) or 1000 ng (2'). For CRISPR experiments involving the display epitope, three plasmids were transfected. Plasmid 1: Encodes the sgRNA targeting genomic DNA as well as the Cas9 enzyme. Plasmid 2: Repair template encoding the edit+display epitope 1. Plasmid 3: Repair template encoding the edit+display epitope 2. For use in multi-well transfections, master mixes of Tube A and Tube B are used. The contents of Tube A and Tube B are mixed and incubated at room temperature for 10-15 mins and aliquoted evenly over the cells, with gentle shaking after the addition. Cells are incubated for a suitable period, such as 12 hours, after which viability is determined. The cells are aspirated with medium and washed 1× with 1 ml/well Gibco Freestyle 293 medium, supplemented with 2% FBS and resuspended in 1 ml of this medium. Expansion of cells is monitored for three to 4 days post-transfection and the cells passaged from the 24 well plate to a 6-well plate. Cells typically reach 100% confluency in the 6 well plate 7 days post transfection, after which they are ready for FACS sorting. Larger cell populations can be used in the same manner, except the cells are moved to a 10 ml suspension culture after 7 days. Cells can take a further 6-8 days to adapt to the suspension culture. Once adapted cells can be expanded to larger suspension volumes, if required. Cells are passaged every 3-5 days. Cells can be kept in suspension for up to 120 days prior to FACS sorting.

When cells are moved from adherent plates to a suspension culture, the media is supplemented with 2% FBS. The FBS can be removed after the first cell passage. After moving cells to suspension, white flakes in the media may be observed after 4-5 days. These can be removed by first transferring the culture to a falcon and letting the flakes settle at the bottom. The cell suspension is then transferred to a new flask to remove the flakes. If suspension cells stop growing or show low viability, they are spun at 100×G, 5 min, 23° C. to pellet the cells. The supernatant is discarded, and the cells are resuspended in fresh, warm Gibco Freestyle 293 medium supplemented with 2% FBS. Thus, the timeline for expanding cells post transfection includes 1-4 days in 24 well plates, expansion in 6 well plates for three days, and expansion in 10 ml suspension culture for approximately 7 days, or longer.

FACS Sorting of Single Cell Clones using Two Display Epitopes.

A HEK 293F cell line was used in which both copies of the BYSL gene were pre-edited with a C-terminal GFP tag. Into this cell line repair templates were transfected to tag the gene copies of RRP12 with mCherry and the display epitopes (containing either STAS or porM as the display epitopes). The sequences of these and other constructs used to produce the results of this disclosure are provided below. Both BYSL and RRP12 are ribosome biogenesis factors. Cells were transfected with either 1× (500 ng) or 2× DNA (1000 ng) of DNA for the experiment. Editing of DNA using the display epitopes in wildtype 293F cells or any other cell type follows the same protocol as described here. The following color controls were used.

| No. | Color | Control cell line |
|---|---|---|
| 1. | GFP | 293F_BYSL_GFP or |
| | | 293F_WDR74_GFP |
| 2. | mCherry | 293F_wildtype transfected |
| | | with plasmid number |
| | | M022 (Utp24_tev_mCherry) |
| | | or 293F_Pes1_mCherry cell line |
| 3. | Dye: Janelia_646 | 293F_wildtype transfected with |
| | | plasmid M064 (expresses STAS |
| | | at cell surface), followed by |
| | | immunostaining with an |
| | | anti-STAS nanobody |
| | | labeled with Janelia-646 dye. |
| 4. | Dye: APC_CY7 | 293F_wildtype transfected with |
| | | plasmid M063 (expresses porM |
| | | at cell surface), followed by |
| | | immunostaining with an |
| | | anti-porM nanobody |
| | | labeled with APC_CY7. |
| 6. | Dead cell exclusion | Added to cells at a final |
| | dye (DAPI) | concentration of 100 ng/ml. |
| 7. | Background/ | 293F wildtype cells |
| | Negative control | |

To determine the optimal DAPI concentration, a titration series was first performed wherein increasing concentrations of DAPI were mixed with 293F wildtype cells followed by FACS analysis.

FACS sorting of single cell clones: Cell sample preparation is carried out on the same day as the FACS sort. Immunostaining: Immunostaining is used to select cells with both STAS and porM display epitopes using fluorescently labeled nanobodies against both proteins. For this anti-STAS_Janelia646 and anti-porM_ APC-Cy7 labelled nanobodies were used, but the dyes can be switched to use anti-STAS_APC-Cy7 and anti-porM_Janelia646, or any other suitable markers. Preparation of cell samples: Cells are spun down at 100×G, 5 min, 4° C. Supernatant is discarded and cells washed 1× with 1× PBS, 0.1% BSA at 100×G, 5 min, 4° C. Cells were resuspended in 1× PBS, 0.1% BSA so that the final concentration was between 1-10×10⁶ cells/ml. (Cell samples and cell color controls that do not require immunostaining are also prepared). FACS sorting. For surface immunostaining, labeled nanobody is added to between 100-200 μl of cell suspension. For nanobodies labeled with at least 1 dye/protein the final nanobody concentration is at least 10 nM. For suboptimal dye-protein labeling, the concentration of added nanobody can be increased. For example, if labeling efficiency is 1 dye/25 protein molecules, nanobody concentration can be increased to 10× to 250 nM. Cells are incubated on ice in dark for 15 mins. After harvesting wash cells 2× with 1× PBS, 0.1% BSA to remove free dye. The volume per wash is 1 ml. After washing, labeled cells are resuspended (1× PBS, 0.1% BSA) in a small volume (100-200 μl ). This sample is FACS sorted. Immunostaining of color controls is carried out in the same manner. Sorting of single cell clones. Single cell clones were sorted into 96 well plates pre-aliquoted with warm GIBCO Freestyle 293 medium supplemented with 2% FBS. A total of 140 μl of medium was aliquoted into each well. Each plate received a total of 60 single cell clones from the FACS sorter. Post sorting the plates were immediately transferred to an incubator at 37° C., 8% CO² and adequate humidity. For the results shown in FIG. 4, tagging of RRP12 with mCherry, cells were sorted for both 2× DNA (and 1× DNA as shown in the table below) transfected cells. 120 clones (Two 96 well plates) were sorted for each sample.

Post sorting for insertion verification. For the two samples sorted in FIG. 4, the survival rates were as follows.

| Sample | Clones sorted | Clones survived |
|---|---|---|
| 2X DNA | 120 | 45 |
| 1X DNA | 120 | 44 |

Healthy clones usually reach 100% confluency in 96 well plates after 2 weeks post-sort. These cells are washed gently with 140 μl of medium and each clone is transferred to a separate 24 well plate, supplemented with 1 ml of GIBCO Freestyle 293 medium supplemented with 2% FBS. Genomic DNA extraction: Once clones have reached 100% confluency in 24 wells, genomic DNA extraction is performed for the purpose of PCR validation of the edits approximately 3-4 days after moving cells to 24 well plates. PCR verification is performed using standard approaches. Generally, cells are washed with 1 ml of medium and resuspended in 200 μl of GIBCO Freestyle 293 medium supplemented with 2% FBS. 20 μl of cells are placed into 500 μl of QuickExtract DNA Extraction Solution (Lucigen), on ice. The mixture is vortexed for 15 seconds, transferred to 65° C., incubated for 6 minutes, vortexed for 15 seconds, transferred to 98° C. and incubated for 2 minutes. DNA is stored at −20° C. temporarily, or at −80° C. for longer term storage. 30 μl of solution as extracted DNA template is used in a 50 μl PCR reaction.

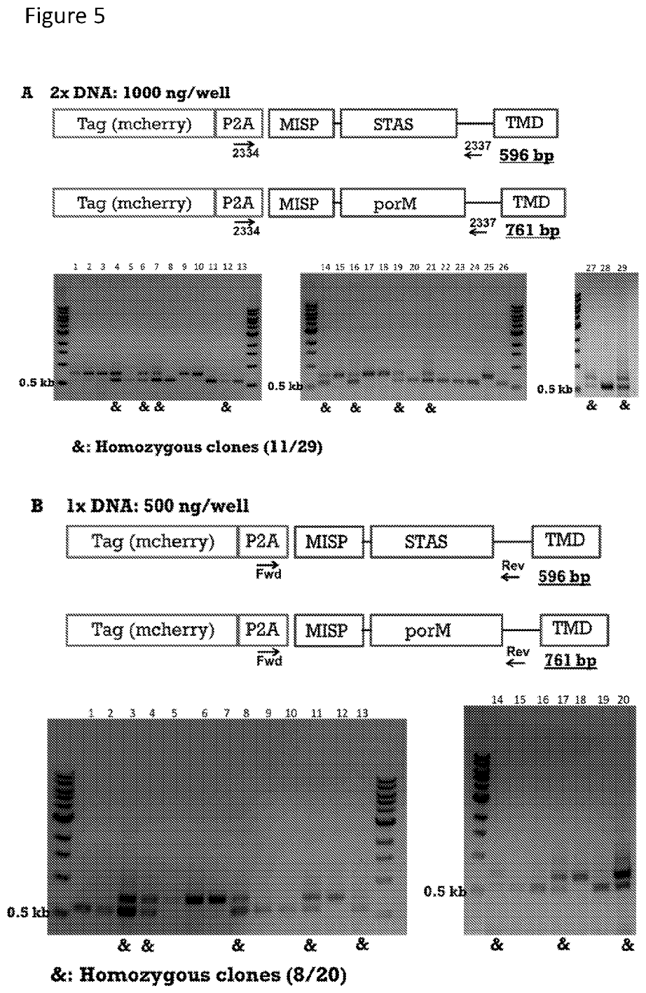

PCR validation to identify homozygotes. As shown in FIG. 5, PCR validation was first carried out to select homozygously edited clones based on double amplification of both STAS and porM coding DNA in the same PCR reaction. This analysis was carried out for both the 1× and 2× DNA experiments (FIG. 5). The PCR reaction components were as follows:

| No. | Component | Amount (μl) |
|---|---|---|
| 1. | H₂O | 8 |
| 2. | 5× Phusion HF buffer | 10 |
| 3. | dNTP | 1 |
| 4. | Genomic DNA | 30 |
| 5. | Fwd primer (2334) | 0.25 |
| 6. | Rev primer (2337) | 0.25 |
| 7. | Phusion DNA Polymerase | 1 |

PCR program: lower_annealing_1kb_per on 3prime

FIG. 5 shows PCR validation of homozygously edited single cell clones. For the 2× DNA experiment (FIG. 5, panel A) 11/29 clones were positive for both STAS and porM DNA (homozygotes). For the 1× DNA (FIG. 5, panel B) experiment 8/20 clones are positive for both STAS and porM DNA (homozygotes).

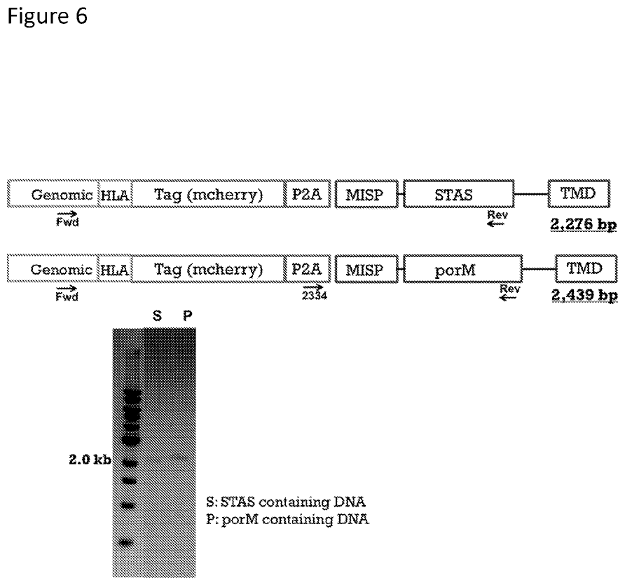

PCR validation to verify complete and site-specific integration of insert DNA: As shown in FIG. 6, a single homozygote clone was selected to verify complete and site-specific genomic integration of the insert. PCR primers were designed to specifically amplify the entire region of insert DNA extending from upstream of the left homology arm right up to the display epitope (STAS/porM) “HLA” means homology left arm. MISP stands for murine immunoglobin signal peptide.

The day after PCR validation the PCR validated clone was moved to a single well in a 6-well plate. The total volume of the medium was 3 ml Gibco Freestyle 293 medium supplemented with 2% FBS. Cells are passaged and after 3-4 day expanded into two wells of a 6-well plate. Once cells reach 100% confluency, they are moved to a 10 ml suspension culture grown in Gibco Freestyle 293 medium supplemented with 2% FBS. Clones can be preserved as follows. After 2-3 passages in suspension, cells are split into a 50 ml culture prior to banking.

Protocol for banking of clones. Cells are spun down at 100×g, 4° C., 4 min, the supernatant is discarded. The cell pellet is resuspended in cold banking medium (90% Gibco freestyle 293 medium+10% DMSO) so that the final concentration of cells is between 5-8×10⁶ cells/ml. Cells are aliquotted as 1 ml aliquots into labeled vials and transferred to a cooling container filled with 250 ml of 100% isopropanol and stored at −80° C. overnight. Cooled vials are transferred to liquid nitrogen the next day. Cells can be thawed and used according to standard techniques.

### EXAMPLE 2

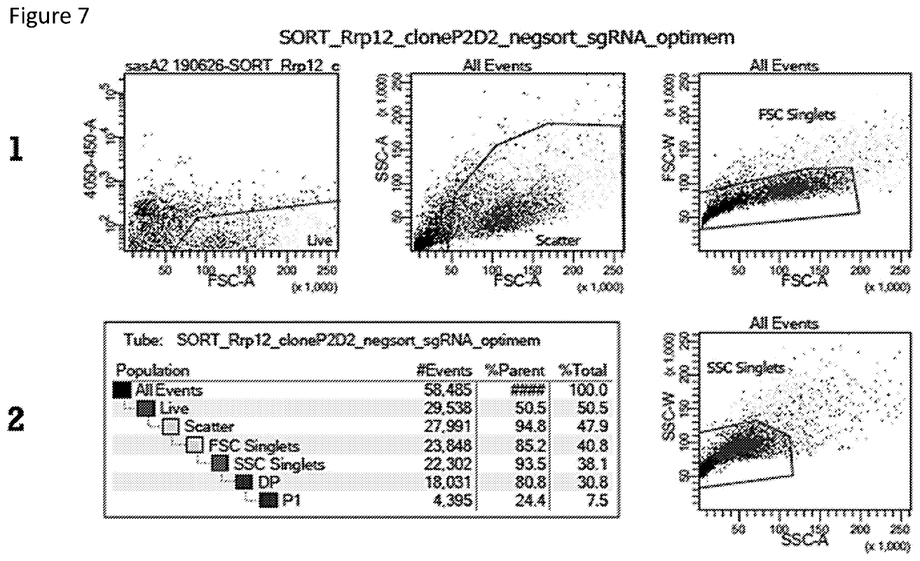

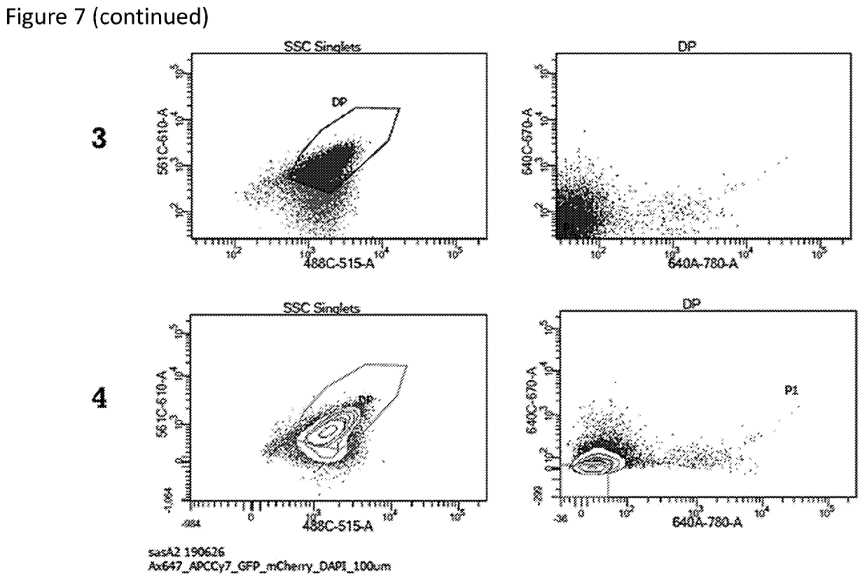

This Example provides non-limiting protocols and additional homozygous editing, homozygously edited clone production and isolation, and PCR validation, as shown in FIGS. 7-10.

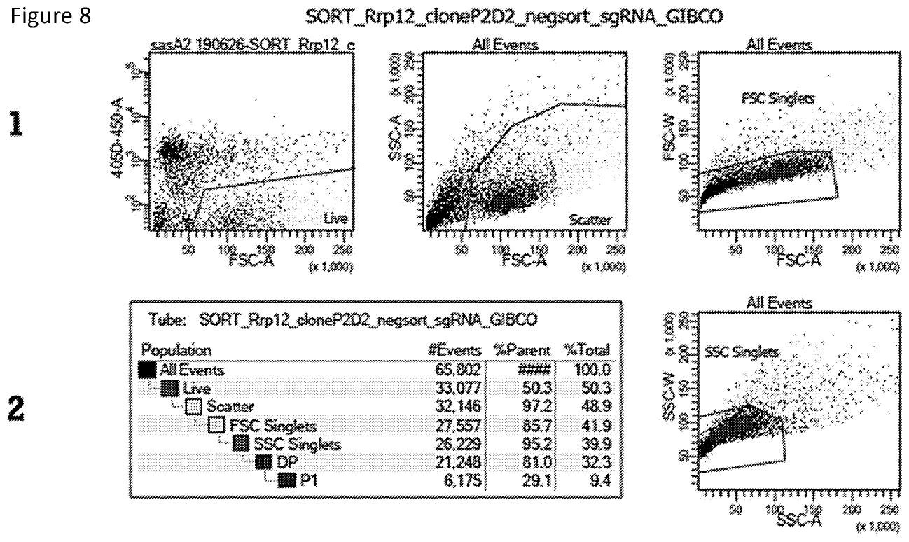

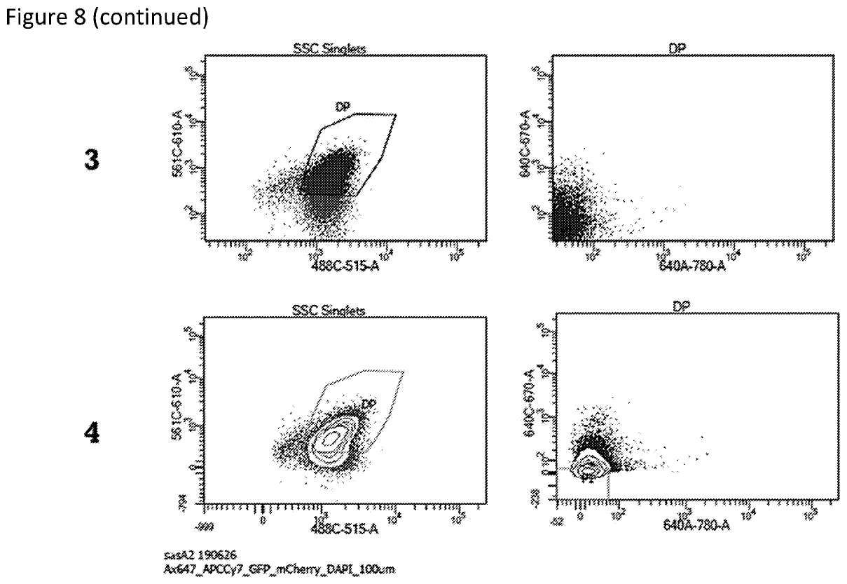

On Day 1, RRP12_mCherry clone_P2D2 (positive for STAS/porM display) cells are plated in an entire 24 well plate and grow overnight. Cell count for plating is 0.13×10⁶ cells/ml. On Day 2, begin transfection once cells have reached between 70-90% confluency. For transfection, Tube A contains a master mix of 50 μl Lipofectamine 2000+625 μl optimum and Tube B contains 12.5 μg sgRNA M084 (500 ng/well)+625 μl optimum. The contents of tube A and tube B are mixed and incubated at room temperature for 10-15 mins. 52.7 μl is transfected into each well and the transfected cells are left overnight. Results in FIG. 7 were obtained using Opti-MEM medium. The results in FIG. 8 were obtained using GIBCO Freestyle medium instead of Opti-MEM. The rationale is that since cells do not grow well in Optimum a transfection in Gibco will help cells recover quickly. Gibco freestyle medium is FBS free during transfection. On Day 3 the cells are washed with 1 ml/well Gibco Freestyle medium, supplemented with 2% FBS, then resuspended cells in 1 ml of the medium. Cells are allowed to recover for approximately one day. On Day 5 when the cells are growing and approaching 100% confluency the cell culture is expanded by transferring to a single 6-well plate. The cells reach about 100% confluence before initiating the FACS sorting. On Day 7 FACS sorting is performed using a standard approach for sample preparation. In this example, the samples comprise RRP12_mCherry-BYSL_GFP_STASJanelia646_porM_ApcCy7. Two samples are sorted, as follows.

| No | Sample |
|---|---|
| 1. | RRP12_mCherry-BYSL_GFP_STASJanelia646_porM_ApcCy7 Sample transfected with sgRNA targeting murine immunoglobulin signal peptide. Plasmid transfection was performed in Opti-MEM medium |
| 2. | RRP12_mCherry-BYSL_GFP_STASJanelia646_porM_ApcCy7 Sample transfected with sgRNA targeting murine immunoglobulin signal peptide. Plasmid transfection was performed in Gibco Freestyle medium |

The following samples were used at color controls

| No. | Sample | Color |
|---|---|---|
| 1. | 293f_WDR74 | GFP |
| 2. | 293f_Pes1 | mCherry |
| 3. | 293f + plasmid M063 + anti-porM_ApcCy7 (immunostain) | ApcCy7 |
| 4. | 293f + plasmid M064 + anti-STAS_Janelia646 (immunostain) | Janelia646 |
| 5. | 293F_wildtype | No color |

DAPI is used as the dead cell exclusion dye at a concentration of 100 ng/ml. Results from Opti-MEM transfection are shown in FIG. 7. Single cell clones were collected from window P1. Results from GIBCO Freestyle transfection are shown in FIG. 8. Single clones were collected from window P1.

Collection of single cell clones. A single 96 well plate was collected for each sample. The samples were collected using the index sorting program which allows the user to match each collected clone with its corresponding position in the gate used for the sort. Index sorting collects 96 clones/plate, unlike regular sorts, which collect 60 clones/plate. Also, index sorting does not allow for a pool of cells to be collected in a single well at the corner of the plate. Regular sorts use this as a way to monitor cell growth as well as to find the right plane in which to focus the plate under the microscope. We also used conditioned media to grow the sorted cells.

Preparation of conditioned media: 293f cells were grown in 25 ml suspension for 2 days. GIBO serum free medium supplemented with 1× Anti-Anti was used.

After 2 days cells were spun down at 100×G, 5 min, and the supernatant was filtered through a 0.2 μm filter using a syringe. Fresh GIBCO serum free medium was then added to the filtered medium in the ratio 1:1. FBS was added to a final concentration of 2%.

At Day 21 (2 weeks post sort), plates were imaged and wells with clear clumps of growing cells were marked. The results were as follows:

| | Sorted into | Wells showing |
|---|---|---|
| Sample | (+2% FBS) | cell growth |
| Opti-MEM transfect (Plate 1) | Fresh Gibco | 10/48 |
| Opti-MEM transfect (Plate 1) | Conditioned Gibco | 13/48 |
| Gibco transfect (Plate 2) | Fresh Gibco | 16/48 |
| Gibco transfect (Plate 2) | Conditioned Gibco | 19/48 |

Conditioned media shows slightly higher number of wells with growth. 24 clones with the largest growing cell clumps were transferred to single wells of a 24 well plate. Each well contained 1 ml of Gibco freestyle medium+2% FBS. At Day 26 8 clones were selected for PCR based validation, as follows:

| Clone | Sample type | Sorted into (+2% FBS) |
|---|---|---|
| P1C6 | Opti-MEM transfect | Fresh Gibco |
| P1C11 | Opti-MEMtransfect | Fresh Gibco |
| P1E6 | Opti-MEMtransfect | Conditioned Gibco |
| P1F8 | Opti-MEMtransfect | Conditioned Gibco |
| P2C5 | Gibco transfect | Fresh Gibco |
| P2C12 | Gibco transfect | Fresh Gibco |
| P2E2 | Gibco transfect | Conditioned Gibco |
| P2E6 | Gibco transfect | Conditioned Gibco |

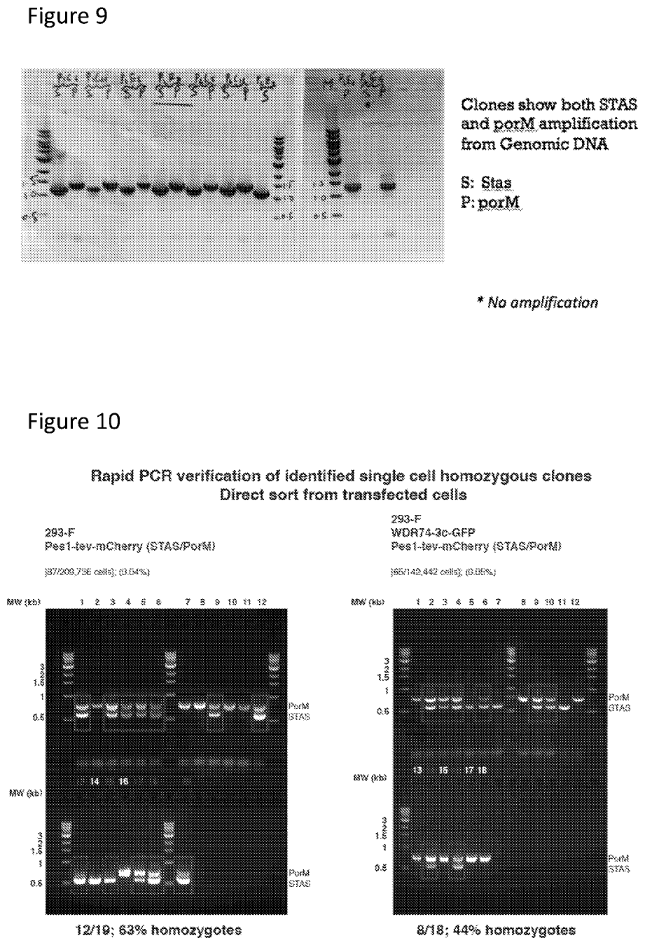

PCR validation: Since each sample contains porM and STAS domains, two PCR amplifications are be carried out on each sample. Results are shown in FIG. 9. Genomic DNA amplification was carried out as per the standard protocol using the Lucigen QuickExtract solution.

Sequencing: Both PCR products from 4 clones were column purified and sequenced using primer 2665 (mCherry_seq_fwd).

| | | | Display |
|---|---|---|---|
| No | Clone | Sequencing result | inactivation |
| | P1C6_STAS | Single nucleotide insertion (G) resulting in premature STOP codon | Yes |
| 2. | P1F8_porM | Sequence unchanged | No |
| | P1F8_STAS | Sequence unchanged | No |
| 3. | P2C12_porM | 62 base pair sequence inserted; premature stop codon | Yes |
| | P2C12_STAS | 75 base pair sequence inserted; premature stop codon | Yes |
| 4. | P2E2_porM | Sequence unchanged | No |
| | P2E2_STAS | Sequence unchanged | No |

Sequence Alignment of Inserts

NCBI BLAST revealed that the insertions in clone P2C12 showed very high sequence identity with regions in the human genome

| | |
|---|---|
| porM insert (62 bp) | 98.6% sequence identity with a region in chromosome 15 |
| STAS insert (75 bp) | 100% sequence identity with a region in chromosome 18 |

Both reactions for clone_P2C12 show the inactivation of the STAS and porM display. This clone was transfected in Gibco freestyle medium and the grown in fresh Gibco freestyle medium+2% FBS. FIG. 9 shows representative PCR reactions used to verify inserts and for sequencing. Annotated sequences used in examples of this disclosure are provided below.

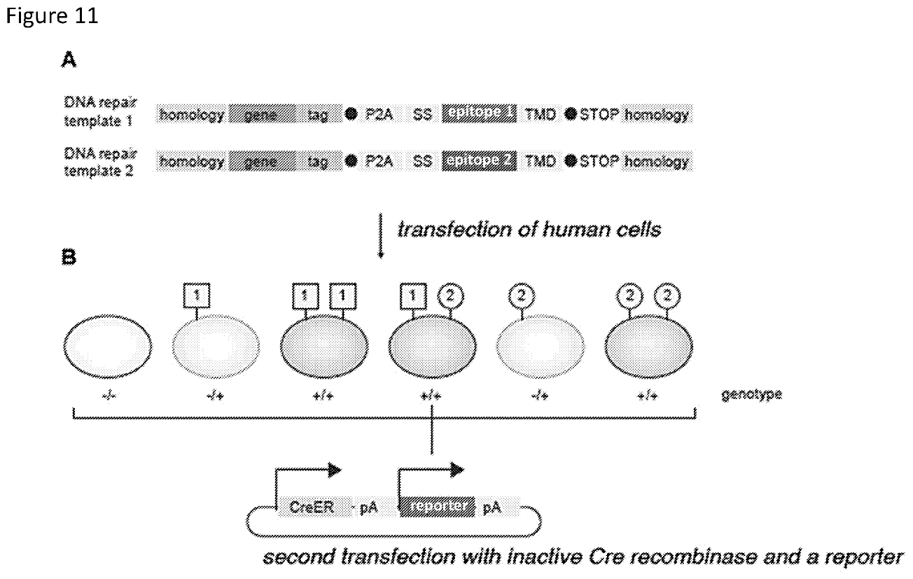

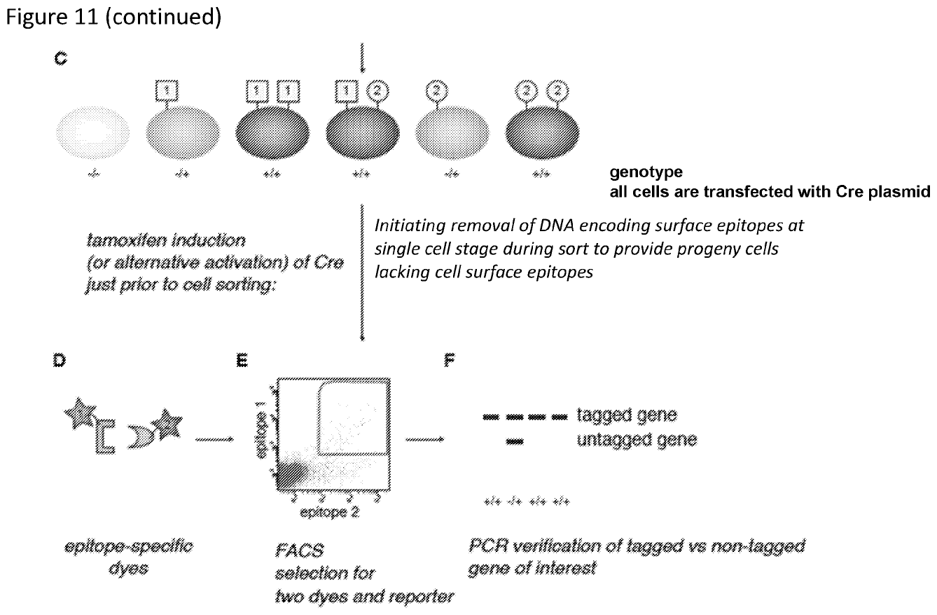

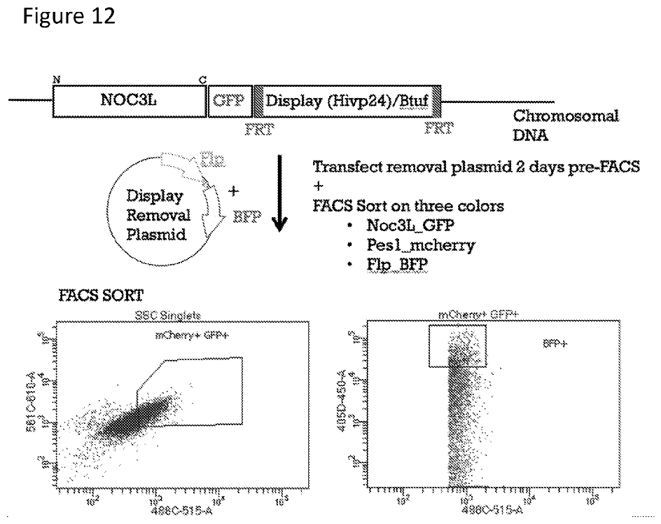

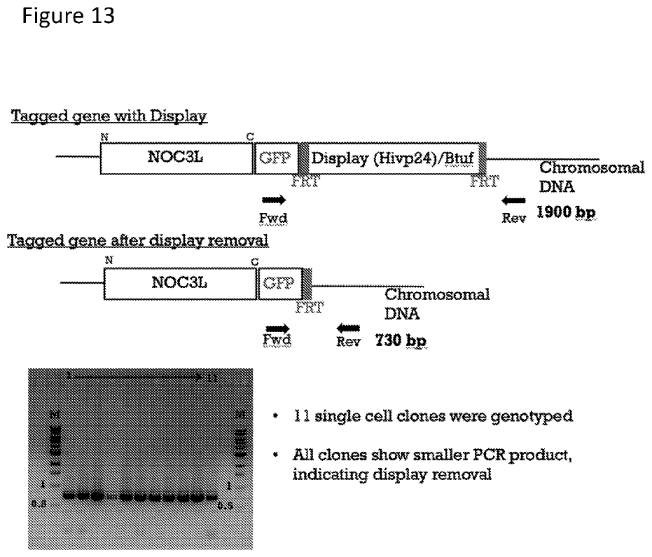

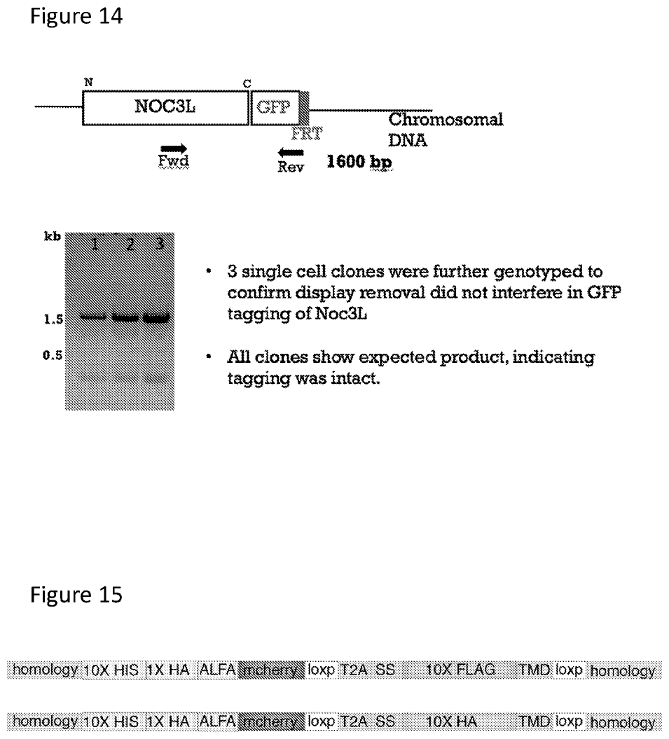

FIG. 11 provides a schematic demonstrating workflow for recombinase-mediated removal of cell surface epitopes, and relates to FIGS. 12-14, which show non-limiting examples of epitope recycling that can be used with, for example, FLP recombinase. This is performed by transfecting a plasmid that expresses the FLP recombinase into a cell line in which the Noc3L gene has been homozygously tagged using SNEAK PEEC using the compositions and methods described above. FLP recombinase excises the two display epitope sequences by targeting flanking, unidirectionally placed FRT recombinase target sites. Downstream of the FLP recombinase sequence is a 2a ribosome skipping sequence followed by the sequence of the blue fluorescence protein (BFP). FACS sorting was used to select single cell clones expressing Noc3L-GFP, mCherry and BFP. Single cell clones were grown for 2-3 weeks and genotyped using PCR to confirm removal of the entire display epitope from both Noc3L gene copies. We obtained 100% recycling of the Hiv p24 and Btuf display epitope sequences for all the clones screened. Additionally, screening of these clones showed that display epitope removal does not disrupt the editing of the cell lines, meaning the cells are still biallelically tagged Noc31-gfp, but without the display epitope. The mCherry signal was obtained from homozygous tagging of another gene in the same cell line, namely Pes1. The SNEAK PEEC display sequences for tagging Pes1 do not contain FRT recombinase sites and are thus not targeted by the FLP recombinase. Transfection and FACS sorting of single cell clones is shown in FIG. 12. FIGS. 13 and 14 show obtaining single cell clones and genotyping, confirming display removal (FIG. 13) and that display removal did not interfere with GFP tagging (FIG. 14).

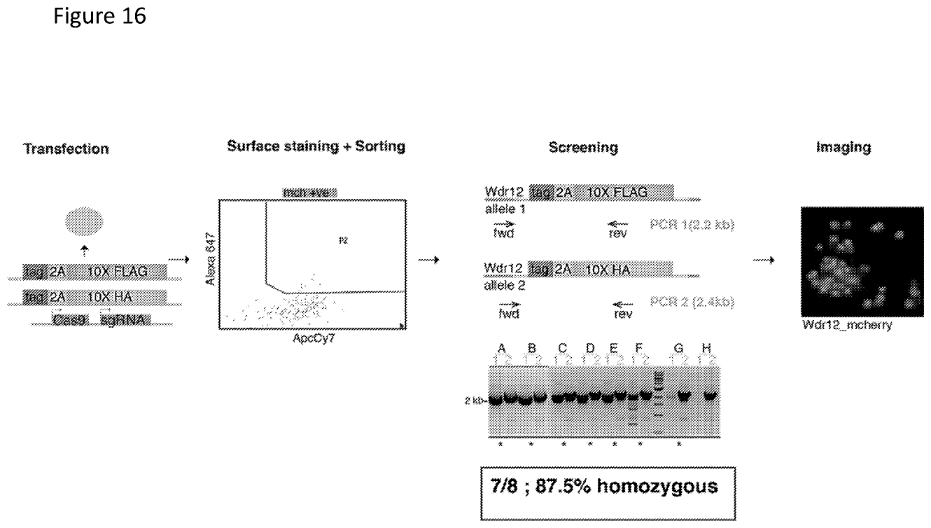

SNEAK PEEC was also performed using peptide epitope arrays as display epitopes, along with a ribosomal skipping sequence. The human ribosome biogenesis factor WDR12 was chosen for editing. The two DNA repair templates targeting WDR12 are as shown in FIG. 15. In FIG. 15, each repair template contains a homology arm, followed by a downstream multifunctional tag (10× His, 1× HA, ALFA, mCherry). This is followed by a downstream loxp site, T2A viral ribosome skipping sequence, secretion signal (SS), a peptide array of 10× HA for one repair template and 10× FLAG for the second repair template. This is followed by a transmembrane domain (TMD), loxp site and a homology arm. FIG. 16 shows homozygous editing of 7/8 (87.5%) of sorted cells. In FIG. 16, HEK293F cells were transfected with two repair templates targeting the C-terminus of the Wdr12 gene (as in FIG. 15), along with a plasmid expressing the Cas9 protein and an sgRNA targeting the last exon of Wdr12. Two flanking homology arms (600 bp each) in the repair templates match the genomic region either direction of the DNA cut site. Each repair template encodes a multifunctional fluorescent tag (10× His-HA-ALFA-mCherry) followed by a surface display containing either 10× FLAG or 10× HA arrays as a peptide epitope. Post transfection the cells were surface stained with commercially available anti-FLAG and HA antibodies conjugated with fluorophores Alexa 647 and Apc-cy7, respectively (Panel: Surface staining+Sorting). FACS sorting was used to select mCherry expressing cells that were also positive for Alexa647 and Apc-cy7 (Window P2). Single cell clones were collected and grown for two weeks prior to screening. Genomic DNA from eight of the fastest growing clones was subjected to two PCRs, each designed to detect correct knock-in of one of the repair templates (Panel: Screening). Of the first eight clones screened, seven (87.5%) were positive for both PCR products, indicating homozygous editing. Clones were then imaged to verify correct localization of tagged Wdr12 in the nucleolus. Images showed nucleolar accumulation of mCherry, signifying tagged Wdr12 is functional.

Annotated sequences with grids explaining annotations are as follows:

### Sequences of Repair Templates

1. RRP12_mCherry_SurfaceDisplay(porM)
(SEQ ID NO: 12)
¹CCGGCGAGGTTCCCAGGTGGGAC²⁴CCCAGGATGGTCTTGATCCCCTGACCTTGTGATCTGCCCACC
TCGGCCTCCCAAAGTGCTGGGATTACAGGCATGAGCCACCACGCCCAGCCATAGTCATCATTTTTA
ATAGCTTTGTATAATTTGCTTTTCTAATCCCTTTATTGGTAGGAAATTAGAGTTGTTTCCGACTTTG
GCCCTTAAATTGGGTTATGTGTAGGACTGCTTTGGAAACTAATGTTACTAGGGAAATGGTGTTGTA
AAGTTCTAGCTTCTGCGGGTTGTAAGTTACCTTTCAATGGAGGGATGGGTGGGCAGAGGGAGCTTT
GACCTTCTCTGGACATACATTAGAGGAAAAATGGAAGGGAGGCCTGTTTCCAGGGGGATAATTGT
GCCAAAGTGGAATGTCCAGGTCAGGACATGAGCCGTGTGGAAGCTGGAACCACGTGAGGTCTGCC
TAGTTCATGTGCTGGCCACCACCTGGAGGCCCCCTTCTCATCCCTGCTGGCGCTGGGGGTGAGCCA
TCATTTGGCAACAGGAGGGGGCCTCCTATTCTCAGCCAGATGTGACCCTTCCGTTCCTTGGCCCTG
CAGGAAGAAGATGAAGCTGCAGGGACAGTTCAAAGGCCTGGTGAAGGCTGCtCGGCGAGGTTCCC
AGGTGGGACACAAAAATCGCCGGAAAGATAGAAGACCC⁶⁹⁶gcggccgcc⁷⁰⁵GGGGGCACGGGAAGTGG
TGGATCAGCCGGTGGCACTGGTGGCTCTGCCGGAGGGTCAGCGGGAGCAGGGGGAGCCACAGGC
GGATCTACGGCTGGAGGGGCGACAACGGCCTCT⁸¹⁹gcgatcgctGGCGAAAATCTGTATTTTCAGGGAG
GAgCTAGCGGAAGCGGA⁸⁷⁰ATGGTCAGTAAGGGTGAGGAGGACAACATGGCTATAATCAAAGAGT
TTATGCGGTTTAAGGTCCATATGGAAGGTTCAGTTAATGGACATGAGTTCGAGATAGAAGGTGAG
GGTGAGGGGCGACCGTACGAAGGCACACAAACCGCAAAGTTGAAAGTCACCAAAGGTGGACCCT
TGCCCTTTGCTTGGGATATTCTCTCCCCTCAATTCATGTACGGCAGTAAGGCATACGTCAAACATCC
CGCTGACATCCCCGACTATCTGAAGCTGTCTTTCCCTGAGGGTTTTAAATGGGAGCGAGTGATGAA
CTTCGAGGACGGGGGAGTGGTAACAGTGACTCAAGATTCCTCTTTGCAGGACGGGGAGTTCATAT
ATAAAGTGAAACTGCGGGGTACGAACTTTCCAAGTGACGGtCCCGTAATGCAGAAGAAGACGATG
GGATGGGAGGCAAGCAGCGAGCGAATGTATCCTGAGGATGGAGCCCTTAAGGGAGAAATTAAGC
AACGGCTGAAGTTGAAAGATGGTGGACATTATGATGCTGAGGTTAAAACAACTTATAAAGCCAAG
AAACCAGTTCAGTTGCCAGGGGCGTATAACGTCAACATTAAACTGGACATTACATCTCACAATGA
AGATTACACAATCGTTGAGCAATATGAaCGCGCGGAGGGTCGGCACTCAACGGGTGGCATGGACG
AGTTGTATAAA¹⁵⁷⁸GGcgcgcccggaagcgga¹⁵⁹⁶gctactaacttcagcctgctgaagcaggctggagacgtggaggagaaccctggacct¹⁶⁵³
atgggctggtcatgtatcattctgtttctggtcgcaaccgcaactggagtgcattcacaggtgcagctcggcggaccgACGAATCCTGAAAAGGT
GAAGGTCTGGTACGAGAGGTCCCTTGTTCTGCAAAAGGAGGCAGACTCACTTTGTACTTTCATAGA
TGATTTGAAGCTGGCGATAGCACGAGAGAGTGATGGTAAAGACGCGAAAGTGAACGACATACGA
CGCAAAGATAACCTTGACGCTTCAAGTGTCGTGATGCTGAACCCAATCAACGGAAAAGGCTCAAC
CCTTCGGAAGGAAGTGGATAAGTTTCGGGAGCTTGTAGCTACGTTGATGACGGACAAGGCCAAGC
TCAAGTTGATTGAACAGGCACTGAATACTGAAAGCGGAACGAAGGGTAAGAGCTGGGAGTCCTCA
CTGTTCGAGAATATGCCAACAGTTGCCGCGATTACGCTCCTGACGAAGCTCCAGTCAGACGTACGG
TACGCGCAAGGTGAGGTACTTGCTGATCTTGTAAAAGGGAGCGGAACTaccggtTTGGAAGTGCTTTT
CCAGGGGCCTgCCGCGGccTCTAATTCCGCTGACGGTGACGGTTCAAATGCTACAGGGAGtTCTGCT
GGTGCTGGCTCTGGAACGAGTGGCGGGGACAACACGAGTGATGGCTCCGGGGCGAGTGCCGGTGC
AGCCAGCACAAATTCAAATGGGAACACGGGTAGTGCGACTTCTGGGGGGGCCACAGGTAGCGATA
CGTCAGGAGCGACGGCTGGTAGTGGGGCTTCCGACGGCGGAAACGGCGCAACAGCGTCATCAACT
ACAGGCAACGGAAATTCAAGCGGTACAACCGCGACGACCGGAGGCGGTGATGCAGGGggGTCGAC
tAATGCTGTGGGCCAGGACACGCAGGAGGTCATCGTGGTGCCACACTCCTTGCCCTTTAAGGTGGT
GGTGATCTCAGCCATCCTGGCCCTGGTGGTGCTCACCATCATCTCCCTTATCATCCTCATCATGCTT
TGGCAGAAGAAGCCACGTTAG²⁶⁸⁸gcgcgcaataatgccggctacttgctttaaaaaacctcccacacctccccctgaacctgaaacataaaa
tgaatgcaattgttgttgtt²⁷⁷⁷aacttgtttattgcagcttataatggttacaaataaagcaatagcatcacaaatttcacaaataaagcatttt
tttcactgcattctagttgtggtagtccaaactcatcaatgtatctta²⁸⁹⁹ACGCGTttcgaaTTAATTAA²⁹¹⁹AGGTTCCCAGGTGGGACACAAA
AACCGCAGAAAGGATCGTCGACCCTGAGGCCCAGGGCCCCTGGGCTGCCCTGTGGTCCAGTCTGAGGCCC
TTTCAGCCCCCAGGCTGCCTTGCCACCAGCTCCAGGTGCTCAAGATTCTGGCAGAGCCTGGACTCA
GGATGACTTGGAACTAGGGCTTGGCTCTCAGAAGTCCTGGATTTTGGAAACTCCAAATGGAATCAC
CCTTCAGAGACATCCCTGGTGCCTGGAGATGGGAATGTGGCCTCAGTGCCTCTGAGTAGGTGCCAT
GAGGCACCTTTGCTTTCTGCCCAGAGTGGCCATGAGCACCAGAACAGATGATCTCCATTTCCGCCA
GCTGCCTGTAGCCACGTGGCATCCTGCCTGTGGTCTGGGTGAGATTTACTGTGACCAGATGTAGAA
TAAATGTGTCTCATCCTGCATTTTTTTTCTAGAAACTGTTTCATAGTCTGCCCCCTCCAGGGGTAAG
AACAGTGTGCAGTTGTTGGCAGCAGTGGCCTGACCTCTTCCTGTCTAACTCCTTACATCCAGTCCA
GGGCATATCATAAGGCTTTGCCCATAGGACAGGCTTTGGAACTTGCCCGGGAGCACCCACCTGTG³⁵³⁹
CCGGCGAGGTTCCCAGGTGGGAC

### Sequence Annotation

| No. | Component sequences for RRP12_mCherry_SurfaceDisplay(porM) | Location (Residues) |
|---|---|---|
| 1. | sgRNA target sequence | 1-23 |
| 2. | Left homology arm (LHA) + sgRNA without PAM + reoptimized ORF | 24-695 |
| 3. | Glycine linker | 705-818 |
| 4. | mCherry | 870-1577 |
| 5. | P2A peptide | 1596-1652 |
| 6. | Surface display sequence (epitope: porM) | 1653-2687 |
| 7. | SV40 polyA signal | 2777-2898 |
| 8. | Right homology arm (RHA) | 2919-3538 |
| 9. | sgRNA target sequence | 3539-3561 |

### 2. RRP12_mCherry_SurfaceDisplay(STAS)

(SEQ ID NO: 13)
¹CCGGCGAGGTTCCCAGGTGGGAC²⁴CCCAGGATGGTCTTGATCCCCTGACCTTGTGATCTGCCCACC
TCGGCCTCCCAAAGTGCTGGGATTACAGGCATGAGCCACCACGCCCAGCCATAGTCATCATTTTTA
ATAGCTTTGTATAATTTGCTTTTCTAATCCCTTTATTGGTAGGAAATTAGAGTTGTTTCCGACTTTG
GCCCTTAAATTGGGTTATGTGTAGGACTGCTTTGGAAACTAATGTTACTAGGGAAATGGTGTTGTA
AAGTTCTAGCTTCTGCGGGTTGTAAGTTACCTTTCAATGGAGGGATGGGTGGGCAGAGGGAGCTTT
GACCTTCTCTGGACATACATTAGAGGAAAAATGGAAGGGAGGCCTGTTTCCAGGGGGATAATTGT
GCCAAAGTGGAATGTCCAGGTCAGGACATGAGCCGTGTGGAAGCTGGAACCACGTGAGGTCTGCC
TAGTTCATGTGCTGGCCACCACCTGGAGGCCCCCTTCTCATCCCTGCTGGCGCTGGGGGTGAGCCA
TCATTTGGCAACAGGAGGGGGCCTCCTATTCTCAGCCAGATGTGACCCTTCCGTTCCTTGGCCCTG
CAGGAAGAAGATGAAGCTGCAGGGACAGTTCAAAGGCCTGGTGAAGGCTGCtCGGCGAGGTTCCC
AGGTGGGACACAAAAATCGCCGGAAAGATAGAAGACCC⁶⁹⁶gcggccgcc⁷⁰⁵GGGGGCACGGGAAGTGG
TGGATCAGCCGGTGGCACTGGTGGCTCTGCCGGAGGGTCAGCGGGAGCAGGGGGAGCCACAGGC
GGATCTACGGCTGGAGGGGCGACAACGGCCTCT⁸¹⁹gcgatcgctGGCGAAAATCTGTATTTTCAGGGAG
GAgCTAGCGGAAGCGGA⁸⁷⁰ATGGTCAGTAAGGGTGAGGAGGACAACATGGCTATAATCAAAGAGT
TTATGCGGTTTAAGGTCCATATGGAAGGTTCAGTTAATGGACATGAGTTCGAGATAGAAGGTGAG
GGTGAGGGGCGACCGTACGAAGGCACACAAACCGCAAAGTTGAAAGTCACCAAAGGTGGACCCT
TGCCCTTTGCTTGGGATATTCTCTCCCCTCAATTCATGTACGGCAGTAAGGCATACGTCAAACATCC
CGCTGACATCCCCGACTATCTGAAGCTGTCTTTCCCTGAGGGTTTTAAATGGGAGCGAGTGATGAA
CTTCGAGGACGGGGGAGTGGTAACAGTGACTCAAGATTCCTCTTTGCAGGACGGGGAGTTCATAT
ATAAAGTGAAACTGCGGGGTACGAACTTTCCAAGTGACGGtCCCGTAATGCAGAAGAAGACGATG
GGATGGGAGGCAAGCAGCGAGCGAATGTATCCTGAGGATGGAGCCCTTAAGGGAGAAATTAAGC
AACGGCTGAAGTTGAAAGATGGTGGACATTATGATGCTGAGGTTAAAACAACTTATAAAGCCAAG
AAACCAGTTCAGTTGCCAGGGGCGTATAACGTCAACATTAAACTGGACATTACATCTCACAATGA
AGATTACACAATCGTTGAGCAATATGAaCGCGCGGAGGGTCGGCACTCAACGGGTGGCATGGACG
AGTTGTATAAA¹⁵⁷⁸GGcgcgcccggaagcgga¹⁵⁹⁶gctactaacttcagcctgctgaagcaggctggagacgtggaggagaaccctggacct¹⁶⁵³
atgggctggtcatgtatcattctgtttctggtcgcaaccgcaactggagtgcattcacaggtgcagctcggcggaccgTCCCAACTGAGCCAA
GTAACGCCAGTGGATGAAGTGGACGGAACCAGAACGTATCGCGTTCGGGGGCAACTCTTTTTCGTCTCT
ACCCATGACTTCTTGCACCAGTTCGACTTTACCCATCCAGCAAGGCGGGTGGTGATTGACCTCTCT
GACGCTCACTTTTGGGATGGGAGTGCCGTAGGAGCTTTGGACAAGGTGATGCTGAAGTTTATGAG
ACAGGGCACGAGTGTCGAGCTGCGCGGGCTGAACGCTGCAAGTGCCACTCTTGTTGAACGGCTTG
GGAGCGGAACTaccggtGGCGAAAATCTGTATTTTCAGGGAgCCGCGGccTCTAATTCCGCTGACGGTG
ACGGTTCAAATGCTACAGGGAGtTCTGCTGGTGCTGGCTCTGGAACGAGTGGCGGGGACAACACGA
GTGATGGCTCCGGGGCGAGTGCCGGTGCAGCCAGCACAAATTCAAATGGGAACACGGGTAGTGCG
ACTTCTGGGGGGGCCACAGGTAGCGATACGTCAGGAGCGACGGCTGGTAGTGGGGCTTCCGACGG
CGGAAACGGCGCAACAGCGTCATCAACTACAGGCAACGGAAATTCAAGCGGTACAACCGCGACG
ACCGGAGGCGGTGATGCAGGGggGTCGACtAATGCTGTGGGCCAGGACACGCAGGAGGTCATCGTG
GTGCCACACTCCTTGCCCTTTAAGGTGGTGGTGATCTCAGCCATCCTGGCCCTGGTGGTGCTCACC
ATCATCTCCCTTATCATCCTCATCATGCTTTGGCAGAAGAAGCCACGTTAG²⁵²³gcgcgcaataatgccggctact
tgctttaaaaaacctcccacacctccccctgaacctgaaacataaaatgaatgcaattgttgttgtt²⁶¹²aacttgtttattgcagcttataa
tggttacaaataaagcaatagcatcacaaatttcacaaataaagcatttttttcactgcattctagttgtggtttgtccaaactcatcaatg
tatctta²⁷³⁴ACGCGTttcgaaTTAATTAA²⁷⁵⁴AGGTTCCCAGGTGGGACACAAAAACCGCAGAAAGGATCGTCGACCCTGAGGCCCAGGGCC
CCTGGGCTGCCCTGTGGTCCAGTCTGAGGCCCTTTCAGCCCCCAGGCTGCCTTGCCACCAGCTCCAGGTG
CTCAAGATTCTGGCAGAGCCTGGACTCAGGATGACTTGGAACTAGGGCTTGGCTCTCAGAAGTCCT
GGATTTTGGAAACTCCAAATGGAATCACCCTTCAGAGACATCCCTGGTGCCTGGAGATGGGAATGT
GGCCTCAGTGCCTCTGAGTAGGTGCCATGAGGCACCTTTGCTTTCTGCCCAGAGTGGCCATGAGCA
CCAGAACAGATGATCTCCATTTCCGCCAGCTGCCTGTAGCCACGTGGCATCCTGCCTGTGGTCTGG
GTGAGATTTACTGTGACCAGATGTAGAATAAATGTGTCTCATCCTGCATTTTTTTTCTAGAAACTGT
TTCATAGTCTGCCCCCTCCAGGGGTAAGAACAGTGTGCAGTTGTTGGCAGCAGTGGCCTGACCTCT
TCCTGTCTAACTCCTTACATCCAGTCCAGGGCATATCATAAGGCTTTGCCCATAGGACAGGCTTTG
GAACTTGCCCGGGAGCACCCACCTGTG³³⁷⁴CCGGCGAGGTTCCCAGGTGGGAC

### Sequence Annotation

| No. | Component sequences for RRP12_mCherry_SurfaceDisplay(STAS) | Location (Residues) |
|---|---|---|
| 1. | sgRNA target sequence | 1-23 |
| 2. | Left homology arm (LHA) + sgRNA without PAM + reoptimized ORF | 24-695 |
| 3. | Glycine linker | 705-818 |
| 4. | MCherry | 870-1577 |
| 5. | P2A peptide | 1596-1652 |
| 6. | Surface display sequence (epitope: STAS) | 1653-2522 |
| 7. | SV40 polyA signal | 2612-2733 |
| 8. | Right homology arm (RHA) | 2754-3373 |
| 9. | sgRNA target sequence | 3374-3396 |

### 3. 3. Pes1_mCherry_SurfaceDisplay(porM)

(SEQ ID NO: 14)
¹CCCACGATGAGGCGGTGAGGTCT²⁴GACCAGCGTTGGCAACATATTGAGACCCTGTCTCTACCCCC
CAAAAAAAAAAAGAAAGGGCTACGCATGGTGGTGCACACCTGTAGTCAATCCCAGCTACTCCGGA
GGCTGAAGTGGGAGGATCGTTTGAGGCTGCAGTGAGCTATGATTGTGCCACTGTGCTCCAGGCTGA
GCAACAGAGAAAGACCCTGTCCCTTTAAAAAAATTAAAAATATATTGTCAGATGACCCCGGAAAG
AAGGTTCTTCCTGTTGTACCCCTTTCCACCAGCTCCTGGTGAAGGTTCTAGTGGCATCCAGCTTTCC
CAGGTGGTGTAGGGAAATGGGGCAGTTGCCAAGGCTCCTTCCAGCTCTGGGAGTTTAGGATTCTCT
TATCTCGAGATTTGTGGGCCCATGAAATAATGTTGTTAAAGCAGGGCTAGCGCATGTTTTCTCACC
ATGAAGTGGGTCAGGTAGATTTTTTTCCTGTGAGAATTTGTGACCTTTTCTTGAAGCTCTGCTTTTA
AGGGATATAGCTTTGAGTTCTGTGCCCCCCACCCTCCCTTCTACACATACCTCAGCCTGACCTTCGC
CTTCCCCCTCACAGGCCAACAAGCTGGCGGAGAAGCGGAAAGCACACGATGAGGCTGTAAGATCA
GAGAAGAAGGCGAAAAAGGCGCGACCTGAG⁶⁸⁹GCggccgcc⁶⁹⁸GGGGGCACGGGAAGTGGTGGATCA
GCCGGTGGCACTGGTGGCTCTGCCGGAGGGTCAGCGGGAGCAGGGGGAGCCACAGGCGGATCTAC
GGCTGGAGGGGCGACAACGGCCTCT⁸¹²gcgatcgctGGCGAAAATCTGTATTTTCAGGGAGGAgCTAGC
GGAAGCGGA⁸⁶³ATGGTCAGTAAGGGTGAGGAGGACAACATGGCTATAATCAAAGAGTTTATGCGG
TTTAAGGTCCATATGGAAGGTTCAGTTAATGGACATGAGTTCGAGATAGAAGGTGAGGGTGAGGG
GCGACCGTACGAAGGCACACAAACCGCAAAGTTGAAAGTCACCAAAGGTGGACCCTTGCCCTTTG
CTTGGGATATTCTCTCCCCTCAATTCATGTACGGCAGTAAGGCATACGTCAAACATCCCGCTGACA
TCCCCGACTATCTGAAGCTGTCTTTCCCTGAGGGTTTTAAATGGGAGCGAGTGATGAACTTCGAGG
ACGGGGGAGTGGTAACAGTGACTCAAGATTCCTCTTTGCAGGACGGGGAGTTCATATATAAAGTG
AAACTGCGGGGTACGAACTTTCCAAGTGACGGtCCCGTAATGCAGAAGAAGACGATGGGATGGGA
GGCAAGCAGCGAGCGAATGTATCCTGAGGATGGAGCCCTTAAGGGAGAAATTAAGCAACGGCTG
AAGTTGAAAGATGGTGGACATTATGATGCTGAGGTTAAAACAACTTATAAAGCCAAGAAACCAGT
TCAGTTGCCAGGGGCGTATAACGTCAACATTAAACTGGACATTACATCTCACAATGAAGATTACAC
AATCGTTGAGCAATATGAaCGCGCGGAGGGTCGGCACTCAACGGGTGGCATGGACGAGTTGTATA
AA¹⁵⁷¹GGcgcgcccggaagcgga¹⁵⁸⁹gctactaacttcagcctgctgaagcaggctggagacgtggaggagaaccctggacct¹⁶⁴⁶atgggct
ggtcatgtatcattctgtttctggtcgcaaccgcaactggagtgcattcacaggtgcagctcggcggaccgACGAATCCTGAAAAGGTGAAG
GTCTGGTACGAGAGGTCCCTTGTTCTGCAAAAGGAGGCAGACTCACTTTGTACTTTCATAGATGATTTGAA
GCTGGCGATAGCACGAGAGAGTGATGGTAAAGACGCGAAAGTGAACGACATACGACGCAAAGAT
AACCTTGACGCTTCAAGTGTCGTGATGCTGAACCCAATCAACGGAAAAGGCTCAACCCTTCGGAA
GGAAGTGGATAAGTTTCGGGAGCTTGTAGCTACGTTGATGACGGACAAGGCCAAGCTCAAGTTGA
TTGAACAGGCACTGAATACTGAAAGCGGAACGAAGGGTAAGAGCTGGGAGTCCTCACTGTTCGAG
AATATGCCAACAGTTGCCGCGATTACGCTCCTGACGAAGCTCCAGTCAGACGTACGGTACGCGCA
AGGTGAGGTACTTGCTGATCTTGTAAAAGGGAGCGGAACTaccggtTTGGAAGTGCTTTTCCAGGGGC
CTgCCGCGGccTCTAATTCCGCTGACGGTGACGGTTCAAATGCTACAGGGAGtTCTGCTGGTGCTGG
CTCTGGAACGAGTGGCGGGGACAACACGAGTGATGGCTCCGGGGCGAGTGCCGGTGCAGCCAGCA
CAAATTCAAATGGGAACACGGGTAGTGCGACTTCTGGGGGGGCCACAGGTAGCGATACGTCAGGA
GCGACGGCTGGTAGTGGGGCTTCCGACGGCGGAAACGGCGCAACAGCGTCATCAACTACAGGCAA
CGGAAATTCAAGCGGTACAACCGCGACGACCGGAGGCGGTGATGCAGGGggGTCGACtAATGCTGT
GGGCCAGGACACGCAGGAGGTCATCGTGGTGCCACACTCCTTGCCCTTTAAGGTGGTGGTGATCTC
AGCCATCCTGGCCCTGGTGGTGCTCACCATCATCTCCCTTATCATCCTCATCATGCTTTGGCAGAAG
AAGCCACGTTAG²⁶⁸¹gcgcgcaataatgccggctacttgctttaaaaaacctcccacacctccccctgaacctgaaacataaaatgaatgcaat
tgttgttgtt²⁷⁷⁰aacttgatattgcagcttataatggttacaaataaagcaatagcatcacaaatttcacaaataaagcatttttacactgca
ttctagttgtggtttgtccaaactcatcaatgtatctta²⁸⁹²ACGCGTttcgaaTTAATTAAATGAGGCGGTGAGGTCT²⁹²⁹GAGAAGAAGGCC
AAGAAGGCAAGGCCGGAGTGAGTGCCTGCGGCCCCTCACAGGGCTGAGGCCAGCCCCTAGCAGCTGGATGTG
GCAGAGGCAGGCCAGAGGACCTAAGTGTGATGGACCAGAGTCACTTCTCCTCCTCCTTTCTCCAGC
CAGCCCTGACCCCTCATGCTCTCTGGCTGGGCCAGTGGGCAGCCCTCGCTTCCCTTGGATGGAGCT
GCCCTGCTGGTGCCTGGTCAGAGAAGAGGCCTCTGTGCCCAGCCTGATTCTCTGCTCCCAGGAGCC
AGTGACATGAGGTGCAGAGGCCCACCCAGCCCCCTACCTACTGCCCCCATTCATCCTGGCTTTCCA
CAGCCCCCTCCCACACAGTTGGACCCGTGATTCTCAGGGTGCTGTGATGGGGTGAGGGTAGGGGG
AGCATTTGTTATTAAATGACTGGACTTTTGTGCCAATTGCATTTTGTGTCCATGAGCCTTCCTAGGG
TTGGAGGAGGCCTACCTAGCACTCTATGCTGCAGGCTGGGCCAGCCCTGGGTATTTACTGAGACAG
AGCTGGGCACTGCTCAGAGCTCTCTGGATGTCCAAGGACCCCTCCAGGTCCAGGGATGCCAAAAG
GTAGGTGCA³⁵⁴⁹CCCACGATGAGGCGGTGAGGTCT

### Sequence Annotation

| No. | Component sequences for Pes1_mCherry_SurfaceDisplay(porM) | Location (Residues) |
|---|---|---|
| 1. | sgRNA target sequence | 1-23 |
| 2. | Left homology arm (LHA) + sgRNA without PAM + reoptimized ORF | 24-688 |
| 3. | Glycine linker | 698-811 |
| 4. | MCherry | 863-1570 |
| 5. | P2A peptide | 1589-1645 |
| 6. | Surface display sequence (epitope: porM) | 1646-2680 |
| 7. | SV40 polyA signal | 2770-2891 |
| 8. | Right homology arm (RHA) | 2929-3548 |
| 9. | sgRNA target sequence | 3549-3571 |

### 4. Pes1_mCherry_SurfaceDisplay(STAS)

(SEQ ID NO: 15)
¹CCCACGATGAGGCGGTGAGGTCT²⁴GACCAGCGTTGGCAACATATTGAGACCCTGTCTCTACCCCC
CAAAAAAAAAAAGAAAGGGCTACGCATGGTGGTGCACACCTGTAGTCAATCCCAGCTACTCCGGA
GGCTGAAGTGGGAGGATCGTTTGAGGCTGCAGTGAGCTATGATTGTGCCACTGTGCTCCAGGCTGA
GCAACAGAGAAAGACCCTGTCCCTTTAAAAAAATTAAAAATATATTGTCAGATGACCCCGGAAAG
AAGGTTCTTCCTGTTGTACCCCTTTCCACCAGCTCCTGGTGAAGGTTCTAGTGGCATCCAGCTTTCC
CAGGTGGTGTAGGGAAATGGGGCAGTTGCCAAGGCTCCTTCCAGCTCTGGGAGTTTAGGATTCTCT
TATCTCGAGATTTGTGGGCCCATGAAATAATGTTGTTAAAGCAGGGCTAGCGCATGTTTTCTCACC
ATGAAGTGGGTCAGGTAGATTTTTTTCCTGTGAGAATTTGTGACCTTTTCTTGAAGCTCTGCTTTTA
AGGGATATAGCTTTGAGTTCTGTGCCCCCCACCCTCCCTTCTACACATACCTCAGCCTGACCTTCGC
CTTCCCCCTCACAGGCCAACAAGCTGGCGGAGAAGCGGAAAGCACACGATGAGGCTGTAAGATCA
GAGAAGAAGGCGAAAAAGGCGCGACCTGAG⁶⁸⁹GCggccgcc⁶⁹⁸GGGGGCACGGGAAGTGGTGGATCA
GCCGGTGGCACTGGTGGCTCTGCCGGAGGGTCAGCGGGAGCAGGGGGAGCCACAGGCGGATCTAC
GGCTGGAGGGGCGACAACGGCCTCT⁸¹²gcgatcgctGGCGAAAATCTGTATTTTCAGGGAGGAgCTAGC
GGAAGCGGA⁸⁶³ATGGTCAGTAAGGGTGAGGAGGACAACATGGCTATAATCAAAGAGTTTATGCGG
TTTAAGGTCCATATGGAAGGTTCAGTTAATGGACATGAGTTCGAGATAGAAGGTGAGGGTGAGGG
GCGACCGTACGAAGGCACACAAACCGCAAAGTTGAAAGTCACCAAAGGTGGACCCTTGCCCTTTG
CTTGGGATATTCTCTCCCCTCAATTCATGTACGGCAGTAAGGCATACGTCAAACATCCCGCTGACA
TCCCCGACTATCTGAAGCTGTCTTTCCCTGAGGGTTTTAAATGGGAGCGAGTGATGAACTTCGAGG
ACGGGGGAGTGGTAACAGTGACTCAAGATTCCTCTTTGCAGGACGGGGAGTTCATATATAAAGTG
AAACTGCGGGGTACGAACTTTCCAAGTGACGGtCCCGTAATGCAGAAGAAGACGATGGGATGGGA
GGCAAGCAGCGAGCGAATGTATCCTGAGGATGGAGCCCTTAAGGGAGAAATTAAGCAACGGCTG
AAGTTGAAAGATGGTGGACATTATGATGCTGAGGTTAAAACAACTTATAAAGCCAAGAAACCAGT
TCAGTTGCCAGGGGCGTATAACGTCAACATTAAACTGGACATTACATCTCACAATGAAGATTACAC
AATCGTTGAGCAATATGAaCGCGCGGAGGGTCGGCACTCAACGGGTGGCATGGACGAGTTGTATA
AA¹⁵⁷¹GGcgcgcccggaagcgga¹⁵⁸⁹gctactaacttcagcctgctgaagcaggctggagacgtggaggagaaccctggacct¹⁶⁴⁶atgggctg
gtcatgtatcattctgtttctggtcgcaaccgcaactggagtgcattcacaggtgcagctcggcggaccgTCCCAACTGAGCCAAGTAACGCC
AGTGGATGAAGTGGACGGAACCAGAACGTATCGCGTTCGGGGGCAACTCTTTTTCGTCTCTACCCATGAC
TTCTTGCACCAGTTCGACTTTACCCATCCAGCAAGGCGGGTGGTGATTGACCTCTCTGACGCTCACT
TTTGGGATGGGAGTGCCGTAGGAGCTTTGGACAAGGTGATGCTGAAGTTTATGAGACAGGGCACG
AGTGTCGAGCTGCGCGGGCTGAACGCTGCAAGTGCCACTCTTGTTGAACGGCTTGGGAGCGGAAC
TaccggtGGCGAAAATCTGTATTTTCAGGGAgCCGCGGccTCTAATTCCGCTGACGGTGACGGTTCAAA
TGCTACAGGGAGtTCTGCTGGTGCTGGCTCTGGAACGAGTGGCGGGGACAACACGAGTGATGGCTC
CGGGGCGAGTGCCGGTGCAGCCAGCACAAATTCAAATGGGAACACGGGTAGTGCGACTTCTGGGG
GGGCCACAGGTAGCGATACGTCAGGAGCGACGGCTGGTAGTGGGGCTTCCGACGGCGGAAACGG
CGCAACAGCGTCATCAACTACAGGCAACGGAAATTCAAGCGGTACAACCGCGACGACCGGAGGC
GGTGATGCAGGGggGTCGACtAATGCTGTGGGCCAGGACACGCAGGAGGTCATCGTGGTGCCACAC
TCCTTGCCCTTTAAGGTGGTGGTGATCTCAGCCATCCTGGCCCTGGTGGTGCTCACCATCATCTCCC
TTATCATCCTCATCATGCTTTGGCAGAAGAAGCCACGTTAG²⁵¹⁶gcgcgcaataatgccggctacttgctttaaaaaacctc
ccacacctccccctgaacctgaaacataaaatgaatgcaattgttgttgtt²⁶⁰⁵aacttgatattgcagcttataatggttacaaataaagcaa
tagcatcacaaatttcacaaataaagcatttttttcactgcattctagttgtggtttgtccaaactcatcaatgtatctta²⁷²⁷ACGCGTttc
gaaTTAATTAAATGAGGCGGTGAGGTCT²⁷⁶⁴GAGAAGAAGGCCAAGAAGGCAAGGCCGGAGTGAGTGCCTGCGGCCCCTCACAGGG
CTGAGGCCAGCCCCTAGCAGCTGGATGTGGCAGAGGCAGGCCAGAGGACCTAAGTGTGATGGACC
AGAGTCACTTCTCCTCCTCCTTTCTCCAGCCAGCCCTGACCCCTCATGCTCTCTGGCTGGGCCAGTG
GGCAGCCCTCGCTTCCCTTGGATGGAGCTGCCCTGCTGGTGCCTGGTCAGAGAAGAGGCCTCTGTG
CCCAGCCTGATTCTCTGCTCCCAGGAGCCAGTGACATGAGGTGCAGAGGCCCACCCAGCCCCCTAC
CTACTGCCCCCATTCATCCTGGCTTTCCACAGCCCCCTCCCACACAGTTGGACCCGTGATTCTCAGG
GTGCTGTGATGGGGTGAGGGTAGGGGGAGCATTTGTTATTAAATGACTGGACTTTTGTGCCAATTG
CATTTTGTGTCCATGAGCCTTCCTAGGGTTGGAGGAGGCCTACCTAGCACTCTATGCTGCAGGCTG
GGCCAGCCCTGGGTATTTACTGAGACAGAGCTGGGCACTGCTCAGAGCTCTCTGGATGTCCAAGG
ACCCCTCCAGGTCCAGGGATGCCAAAAGGTAGGTGCA³³⁸⁴CCCACGATGAGGCGGTGAGGTCT

### Sequence Annotation

| No. | Component sequences for Pes1_mCherry_SurfaceDisplay(STAS) | Location (Residues) |
|---|---|---|
| 1. | sgRNA target sequence | 1-23 |
| 2. | Left homology arm (LHA) + sgRNA without PAM + reoptimized ORF | 24-688 |
| 3. | Glycine linker | 698-811 |
| 4. | mCherry | 863-1570 |
| 5. | P2A peptide | 1589-1645 |
| 6. | Surface display sequence (epitope: porM) | 1646-2680 |
| 7. | SV40 polyA signal | 2770-2891 |
| 8. | Right homology arm (RHA) | 2929-3548 |
| 9. | sgRNA target sequence | 3549-3571 |

### 5. Noc3L_GFP_SurfaceDisplay(BtuF)

(SEQ ID NO: 16)
¹AGTTGCTACTGAATCGCCTCTGG²⁴TGGATTGGTTGGTTAGTTTCAAATCTTATACCTTAATATATG
GGTTAAGAATGAATCATTCTCTGAGTATAATCTAATTATTTTTGAGTTACACAGATGTGGTGGTATC
TTTACATTTTTTGTGTTTGTGATTTAGATCTGCTACTGAACTTTTTGAGGCATATAGCATGGCAGAA
ATGACATTCAATCCTCCTGTTGAATCTTCAAACCCCAAAATAAAGGTATGGGATATTTTTCATTTTT
TTAAAGGAAGAAATAGAAACCAATGTATCTCAATAACTCTAACTCCAGTTTGCTTAATTATTTTAT
AGGTAGTTTTTTTTTTAATGTTTAGGATTTCATCATAGGATGGATTTCTGAGGTTGAAATTCTATAG
AGATGATCATGAAACTGTTCGTTCAATATAGGATATGTCCAAGACCTTACCAAGCATCTGTCATTG
TGTTGCATGTGTTGGTGTCAGCTGTTGCCATTTTCAACTTGGTTCACAGGTTGGCTTTAGCTTATAG
CATAAGTAACTTCTAACTCATACTTTAAATATTTTCCTAGGGTAAATTTTTACAAGGGGATTCATTT
TTGAATGAAGATTTAAATCAGCTAATCAAAAGATACTCCAGTGAAGTTGCTACTGAATCGCCTCTT
GACTTTACCAAGTACCTCAAGACAAGTCTTCAC⁶⁹⁹gcggccgcc⁷⁰⁸GGGGGCACGGGAAGTGGTGGATC
AGCCGGTGGCACTGGTGGCTCTGCCGGAGGGTCAGCGGGAGCAGGGGGAGCCACAGGCGGATCT
ACGGCTGGAGGGGCGACAACGGCCTCT⁸²²gcgatcgctTTGGAAGTGCTTTTCCAGGGGCCTGGAgCTAG
CGGAAGCGGA⁸⁷³GGATCAAAGGGAGAGGAACTCTTTACCGGCGTCGTTCCAATCCTTGTTGAACTG
GATGGGGACGTGAATGGGCATAAATTTTCAGTATCAGGGGAAGGGGAAGGCGACGCTACATATGG
AAAATTGACTCTCAAATTCATATGCACTACTGGTAAATTGCCCGTGCCTTGGCCTACACTCGTCAC
GACCTTCGGGTATGGTGTTCAATGTTTCGCCAGGTATCCGGATCATATGAAACAACACGATTTCTT
CAAATCAGCGATGCCGGAAGGGTATGTGCAGGAGCGAACAATCTTTTTCAAGGACGACGGCAACT
ATAAAACACGGGCCGAAGTCAAATTTGAGGGAGATACGCTCGTTAATCGGATAGAGCTGAAGGGC
ATCGACTTTAAGGAGGATGGGAACATCTTGGGCCATAAGCTGGAATATAATTATAACAGCCACAA
CGTTTACATTATGGCCGACAAACAGAAGAATGGTATTAAGGTGAATTTTAAAATAAGGCACAACA
TAGAAGACGGATCTGTGCAACTGGCCGACCACTATCAGCAGAATACGCCTATTGGCGATGGTCCA
GTGCTTCTCCCTGACAACCATTACCTCAGTACGCAAAGTGCTCTCTCTAAAGACCCCAACGAAAAA
CGCGATCACATGGTACTGCTGGAGTTCGTAACCGCCGCAGGAATAACTCATGGAATGGATGAACT
CTACAAGGTTGACTTGGATAAA¹⁶⁰²GGCGCGCCCG¹⁶¹²gaagttcctattctctagaaagtataggaacttc¹⁶⁴⁶GGGGTCTG
GC¹⁶⁵⁶GAAGGCAGAGGCTCCCTTTTGACATGcGGAGACGTCGAGGAGAACCCGGGTCCC¹⁷¹⁰ATGGA
GACAGACACACTCCTGCTATGGGTACTGCTcCTCTGGGTtCCAGGTTCCACTGGcGACggcggaccgACC
GCCAACACCTCCTCCACCTCCACCAACGGCAACGCTGCGCCACGGGTTATTACCCTTTCACCTGCG
AACACAGAATTGGCCTTCGCAGCGGGGATCACGCCGGTTGGCGTTAGTAGCTATTCAGATTATCCG
CCACAGGCACAAAAAATCGAGCAAGTCTCAACTTGGCAGGGTATGAACCTGGAACGCATAGTGGC
TTTGAAGCCCGACCTGGTTATCGCTTGGCGGGGCGGGAATGCCGAGAGGCAGGTTGATCAGTTGG
CCTCCCTGGGTATAAAAGTAATGTGGGTGGATGCAACAAGTATTGAACAAATAGCAAATGCCTTG
AGACAGTTGGCCCCGTGGAGTCCCCAGCCTGACAAAGCTGAACAAGCTGCTCAAAGCCTTCTTGA
CCAGTATGCACAGTTGAAAGCGCAATACGCAGATAAGCCTAAGAAGCGCGTATTTTTGCAATTTG
GAATTAATCCTCCATTTACCTCTGGTAAGGAGTCAATTCAAAATCAAGTCTTGGAGGTCTGTGGAG
GGGAGAATATTTTTAAGGATAGTAGGGTCCCCTGGCCCCAGGTAAGCCGAGAACAAGTGCTGGCC
CGGAGTCCACAGGCAATCGTCATCACAGGGGGACCCGACCAAATTCCCAAGATCAAACAGTACTG
GGGGGAGCAACTCAAAATTCCAGTCATACCACTGACATCAGACTGGTTCGAACGGGCaAGCCCCC
GGATCATACTCGCTGCACAACAACTCTGCAAtGCGTTGAGCCAGGTTGACGGAGGAAACTCCTCCA
ACTCCGCCACCAACACCTCCGCCACCaccggtTTGGAAGTGCTTTTCCAGGGGCCTgCCGCGGccTCTA
ATTCCGCTGACGGTGACGGTTCAAATGCTACAGGGAGtTCTGCTGGTGCTGGCTCTGGAACGAGTG
GCGGGGACAACACGAGTGATGGCTCCGGGGCGAGTGCCGGTGCAGCCAGCACAAATTCAAATGG
GAACACGGGTAGTGCGACTTCTGGGGGGGCCACAGGTAGCGATACGTCAGGAGCGACGGCTGGTA
GTGGGGCTTCCGACGGCGGAAACGGCGCAACAGCGTCATCAACTACAGGCAACGGAAATTCAAGC
GGTACAACCGCGACGACCGGAGGCGGTGATGCAGGGggGTCGACtAATGCTGTGGGCCAGGACACG
CAGGAGGTCATCGTGGTGCCACACTCCTTGCCCTTTAAGGTGGTGGTGATCTCAGCCATCCTGGCC
CTGGTGGTGCTCACCATCATCTCCCTTATCATCCTCATCATGCTTTGGCAGAAGAAGCCACGTTAG³⁰⁹⁶
GCGCGCAATAATG³¹⁰⁹gaagttcctattctctagaaagtataggaacttc³¹⁴³GTAAGccggctacttgctttaaaaaacctcccacacctccc
cctgaacctgaaacataaaatgaatgcaattgttgttgtt³²²⁴aacttgtttattgcagcttataatggttacaaataaagcaatagcatcaca
aatttcacaaataaagcatttttttcactgcattctagttgtggtttgtccaaactcatcaatgtatctta³³⁴⁶ACGCGTttcgaaTTAATTAA
CTCTGG³³⁷²ATTTCACGAAATATTTGAAAACATCACTACACTAGTAGAGGAATGAAGTCAGTGGACTTTCTTGTATATTTGTGTGT
GCAGATGTACATAAAGATGAGTTGTTAACTTAGGATCTTTTCTTTTTATACAAGGAAAGCTTCCTA
AGAATGTCTAGGAAGAAGAGGAAGAATGACCCTTTGCATGGCACAGGGTTCTGCCCCTATTCTGA
ATATGTCATTCCATCAAGGAGATCAAAAGCCTTTTTTTCTCCCCAGTATTTGGAAATTACTTTCTTG
ATGATGCTGCCTTTTAAAAGCTTCACGTACATTATAGTTTTTTAAAAAAATCTTTGGACTGGATCTT
ACTGAAGTGCAGTTGCTATATTAAAATTAGGGCATAGAGCACAGAAAAATCAAGACCATGAGAAG
ACATTTTACCATTTAGCTACTTTTTATAACTAAATACTCTTTAAATATTTTTATTTCAATACTGTGGA
TGGAAATGAGAAGCATTCTAAATTTGAGTTAATATATTTTTATGAAGATATTTGAGAAAAGAAAAA
AATAGCTTGTATTCAGGTTCATTGGCTTTTGCTGGATGATCCACCTAAAGAAGTTACCTAATTTGGC
CTTTTA³³⁸⁶AGTTGCTACTGAATCGCCTCTGG

### Sequence Annotation

| No. | Component sequences for Noc3L_GFP_SurfaceDisplay(BtuF) | Location (Residues) |
|---|---|---|
| 1. | sgRNA target sequence | 1-23 |
| 2. | Left homology arm (LHA) + sgRNA without PAM + reoptimized ORF | 24-698 |
| 3. | Glycine linker | 708-821 |
| 4. | Gfp | 873-1601 |
| 5. | 1^{st} FRT sequence for FLP-FRT recombination | 1612-1645 |
| 6. | T2A peptide | 1656-1709 |
| 7. | Surface display sequence (epitope: BtuF) | 1710-3095 |
| 8. | 2^{nd} FRT sequence for FLP-FRT recombination | 3109-3142 |
| 7. | SV40 polyA signal | 3224-3345 |
| 8. | Right homology arm (RHA) | 3372-3985 |
| 9. | sgRNA target sequence | 3986-4008 |

### 6. Noc3L_GFP_SurfaceDisplay(Hivp24)

(SEQ ID NO: 17)
¹AGTTGCTACTGAATCGCCTCTGG²⁴TGGATTGGTTGGTTAGTTTCAAATCTTATACCTTAATATATG
GGTTAAGAATGAATCATTCTCTGAGTATAATCTAATTATTTTTGAGTTACACAGATGTGGTGGTATC
TTTACATTTTTTGTGTTTGTGATTTAGATCTGCTACTGAACTTTTTGAGGCATATAGCATGGCAGAA
ATGACATTCAATCCTCCTGTTGAATCTTCAAACCCCAAAATAAAGGTATGGGATATTTTTCATTTTT
TTAAAGGAAGAAATAGAAACCAATGTATCTCAATAACTCTAACTCCAGTTTGCTTAATTATTTTAT
AGGTAGTTTTTTTTTTAATGTTTAGGATTTCATCATAGGATGGATTTCTGAGGTTGAAATTCTATAG
AGATGATCATGAAACTGTTCGTTCAATATAGGATATGTCCAAGACCTTACCAAGCATCTGTCATTG
TGTTGCATGTGTTGGTGTCAGCTGTTGCCATTTTCAACTTGGTTCACAGGTTGGCTTTAGCTTATAG
CATAAGTAACTTCTAACTCATACTTTAAATATTTTCCTAGGGTAAATTTTTACAAGGGGATTCATTT
TTGAATGAAGATTTAAATCAGCTAATCAAAAGATACTCCAGTGAAGTTGCTACTGAATCGCCTCTT
GACTTTACCAAGTACCTCAAGACAAGTCTTCAC⁶⁹⁹gcggccgcc⁷⁰⁸GGGGGCACGGGAAGTGGTGGATC
AGCCGGTGGCACTGGTGGCTCTGCCGGAGGGTCAGCGGGAGCAGGGGGAGCCACAGGCGGATCT
ACGGCTGGAGGGGCGACAACGGCCTC⁸²²gcgatcgctTTGGAAGTGCTTTTCCAGGGGCCTGGAgCTAG
CGGAAGCGGA⁸⁷³GGATCAAAGGGAGAGGAACTCTTTACCGGCGTCGTTCCAATCCTTGTTGAACTG
GATGGGGACGTGAATGGGCATAAATTTTCAGTATCAGGGGAAGGGGAAGGCGACGCTACATATGG
AAAATTGACTCTCAAATTCATATGCACTACTGGTAAATTGCCCGTGCCTTGGCCTACACTCGTCAC
GACCTTCGGGTATGGTGTTCAATGTTTCGCCAGGTATCCGGATCATATGAAACAACACGATTTCTT
CAAATCAGCGATGCCGGAAGGGTATGTGCAGGAGCGAACAATCTTTTTCAAGGACGACGGCAACT
ATAAAACACGGGCCGAAGTCAAATTTGAGGGAGATACGCTCGTTAATCGGATAGAGCTGAAGGGC
ATCGACTTTAAGGAGGATGGGAACATCTTGGGCCATAAGCTGGAATATAATTATAACAGCCACAA
CGTTTACATTATGGCCGACAAACAGAAGAATGGTATTAAGGTGAATTTTAAAATAAGGCACAACA
TAGAAGACGGATCTGTGCAACTGGCCGACCACTATCAGCAGAATACGCCTATTGGCGATGGTCCA
GTGCTTCTCCCTGACAACCATTACCTCAGTACGCAAAGTGCTCTCTCTAAAGACCCCAACGAAAAA
CGCGATCACATGGTACTGCTGGAGTTCGTAACCGCCGCAGGAATAACTCATGGAATGGATGAACT
CTACAAGGTTGACTTGGATAAA¹⁶⁰²GGCGCGCCCG¹⁶¹²gaagttcctattctctagaaagtataggaacttc¹⁶⁴⁶GGGGTCTG
GC¹⁶⁵⁶GAAGGCAGAGGCTCCCTTTTGACATGcGGAGACGTCGAGGAGAACCCGGGTCCC¹⁷¹⁰ATGGA
GACAGACACACTCCTGCTATGGGTACTGCTcCTCTGGGTtCCAGGTTCCACTGGcGACggcggaccgACC
GCCAACACCTCCTCCACCTCCACCAACGGCAACAGCATTTTGGACATACGCCAAGGCCCGAAAGA
GCCATTTCGCGATTACGTAGATCGGTTCTACAAAACGCTGCGAGCGGAGCAAGCATCACAAGAGG
TTAAAAATTGGATGACGGAGACATTGCTTGTTCAAAACGCGAACCCAGATTGTAAAACAATTTTGA
AAGCCCTTGGACCTGGTGCTACGCTCGAGGAAATGATGACAGCATGCCAAGGCGTTGGtGGaCCAG
GAGGAAGTACCGGAGGAAGCATCCTTGATATACGACAAGGTCCTAAGGAGCCTTTTCGCGACTAC
GTTGACCGCTTTTATAAGACGcttCGCGCTGAACAGGCGTCTCAGGAGGTCAAGAATTGGATGACAG
AGACATTGCTTGTACAAAATGCTAATCCCGACTGTAAAACGATTCTCAAGGCGCTGGGACCGGGA
GCCACTCTTGAAGAAATGATGACTGCGTGTCAAGGAGTAGGAGGAAACTCCTCCAACTCCGCCAC
CAACACCTCCGCCACCaccggtGGCGAAAATCTGTATTTTCAGGGAgCCGCGGccTCTAATTCCGCTGA
CGGTGACGGTTCAAATGCTACAGGGAGtTCTGCTGGTGCTGGCTCTGGAACGAGTGGCGGGGACAA
CACGAGTGATGGCTCCGGGGCGAGTGCCGGTGCAGCCAGCACAAATTCAAATGGGAACACGGGTA
GTGCGACTTCTGGGGGGGCCACAGGTAGCGATACGTCAGGAGCGACGGCTGGTAGTGGGGCTTCC
GACGGCGGAAACGGCGCAACAGCGTCATCAACTACAGGCAACGGAAATTCAAGCGGTACAACCG
CGACGACCGGAGGCGGTGATGCAGGGggGTCGACtAATGCTGTGGGCCAGGACACGCAGGAGGTC
ATCGTGGTGCCACACTCCTTGCCCTTTAAGGTGGTGGTGATCTCAGCCATCCTGGCCCTGGTGGTG
CTCACCATCATCTCCCTTATCATCCTCATCATGCTTTGGCAGAAGAAGCCACGTTAG2826GCGCGCA
ATAATG²⁸³⁹gaagacctattctctagaaagtataggaacttc²⁸⁷³GTAAGccggctacttgctttaaaaaacctcccacacctccccctgaacctg
aaacataaaatgaatgcaattgttgttgtt²⁹⁵⁴aacttgtttattgcagcttataatggttacaaataaagcaatagcatcacaaatttcacaaa
taaagcattatttcactgcattctagttgtggtttgtccaaactcatcaatgtatctta³⁰⁷⁶ACGCGTttcgaaTTAATTAACTCTGG³¹⁰²ATTT
CACGAAATATTTGAAAACATCACTACACTAGTAGAGGAATGAAGTCAGTGGACTTTCTTGTATATTTGTGTGTGCAGATGT
ACATAAAGATGAGTTGTTAACTTAGGATCTTTTCTTTTTATACAAGGAAAGCTTCCTAAGAATGTCT
AGGAAGAAGAGGAAGAATGACCCTTTGCATGGCACAGGGTTCTGCCCCTATTCTGAATATGTCATT
CCATCAAGGAGATCAAAAGCCTTTTTTTCTCCCCAGTATTTGGAAATTACTTTCTTGATGATGCTGC
CTTTTAAAAGCTTCACGTACATTATAGTTTTTTAAAAAAATCTTTGGACTGGATCTTACTGAAGTGC
AGTTGCTATATTAAAATTAGGGCATAGAGCACAGAAAAATCAAGACCATGAGAAGACATTTTACC
ATTTAGCTACTTTTTATAACTAAATACTCTTTAAATATTTTTATTTCAATACTGTGGATGGAAATGA
GAAGCATTCTAAATTTGAGTTAATATATTTTTATGAAGATATTTGAGAAAAGAAAAAAATAGCTTG
TATTCAGGTTCATTGGCTTTTGCTGGATGATCCACCTAAAGAAGTTACCTAATTTGGCCTTTTA^{3716A}
GTTGCTACTGAATCGCCTCTGG

### Sequence Annotation

| No. | Component sequences for Noc3L_GFP_SurfaceDisplay(Hivp24) | Location (Residues) |
|---|---|---|
| 1. | sgRNA target sequence | 1-23 |
| 2. | Left homology arm (LHA) + sgRNA without PAM + reoptimized ORF | 24-698 |
| 3. | Glycine linker | 708-821 |
| 4. | Gfp | 873-1601 |
| 5. | 1^{st} FRT sequence for FLP-FRT recombination | 1612-1645 |
| 6. | T2A peptide | 1656-1709 |
| 7. | Surface display sequence (epitope: BtuF) | 1710-2825 |
| 8. | 2^{nd} FRT sequence for FLP-FRT recombination | 2839-2872 |
| 7. | SV40 polyA signal | 2954-3075 |
| 8. | Right homology arm (RHA) | 3102-3715 |
| 9. | sgRNA target sequence | 3716-3738 |

### 7. Wdr12_mCherry_SurfaceDisplay(10× HA)

(SEQ ID NO: 18)
¹ACCTACCACTTCCCATGTTGGGG²⁴CCTCCAAAAACTCACTACTTAAGACTAATTGGATCAAAGTGT
TTACCAGTTGGAAAAATCTTGCATAAGTCTGCATTATAAAATGTGTTTAAAGAATTACAATTTAAT
TATTTTTATGTATATACGTAAGCTCTTACTGCCTAAGAATTCTTTCCAAATATAAGGCCTAGGGCTA
CTTGAATAATTTGTAATATACAATTAATGTGTTGTCCTTTAAAAATTTTTAATTTTCTTTAATAGGT
AAAACTGTATCCCTTTCAAACTTATGTATCTTGGCAGATGCTTTATAGAAAGTGCAACAGCATATT
ATGTCTCAACCAAATTTAAATGATAGCTTTTAATGTTTTAATAAACTGTATCATAGTATAGTAGTGA
AACAACGTTGGTCCCTTTACTCACTCTCAATGCAAGTTAACTGCTCACCCATAATTCCTTTTGTAAT
GAAAATCATTAGTATTTAATTAGGTTTAGCTATGATGTGAAATAATTATATTTATTTATGTTTTCTT
GTCTTTTTCTCTCCTTTTACACAGCTACTTCTGAGTGGAGGAGCAGACAATAAATTGTATTCCTACA
GATATTCACCTACCACTTCCCATGTTGGTGCA⁶³²gcggccgcc⁶⁴¹GGAGGtACTGGATCAGGTGGATCAG
CAGGAGGCGGTACTGGAGGTTCTGCTGGCGGtTCAGCTGGtGCGGGCGCGACGGGTGGAAGTACAG
CCGGAGGTGCCACGACAGCGTCC⁷⁵⁵CATCACCACCATCACCATCATCATCATCATTATCCATATGAC
GTACCTGATTATGCGgcgatcgctGGCGAGAACCTGTATTTTCAAGGGagctcgagtCCTTCAAGACTtGAGG
AAGAATTGAGACGGAGACTTACCGAGCCCGGCgcacagagtggtTTGGAGGTGCTTTTCCAGGGACCAG
GTgCTAGCGGAAGCGGAATGGTCAGTAAGGGTGAGGAGGACAACATGGCTATAATCAAAGAGTTT
ATGCGGTTTAAGGTCCATATGGAAGGTTCAGTTAATGGACATGAGTTCGAGATAGAAGGTGAGGG
TGAGGGGCGACCGTACGAAGGCACACAAACCGCAAAGTTGAAAGTCACCAAAGGTGGACCCTTGC
CCTTTGCTTGGGATATTCTCTCCCCTCAATTCATGTACGGCAGTAAGGCATACGTCAAACATCCCGC
TGACATCCCCGACTATCTGAAGCTGTCTTTCCCTGAGGGTTTTAAATGGGAGCGAGTGATGAACTT
CGAGGACGGGGGAGTGGTAACAGTGACTCAAGATTCCTCTTTGCAGGACGGGGAGTTCATATATA
AAGTGAAACTGCGGGGTACGAACTTTCCAAGTGACGGtCCCGTAATGCAGAAGAAGACGATGGGA
TGGGAGGCAAGCAGCGAGCGAATGTATCCTGAGGATGGAGCCCTTAAGGGAGAAATTAAGCAAC
GGCTGAAGTTGAAAGATGGTGGACATTATGATGCTGAGGTTAAAACAACTTATAAAGCCAAGAAA
CCAGTTCAGTTGCCAGGGGCGTATAACGTCAACATTAAACTGGACATTACATCTCACAATGAAGAT
TACACAATCGTTGAGCAATATGAaCGCGCGGAGGGTCGGCACTCAACGGGTGGCATGGACGAGTT
GTATAAA¹⁶⁶⁴GGCGCGCCC¹⁶⁷³ATAACTTCGTATAGCATACATTATACGAAGTTAT¹⁷⁰⁷CTGGGTCTGG
C¹⁷¹⁸GAAGGCAGAGGCTCCCTTTTGACATGcGGAGACGTCGAGGAGAACCCGGGTCCC¹⁷⁷²ATGGAG
ACAGACACACTCCTGCTATGGGTACTGCTcCTCTGGGTtCCAGGTTCCACTGGcGACggcggaccgTCTA
ACACAGCAAATGGGACTAGCACCACGAACGCATATCCTTACGAcGTtCCtGATTACGCTTCATCTGG
TGGAAGTGGcACCGGAGGGACTTATCCGTACGACGTaCCtGACTATGCTTCCACAAGCGGGGGGACt
GGTGGTGGCAGTTAtCCCTACGACGTTCCCGATTATGCGGGCACAGGTTCCGGGAGTACTGGTGGC
TCCTATCCtTATGATGTCCCCGATTAtGCGTCCAGCGGCGGCGGCTCTACTACAGGGGGtTATCCCTA
TGATGTTCCAGATTACGCCACTTCAGGTTCCGGGACTGGATCTGGAGGATAcCCTTAtGATGTACCA
GATTACGCTACTAGTGGCTCTGGCACAGGAGGCGGTTCATACCCCTACGATGTTCCGGACTACGCG
GGATCTGGGAGCGGCAGCACGACCAGTGGtTATCCCTATGACGTTCCAGACTACGCCGGGACGGGA
ACAGGGAGTTCCTCCGGCGGGTATCCATATGACGTACCAGATTATGCGACCTCTAGCGGAACCGG
GGGTTCTGGAGGGTATCCGTATGACGTGCCtGACTACGCCAATACTACATCTAACACTAGTGCATC
CGCGAATAGTaccggtGGCGAAAATCTGTATTTTCAGGGAgCCGCGGccTCTAATTCCGCTGACGGTGA
CGGTTCAAATGCTACAGGGAGtTCTGCTGGTGCTGGCTCTGGAACGAGTGGCGGGGACAACACGAG
TGATGGCTCCGGGGCGAGTGCCGGTGCAGCCAGCACAAATTCAAATGGGAACACGGGTAGTGCGA
CTTCTGGGGGGGCCACAGGTAGCGATACGTCAGGAGCGACGGCTGGTAGTGGGGCTTCCGACGGC
GGAAACGGCGCAACAGCGTCATCAACTACAGGCAACGGAAATTCAAGCGGTACAACCGCGACGA
CCGGAGGCGGTGATGCAGGGggGTCGACtAATGCTGTGGGCCAGGACACGCAGGAGGTCATCGTGG
TGCCACACTCCTTGCCCTTTAAGGTGGTGGTGATCTCAGCCATCCTGGCCCTGGTGGTGCTCACCAT
CATCTCCCTTATCATCCTCATCATGCTTTGGCAGAAGAAGCCACGTTAG²⁹⁵⁷gcgcgcaataat²⁹⁶⁹ataacttcgta
tagcatacattatacgaagttat³⁰⁰³aagccggctacttgctttaaaaaacctcccacacctccccctgaacctgaaacataaaatgaatgcaa
ttgttgttgtt³⁰⁸²aacttgtttattgcagcttataatggttacaaataaagcaatagcatcacaaatttcacaaataaagcatttattcactg
cattctagttgtggtttgtccaaactcatcaatgtatctta³²⁰⁴ACGCGTttcgaaTTAATTAA³²²⁴TGAAAGTGAACAATAATTTGACTATAG
AGATTATTTCTGTAAATGAAATTGGTAGAGAACCATGAAATTACATAGATGCAGATGCAGAAAGCAGCCTTTTGAAGTTT
ATATAATGTTTTCACCCTTCATAACAGCTAACGTATCACTTTTTCTTATTTTGTATTTATAATAAGAT
AGGTTGTGTTTATAAAATACAAACTGTGGCATACATTCTCTATACAAACTTGAAATTAAACTGAGT
TTTACATTTCTCTTTAAAGGTATTGGTTTGAATTCAGATTTGCTTTTTTATTTTTATTTGTTTTTTTTT
TTTTTGAGATGGAGTCTTGCTCTGTTGCCTAGGCTGGAGTGCAGTGGCGCAATCTCAACTCACTGC
AACCTCCGCTTCCTAGGTTCAATCGATTCTCCTGTCTCAACCTCCCAAGTAGCTGGGATTACAGGC
ACACATCACGATGTCCTGCTAATTTTTGTATTTTTAGTAGAGACGGGGTTTTGCCATGTTGGCCAGG
CTGGTCTTGAACTCCTGACCTCAGGTGATCTGCCCACCTCAGCCTCCCAAAGTGAGCCACTGTGCC
TGGCCGAATTAAGATTTGTTTTT³⁸²²ACCTACCACTTCCCATGTTGGGG

### Sequence Annotation

| No. | Component sequences for Wdr12_mCherry_SurfaceDisplay(10X HA) | Location (Residues) |
|---|---|---|
| 1. | sgRNA target sequence | 1-23 |
| 2. | Left homology arm (LHA) + sgRNA without PAM + reoptimized ORF | 24-631 |
| 3. | Glycine linker | 641-754 |
| 4. | HIS10-1XHA-Alfa-mCherry | 755-1663 |
| 5. | 1^{st} loxp sequence for Cre-lox recombination | 1673-1706 |
| 6. | T2A peptide | 1718-1771 |
| 7. | Surface display sequence (epitope: 10X HA) | 1772-2956 |
| 8. | 2nd loxp sequence for Cre-lox recombination | 2969-3002 |
| 7. | SV40 polyA signal | 3082-3203 |
| 8. | Right homology arm (RHA) | 3224-3821 |
| 9. | sgRNA target sequence | 3822-3844 |

### 8. Wdr12_mCherry_SurfaceDisplay(10× FLAG)

(SEQ ID NO: 19)
¹ACCTACCACTTCCCATGTTGGGG²⁴CCTCCAAAAACTCACTACTTAAGACTAATTGGATCAAAGTGT
TTACCAGTTGGAAAAATCTTGCATAAGTCTGCATTATAAAATGTGTTTAAAGAATTACAATTTAAT
TATTTTTATGTATATACGTAAGCTCTTACTGCCTAAGAATTCTTTCCAAATATAAGGCCTAGGGCTA
CTTGAATAATTTGTAATATACAATTAATGTGTTGTCCTTTAAAAATTTTTAATTTTCTTTAATAGGT
AAAACTGTATCCCTTTCAAACTTATGTATCTTGGCAGATGCTTTATAGAAAGTGCAACAGCATATT
ATGTCTCAACCAAATTTAAATGATAGCTTTTAATGTTTTAATAAACTGTATCATAGTATAGTAGTGA
AACAACGTTGGTCCCTTTACTCACTCTCAATGCAAGTTAACTGCTCACCCATAATTCCTTTTGTAAT
GAAAATCATTAGTATTTAATTAGGTTTAGCTATGATGTGAAATAATTATATTTATTTATGTTTTCTT
GTCTTTTTCTCTCCTTTTACACAGCTACTTCTGAGTGGAGGAGCAGACAATAAATTGTATTCCTACA
GATATTCACCTACCACTTCCCATGTTGGTGCA⁶³²gcggccgcc⁶⁴¹GGAGGtACTGGATCAGGTGGATCAG
CAGGAGGCGGTACTGGAGGTTCTGCTGGCGGtTCAGCTGGtGCGGGCGCGACGGGTGGAAGTACAG
CCGGAGGTGCCACGACAGCGTCC⁷⁵⁵CATCACCACCATCACCATCATCATCATCATTATCCATATGAC
GTACCTGATTATGCGgcgatcgctGGCGAGAACCTGTATTTTCAAGGGagctcgagtCCTTCAAGACTtGAGG
AAGAATTGAGACGGAGACTTACCGAGCCCGGCgcacagagtggtTTGGAGGTGCTTTTCCAGGGACCAG
GTgCTAGCGGAAGCGGAATGGTCAGTAAGGGTGAGGAGGACAACATGGCTATAATCAAAGAGTTT
ATGCGGTTTAAGGTCCATATGGAAGGTTCAGTTAATGGACATGAGTTCGAGATAGAAGGTGAGGG
TGAGGGGCGACCGTACGAAGGCACACAAACCGCAAAGTTGAAAGTCACCAAAGGTGGACCCTTGC
CCTTTGCTTGGGATATTCTCTCCCCTCAATTCATGTACGGCAGTAAGGCATACGTCAAACATCCCGC
TGACATCCCCGACTATCTGAAGCTGTCTTTCCCTGAGGGTTTTAAATGGGAGCGAGTGATGAACTT
CGAGGACGGGGGAGTGGTAACAGTGACTCAAGATTCCTCTTTGCAGGACGGGGAGTTCATATATA
AAGTGAAACTGCGGGGTACGAACTTTCCAAGTGACGGtCCCGTAATGCAGAAGAAGACGATGGGA
TGGGAGGCAAGCAGCGAGCGAATGTATCCTGAGGATGGAGCCCTTAAGGGAGAAATTAAGCAAC
GGCTGAAGTTGAAAGATGGTGGACATTATGATGCTGAGGTTAAAACAACTTATAAAGCCAAGAAA
CCAGTTCAGTTGCCAGGGGCGTATAACGTCAACATTAAACTGGACATTACATCTCACAATGAAGAT
TACACAATCGTTGAGCAATATGAaCGCGCGGAGGGTCGGCACTCAACGGGTGGCATGGACGAGTT
GTATAAA¹⁶⁶⁴GGCGCGCCC¹⁶⁷³ATAACTTCGTATAGCATACATTATACGAAGTTAT¹⁷⁰⁷CTGGGTCTGG
C¹⁷¹⁸GAAGGCAGAGGCTCCCTTTTGACATGcGGAGACGTCGAGGAGAACCCGGGTCCC¹⁷⁷²ATGGAG
ACAGACACACTCCTGCTATGGGTACTGCTcCTCTGGGTtCCAGGTTCCACTGGcGACggcggaccgTCTA
ACACAGCAAATGGGACTAGCACCACGAACGCAGACTACAAGGACGACGACGATAAGACCGGCAG
CGATTATAAGGATGATGACGATAAGAGTTCCGGCGACTATAAGGACGACGATGATAAGGGGACCA
CTGAtTACAAAGACGATGACGACAAAGGCGGGTCCGACTATAAGGATGACGATGACAAGAGCGGA
AGTGATTAcAAAGATGATGACGACAAGACCGGGACTGATTATAAAGATGATGATGATAAAGGCTC
CAGTGATTAtAAAGAcGACGACGACAAGGGCAGTGGAGACTAcAAAGACGACGAtGACAAGGGTAC
TGGCGATTACAAGGATGATGATGACAAGAATACTACATCTAACACTAGTGCATCCGCGAATAGTac
cggtGGCGAAAATCTGTATTTTCAGGGAgCCGCGGccTCTAATTCCGCTGACGGTGACGGTTCAAATG
CTACAGGGAGtTCTGCTGGTGCTGGCTCTGGAACGAGTGGCGGGGACAACACGAGTGATGGCTCCG
GGGCGAGTGCCGGTGCAGCCAGCACAAATTCAAATGGGAACACGGGTAGTGCGACTTCTGGGGGG
GCCACAGGTAGCGATACGTCAGGAGCGACGGCTGGTAGTGGGGCTTCCGACGGCGGAAACGGCGC
AACAGCGTCATCAACTACAGGCAACGGAAATTCAAGCGGTACAACCGCGACGACCGGAGGCGGT
GATGCAGGGggGTCGACtAATGCTGTGGGCCAGGACACGCAGGAGGTCATCGTGGTGCCACACTCC
TTGCCCTTTAAGGTGGTGGTGATCTCAGCCATCCTGGCCCTGGTGGTGCTCACCATCATCTCCCTTA
TCATCCTCATCATGCTTTGGCAGAAGAAGCCACGTTAG²⁷³⁸gcgcgcaataat²⁷⁵⁰ataacttcgtatagcatacattatacgaa
gttat²⁷⁸⁴aagccggctacttgctttaaaaaacctcccacacctccccctgaacctgaaacataaaatgaatgcaattgttgttgtt²⁸⁶³aact
tgtttattgcagcttataatggttacaaataaagcaatagcatcacaaatttcacaaataaagcatttttttcactgcattctagttgtggtt
tgtccaaactcatcaatgtatctta²⁹⁸⁵ACGCGTttcgaaTTAATTAA³⁰⁰⁵TGAAAGTGAACAATAATTTGACTATAGAGATTATTTCTGTAA
ATGAAATTGGTAGAGAACCATGAAATTACATAGATGCAGATGCAGAAAGCAGCCTTTTGAAGTTTATATAATGTTTT
CACCCTTCATAACAGCTAACGTATCACTTTTTCTTATTTTGTATTTATAATAAGATAGGTTGTGTTT
ATAAAATACAAACTGTGGCATACATTCTCTATACAAACTTGAAATTAAACTGAGTTTTACATTTCT
CTTTAAAGGTATTGGTTTGAATTCAGATTTGCTTTTTTATTTTTATTTGTTTTTTTTTTTTTTGAGATG
GAGTCTTGCTCTGTTGCCTAGGCTGGAGTGCAGTGGCGCAATCTCAACTCACTGCAACCTCCGCTT
CCTAGGTTCAATCGATTCTCCTGTCTCAACCTCCCAAGTAGCTGGGATTACAGGCACACATCACGA
TGTCCTGCTAATTTTTGTATTTTTAGTAGAGACGGGGTTTTGCCATGTTGGCCAGGCTGGTCTTGAA
CTCCTGACCTCAGGTGATCTGCCCACCTCAGCCTCCCAAAGTGAGCCACTGTGCCTGGCCGAATTA
AGATTTGTTTTT³⁶⁰³ACCTACCACTTCCCATGTTGGGG

### Sequence Annotation

| No. | Component sequences for Wdr12_mCherry_SurfaceDisplay(10X FLAG) | Location (Residues) |
|---|---|---|
| 1. | sgRNA target sequence | 1-23 |
| 2. | Left homology arm (LHA) + sgRNA without PAM + reoptimized ORF | 24-631 |
| 3. | Glycine linker | 641-754 |
| 4. | HIS10-1XHA-Alfa-mCherry | 755-1663 |
| 5. | 1^{st} loxp sequence for Cre-lox recombination | 1673-1706 |
| 6. | T2A peptide | 1718-1771 |
| 7. | Surface display sequence (epitope: 10X FLAG) | 1772-2737 |
| 8. | 2nd loxp sequence for Cre-lox recombination | 2750-2783 |
| 7. | SV40 polyA signal | 2863-2984 |
| 8. | Right homology arm (RHA) | 3005-3602 |
| 9. | sgRNA target sequence | 3603-3625 |

### SgRNA Sequences

| | Gene | |
|---|---|---|
| No. | target | sgRNA sequence |
| 1. | Rrp12 | GUCCCACCUGGGAACCUCGC (SEQ ID NO: 20) |
| 2. | Pes1 | AGACCUCACCGCCUCAUCGU (SEQ ID NO: 21) |
| 3. | Noc3L | AGUUGCUACUGAAUCGCCUC (SEQ ID NO: 22) |
| 4. | Wdr12 | ACCUACCACUUCCCAUGUUG (SEQ ID NO: 23) |

Sequence of Flp Recombinase-P2a-Mbfp used for Epitope Recycling and FACS Selection

(SEQ ID NO: 24)
¹ATGCCACAATTTGATATATTATGTAAAACACCACCTAAGGTGCTTGTTCGTCAGTTTGTGGAAAGG
TTTGAAAGACCTTCAGGTGAGAAAATAGCATTATGTGCTGCTGAACTAACCTATTTATGTTGGATG
ATTACACATAACGGAACAGCAATCAAGAGAGCCACATTCATGAGCTATAATACTATCATAAGCAA
TTCGCTGAGTTTGGATATTGTCAACAAGTCACTGCAGTTTAAATACAAGACGCAAAAAGCAACAAT
TCTGGAAGCCTCATTAAAGAAATTGATaCCTGCTTGGGAATTTACAATTATTCCTTACTATGGACAA
AAACATCAATCTGATATCACTGATATTGTAAGTAGTTTGCAATTACAGTTCGAATCATCGGAAGAA
GCAGATAAGGGAAATAGCCACAGTAAAAAAATGCTTAAAGCACTTCTAAGTGAGGGTGAAAGCAT
CTGGGAGATCACTGAGAAAATACTAAATTCGTTTGAGTATACTTCGAGATTTACAAAAACAAAAA
CTTTATACCAATTCCTCTTCCTAGCTACTTTCATCAATTGTGGAAGATTCAGCGATATTAAGAACGT
TGATCCGAAATCATTTAAATTAGTCCAAAATAAGTATCTGGGAGTAATAATCCAGTGTTTAGTGAC
AGAGACAAAGACAAGCGTTAGTAGGCACATATACTTCTTTAGCGCAAGGGGTAGGATCGATCCAC
TTGTATATTTGGATGAATTTTTGAGGAATTCTGAACCAGTCCTAAAACGAGTAAATAGGACCGGCA
ATTCTTCAAGCAACAAaCAGGAATACCAATTATTAAAAGATAACTTAGTCAGATCGTACAACAAAG
CTTTGAAGAAAAATGCGCCTTATTCAATCTTTGCTATAAAAAATGGCCCAAAATCTCACATTGGAA
GACATTTGATGACCTCATTTCTTTCAATGAAGGGCCTAACGGAGTTGACTAATGTTGTGGGAAATT
GGAGCGATAAGCGTGCTTCTGCCGTGGCCAGGACAACGTATACTCATCAGATAACAGCAATACCT
GATCACTACTTCGCtCTAGTTTCTCGGTACTATGCtTATGATCCAATATCAAAGGAAATGATAGCATT
GAAGGATGAGACTAATCCAATTGAGGAGTGGCAGCATATAGAACAGCTAAAGGGTAGTGCTGAA
GGAAGCATACGATACCCCGCATGGAATGGGATAATATCACAGGAGGTACTAGACTACCTTTCATC
CTACATAAATAGACGCATA¹²⁷⁰gcggccgccGGAAGCGGA¹²⁸⁸gccactaacttctccctgttgaaacaagcaggggatgtcgaaga
gaatcccgggcca¹³⁴⁵ACCGGTGGCGCGCCTGGT¹³⁶³atgagcgagctgattaaggagaacatgcacatgaagctgtacatggagggcacc
gtggacaaccatcacttcaagtgcacatccgagggcgaaggcaagccttacgagggcacccagaccatgagaatcaaggtggtcgagggc
ggccctctccccttcgccttcgacatcctggctactagcttcctctacggcagcaagaccttcatcaaccacacccagggcatccccgac
ttcttcaagcagtccttccctgagggcttcacatgggagagagtcaccacatacgaagacgggggcgtgctgaccgctacccaggacacc
agcctccaggacggctgcctcatctacaacgtcaagatcagaggggtgaacttcacatccaacggccctgtgatgcagaagaaaacactc
ggctgggaggccttcaccgagacgctgtaccccgctgacggcggcctggaaggcagaaacgacatggccctgaagctcgtgggcgggagc
catctgatcgcaaacgccaagaccacatatagatccaagaaacccgctaagaacctcaagatgcctggcgtctactatgtggactacaga
ctggaaagaatcaaggaggccaacaacgagacctacgtcgagcagcacgaggtggcagtggccagatactgcgacctgcctagcaaact
ggggcacaaacttaattaa

### Sequence Annotation

| | Component sequences | Location |
|---|---|---|
| No. | for Flp_2a_Bfp | (Residues) |
| 1. | Flp recombinase | 1-1269 |
| 2. | P2A sequence | 1288-1344 |
| 3. | Monomeric blue | 1363-2064 |
| | fluorescent protein | |
| | (mBFP) | |

The foregoing examples are meant to illustrate but not limit the disclosure.

1. A method for producing a population of eukaryotic cells comprising a homozygous insertion of first and second DNA segments into a chromosomal locus, the method comprising introducing into the cells:
a) a first and second double stranded (ds) DNA repair template, each of which is optionally provided as a component of a plasmid:
the first dsDNA repair template comprising:
i) a 5′ homology segment comprising a dsDNA sequence for integration into a chromosome sequence that is homologous to the 5′ homology segment;
ii) a 3′ homology segment comprising a dsDNA sequence for integration into a chromosome sequence that is homologous to the 3′ homology segment;
iii) a sequence comprising a modified open reading frame (“ORF”), the modified ORF comprising at least a single nucleotide difference relative to the endogenous ORF in the chromosome;
iv) sequentially in a 5′>3′ direction:
a sequence encoding a ribosomal peptide skipping domain, a sequence encoding a secretion signal; a sequence encoding a first epitope that can be recognized with specificity by a detectably labeled first antibody, optionally a sequence encoding a linker, and a sequence encoding a transmembrane domain (TMD);
b) a second dsDNA repair template comprising i)-iv) of a), with the exception that the second dsDNA repair template comprises in iv) a sequence encoding a second epitope that can be recognized with specificity by a detectably labeled second antibody;
c) a Cas enzyme or DNA sequence encoding the Cas enzyme;
d) a guide RNA or a DNA sequence encoding the guide RNA, wherein the guide RNA comprises a sequence that recognizes a protospacer in the chromosome such that a complex comprising the Cas enzyme and the guide RNA can facilitate homologous recombination of the first and second dsDNA repair templates into a first and second allele of the same chromosomal locus, thereby providing a eukaryotic cell comprising a homozygous replacement of the first and second alleles with the first and second dsDNA repair templates, and expression of the first allele comprises expression of the first epitope, and expression of the second allele comprises expression of the second epitope.

2. The method of claim 1, wherein the sequences encoding the first and second epitopes are repeated in the first and second dsDNA repair templates at least two times.

3. The method of claim 1, wherein the modified ORF comprises a sequence encoding a corrected version of an ORF that contains one or more deleterious mutations, a protein that produces a fluorescent signal, or a sequence used for purification of the protein.

4. The method of claim 1, wherein the first and second dsDNA repair templates comprise sequences encoding recombinase recognition sequences, wherein the recombinase recognition sequences flank at least the sequences encoding the first and second epitope of iv), said recombinase recognition sequences being operative with a recombinase that can excise chromosomal segments comprising the sequences that encode at least the first and second epitopes.

5. The method of claim 4, further comprising expressing a recombinase that recognizes the recombinase recognition sequences in the cells, such that the recombinase excises the sequence of iv) encoding at least the first and second epitopes, thereby removing the sequences encoding the first and second epitopes and leaving the sequence encoding the modified ORF in the first and second alleles.

6. A method for producing a population of single cell clones comprising a homozygous chromosomal insertion, the method comprising providing a population of cells made according to claim 1, and separating cells from the population that express the first and second epitopes from cells that do not express the first and second epitopes using the detectably labeled antibodies that bind with specificity to the first and second epitopes.

7. The method of claim 6, wherein the sorting comprises fluorescence activated cell sorting (FACS).

8. The method of claim 6, wherein a time period from which the first and second dsDNA repair templates, the Cas enzyme, and the guide RNA are introduced into the cells and are separated from the cells that do not express the first and second epitopes is less than a reference value.

9. The method of claim 8, wherein the time period is 1-120 days.

10. The method of claim 6, wherein at least 10% of the cells separated from the population into which the first and second dsDNA repair templates, the Cas enzyme, and the guide RNA are introduced comprise the homozygous chromosomal insertion.

11. The method of claim 10, wherein at least 35% of the cells separated from the population into which the first and second dsDNA repair templates, the Cas enzyme, and the guide RNA are introduced comprise the homozygous chromosomal insertion.

12. The method of claim 11, further comprising expressing a recombinase that recognizes the recombinase recognition sequences in the cells, such that the recombinase excises the sequence of iv) encoding at least the first and second epitopes, thereby leaving the sequence encoding the modified ORF in the first and second alleles

13. A single cell or population of cells made according to the method of claim 1.

14. The single cell or population of cells of claim 13, wherein the sequence of iv) is removed by operation of the recombinase.

15. A kit comprising one or more DNA vectors for making the cells of claim 1.

16. The kit of claim 15, wherein the vector(s) comprise one or more cloning sites for introducing into the vector the 5′ homology segment and the 3′ homology segment;
ii) a sequence encoding a ribosomal skipping peptide;
iii) sequentially in a 5′>3′ direction:
a sequence encoding a secretion signal; a sequence encoding a first epitope that can be recognized with specificity by a detectably labeled first antibody, optionally a sequence encoding a linker, and a sequence encoding a transmembrane domain (TMD); and
a sequence encoding a secretion signal; a sequence encoding a second epitope that can be recognized with specificity by a detectably labeled first antibody, optionally a sequence encoding a linker, and a sequence encoding a transmembrane domain (TMD);
the kit optionally further comprising distinctly labeled first and second antibodies that separately recognize with specificity the first and second epitopes.

17. The kit of claim 16, the vector(s) further comprising sequences encoding recombinase recognition sequences, wherein the recombinase recognition sequences flank at least the sequences encoding the first and second epitopes.

18. The kit of claim 17, further comprising a recombinase that recognizes the first and second recombination recognition sequences.


SEQUENCE LISTING (24 sequences)

SEQ ID NO: 1 - protein, 4 aa, artificial sequence
  llinker
SGSG

SEQ ID NO: 2 - protein, 6 aa, artificial sequence
  linker
GASGSG

SEQ ID NO: 3 - protein, 38 aa, artificial sequence
  linker
GGTGSGGSAGGTGGSAGGSAGAGGATGGSTAGGATTAS

SEQ ID NO: 4 - protein, 100 aa, artificial sequence
  linker
SNSADGDGSNATGSSAGAGSGTSGGDNTSDGSGASAGAASTNSNGNTGSATSGGATGSDTSGATAGSGASDGGNGATASSTTGNGNSSGTTATTGGGDAG

SEQ ID NO: 5 - protein, 18 aa, artificial sequence
  linker
EGRGSLLTCGDVEENPGP

SEQ ID NO: 6 - protein, 19 aa, artificial sequence
  ribosomal skipping sequence
ATNFSLLKQAGDVEENPGP

SEQ ID NO: 7 - protein, 20 aa, artificial sequence
  ribosomal skipping sequence
QCTNYALLKLAGDVESNPGP

SEQ ID NO: 8 - protein, 22 aa, artificial sequence
  ribosomal skipping sequence
VKQTLNFDLLKLAGDVESNPGP

SEQ ID NO: 9 - protein, 20 aa, Homo sapiens
METDTLLLWVLLLWVPGSTG

SEQ ID NO: 10 - protein, 26 aa, Homo sapiens
MATGSRTSLLLAFGLLCLPWLQEGSA

SEQ ID NO: 11 - protein, 21 aa, Mus musculus
METDTLLLWVLLLWVPGSTGD

SEQ ID NO: 12 - DNA, 3561 bp, artificial sequence
  insertion template
ccggcgaggttcccaggtgggaccccaggatggtcttgatcccctgaccttgtgatctgcccacctcggcctcccaaagtgctgggattacaggcatgagccaccacgcccagccatagtcatcatttttaatagctttgtataatttgcttttctaatccctttattggtaggaaattagagttgtttccgactttggcccttaaattgggttatgtgtaggactgctttggaaactaatgttactagggaaatggtgttgtaaagttctagcttctgcgggttgtaagttacctttcaatggagggatgggtgggcagagggagctttgaccttctctggacatacattagaggaaaaatggaagggaggcctgtttccagggggataattgtgccaaagtggaatgtccaggtcaggacatgagccgtgtggaagctggaaccacgtgaggtctgcctagttcatgtgctggccaccacctggaggcccccttctcatccctgctggcgctgggggtgagccatcatttggcaacaggagggggcctcctattctcagccagatgtgacccttccgttccttggccctgcaggaagaagatgaagctgcagggacagttcaaaggcctggtgaaggctgctcggcgaggttcccaggtgggacacaaaaatcgccggaaagatagaagacccgcggccgccgggggcacgggaagtggtggatcagccggtggcactggtggctctgccggagggtcagcgggagcagggggagccacaggcggatctacggctggaggggcgacaacggcctctgcgatcgctggcgaaaatctgtattttcagggaggagctagcggaagcggaatggtcagtaagggtgaggaggacaacatggctataatcaaagagtttatgcggtttaaggtccatatggaaggttcagttaatggacatgagttcgagatagaaggtgagggtgaggggcgaccgtacgaaggcacacaaaccgcaaagttgaaagtcaccaaaggtggacccttgccctttgcttgggatattctctcccctcaattcatgtacggcagtaaggcatacgtcaaacatcccgctgacatccccgactatctgaagctgtctttccctgagggttttaaatgggagcgagtgatgaacttcgaggacgggggagtggtaacagtgactcaagattcctctttgcaggacggggagttcatatataaagtgaaactgcggggtacgaactttccaagtgacggtcccgtaatgcagaagaagacgatgggatgggaggcaagcagcgagcgaatgtatcctgaggatggagcccttaagggagaaattaagcaacggctgaagttgaaagatggtggacattatgatgctgaggttaaaacaacttataaagccaagaaaccagttcagttgccaggggcgtataacgtcaacattaaactggacattacatctcacaatgaagattacacaatcgttgagcaatatgaacgcgcggagggtcggcactcaacgggtggcatggacgagttgtataaaggcgcgcccggaagcggagctactaacttcagcctgctgaagcaggctggagacgtggaggagaaccctggacctatgggctggtcatgtatcattctgtttctggtcgcaaccgcaactggagtgcattcacaggtgcagctcggcggaccgacgaatcctgaaaaggtgaaggtctggtacgagaggtcccttgttctgcaaaaggaggcagactcactttgtactttcatagatgatttgaagctggcgatagcacgagagagtgatggtaaagacgcgaaagtgaacgacatacgacgcaaagataaccttgacgcttcaagtgtcgtgatgctgaacccaatcaacggaaaaggctcaacccttcggaaggaagtggataagtttcgggagcttgtagctacgttgatgacggacaaggccaagctcaagttgattgaacaggcactgaatactgaaagcggaacgaagggtaagagctgggagtcctcactgttcgagaatatgccaacagttgccgcgattacgctcctgacgaagctccagtcagacgtacggtacgcgcaaggtgaggtacttgctgatcttgtaaaagggagcggaactaccggtttggaagtgcttttccaggggcctgccgcggcctctaattccgctgacggtgacggttcaaatgctacagggagttctgctggtgctggctctggaacgagtggcggggacaacacgagtgatggctccggggcgagtgccggtgcagccagcacaaattcaaatgggaacacgggtagtgcgacttctgggggggccacaggtagcgatacgtcaggagcgacggctggtagtggggcttccgacggcggaaacggcgcaacagcgtcatcaactacaggcaacggaaattcaagcggtacaaccgcgacgaccggaggcggtgatgcaggggggtcgactaatgctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgttaggcgcgcaataatgccggctacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgtttcgaattaattaaaggttcccaggtgggacacaaaaaccgcagaaaggatcgtcgaccctgaggcccagggcccctgggctgccctgtggtccagtctgaggccctttcagcccccaggctgccttgccaccagctccaggtgctcaagattctggcagagcctggactcaggatgacttggaactagggcttggctctcagaagtcctggattttggaaactccaaatggaatcacccttcagagacatccctggtgcctggagatgggaatgtggcctcagtgcctctgagtaggtgccatgaggcacctttgctttctgcccagagtggccatgagcaccagaacagatgatctccatttccgccagctgcctgtagccacgtggcatcctgcctgtggtctgggtgagatttactgtgaccagatgtagaataaatgtgtctcatcctgcattttttttctagaaactgtttcatagtctgccccctccaggggtaagaacagtgtgcagttgttggcagcagtggcctgacctcttcctgtctaactccttacatccagtccagggcatatcataaggctttgcccataggacaggctttggaacttgcccgggagcacccacctgtgccggcgaggttcccaggtgggac

SEQ ID NO: 13 - DNA, 3396 bp, artificial sequence
  insertion template
ccggcgaggttcccaggtgggaccccaggatggtcttgatcccctgaccttgtgatctgcccacctcggcctcccaaagtgctgggattacaggcatgagccaccacgcccagccatagtcatcatttttaatagctttgtataatttgcttttctaatccctttattggtaggaaattagagttgtttccgactttggcccttaaattgggttatgtgtaggactgctttggaaactaatgttactagggaaatggtgttgtaaagttctagcttctgcgggttgtaagttacctttcaatggagggatgggtgggcagagggagctttgaccttctctggacatacattagaggaaaaatggaagggaggcctgtttccagggggataattgtgccaaagtggaatgtccaggtcaggacatgagccgtgtggaagctggaaccacgtgaggtctgcctagttcatgtgctggccaccacctggaggcccccttctcatccctgctggcgctgggggtgagccatcatttggcaacaggagggggcctcctattctcagccagatgtgacccttccgttccttggccctgcaggaagaagatgaagctgcagggacagttcaaaggcctggtgaaggctgctcggcgaggttcccaggtgggacacaaaaatcgccggaaagatagaagacccgcggccgccgggggcacgggaagtggtggatcagccggtggcactggtggctctgccggagggtcagcgggagcagggggagccacaggcggatctacggctggaggggcgacaacggcctctgcgatcgctggcgaaaatctgtattttcagggaggagctagcggaagcggaatggtcagtaagggtgaggaggacaacatggctataatcaaagagtttatgcggtttaaggtccatatggaaggttcagttaatggacatgagttcgagatagaaggtgagggtgaggggcgaccgtacgaaggcacacaaaccgcaaagttgaaagtcaccaaaggtggacccttgccctttgcttgggatattctctcccctcaattcatgtacggcagtaaggcatacgtcaaacatcccgctgacatccccgactatctgaagctgtctttccctgagggttttaaatgggagcgagtgatgaacttcgaggacgggggagtggtaacagtgactcaagattcctctttgcaggacggggagttcatatataaagtgaaactgcggggtacgaactttccaagtgacggtcccgtaatgcagaagaagacgatgggatgggaggcaagcagcgagcgaatgtatcctgaggatggagcccttaagggagaaattaagcaacggctgaagttgaaagatggtggacattatgatgctgaggttaaaacaacttataaagccaagaaaccagttcagttgccaggggcgtataacgtcaacattaaactggacattacatctcacaatgaagattacacaatcgttgagcaatatgaacgcgcggagggtcggcactcaacgggtggcatggacgagttgtataaaggcgcgcccggaagcggagctactaacttcagcctgctgaagcaggctggagacgtggaggagaaccctggacctatgggctggtcatgtatcattctgtttctggtcgcaaccgcaactggagtgcattcacaggtgcagctcggcggaccgtcccaactgagccaagtaacgccagtggatgaagtggacggaaccagaacgtatcgcgttcgggggcaactctttttcgtctctacccatgacttcttgcaccagttcgactttacccatccagcaaggcgggtggtgattgacctctctgacgctcacttttgggatgggagtgccgtaggagctttggacaaggtgatgctgaagtttatgagacagggcacgagtgtcgagctgcgcgggctgaacgctgcaagtgccactcttgttgaacggcttgggagcggaactaccggtggcgaaaatctgtattttcagggagccgcggcctctaattccgctgacggtgacggttcaaatgctacagggagttctgctggtgctggctctggaacgagtggcggggacaacacgagtgatggctccggggcgagtgccggtgcagccagcacaaattcaaatgggaacacgggtagtgcgacttctgggggggccacaggtagcgatacgtcaggagcgacggctggtagtggggcttccgacggcggaaacggcgcaacagcgtcatcaactacaggcaacggaaattcaagcggtacaaccgcgacgaccggaggcggtgatgcaggggggtcgactaatgctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgttaggcgcgcaataatgccggctacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgtttcgaattaattaaaggttcccaggtgggacacaaaaaccgcagaaaggatcgtcgaccctgaggcccagggcccctgggctgccctgtggtccagtctgaggccctttcagcccccaggctgccttgccaccagctccaggtgctcaagattctggcagagcctggactcaggatgacttggaactagggcttggctctcagaagtcctggattttggaaactccaaatggaatcacccttcagagacatccctggtgcctggagatgggaatgtggcctcagtgcctctgagtaggtgccatgaggcacctttgctttctgcccagagtggccatgagcaccagaacagatgatctccatttccgccagctgcctgtagccacgtggcatcctgcctgtggtctgggtgagatttactgtgaccagatgtagaataaatgtgtctcatcctgcattttttttctagaaactgtttcatagtctgccccctccaggggtaagaacagtgtgcagttgttggcagcagtggcctgacctcttcctgtctaactccttacatccagtccagggcatatcataaggctttgcccataggacaggctttggaacttgcccgggagcacccacctgtgccggcgaggttcccaggtgggac

SEQ ID NO: 14 - DNA, 3571 bp, artificial sequence
  insertion template
cccacgatgaggcggtgaggtctgaccagcgttggcaacatattgagaccctgtctctaccccccaaaaaaaaaaagaaagggctacgcatggtggtgcacacctgtagtcaatcccagctactccggaggctgaagtgggaggatcgtttgaggctgcagtgagctatgattgtgccactgtgctccaggctgagcaacagagaaagaccctgtccctttaaaaaaattaaaaatatattgtcagatgaccccggaaagaaggttcttcctgttgtacccctttccaccagctcctggtgaaggttctagtggcatccagctttcccaggtggtgtagggaaatggggcagttgccaaggctccttccagctctgggagtttaggattctcttatctcgagatttgtgggcccatgaaataatgttgttaaagcagggctagcgcatgttttctcaccatgaagtgggtcaggtagatttttttcctgtgagaatttgtgaccttttcttgaagctctgcttttaagggatatagctttgagttctgtgccccccaccctcccttctacacatacctcagcctgaccttcgccttccccctcacaggccaacaagctggcggagaagcggaaagcacacgatgaggctgtaagatcagagaagaaggcgaaaaaggcgcgacctgaggcggccgccgggggcacgggaagtggtggatcagccggtggcactggtggctctgccggagggtcagcgggagcagggggagccacaggcggatctacggctggaggggcgacaacggcctctgcgatcgctggcgaaaatctgtattttcagggaggagctagcggaagcggaatggtcagtaagggtgaggaggacaacatggctataatcaaagagtttatgcggtttaaggtccatatggaaggttcagttaatggacatgagttcgagatagaaggtgagggtgaggggcgaccgtacgaaggcacacaaaccgcaaagttgaaagtcaccaaaggtggacccttgccctttgcttgggatattctctcccctcaattcatgtacggcagtaaggcatacgtcaaacatcccgctgacatccccgactatctgaagctgtctttccctgagggttttaaatgggagcgagtgatgaacttcgaggacgggggagtggtaacagtgactcaagattcctctttgcaggacggggagttcatatataaagtgaaactgcggggtacgaactttccaagtgacggtcccgtaatgcagaagaagacgatgggatgggaggcaagcagcgagcgaatgtatcctgaggatggagcccttaagggagaaattaagcaacggctgaagttgaaagatggtggacattatgatgctgaggttaaaacaacttataaagccaagaaaccagttcagttgccaggggcgtataacgtcaacattaaactggacattacatctcacaatgaagattacacaatcgttgagcaatatgaacgcgcggagggtcggcactcaacgggtggcatggacgagttgtataaaggcgcgcccggaagcggagctactaacttcagcctgctgaagcaggctggagacgtggaggagaaccctggacctatgggctggtcatgtatcattctgtttctggtcgcaaccgcaactggagtgcattcacaggtgcagctcggcggaccgacgaatcctgaaaaggtgaaggtctggtacgagaggtcccttgttctgcaaaaggaggcagactcactttgtactttcatagatgatttgaagctggcgatagcacgagagagtgatggtaaagacgcgaaagtgaacgacatacgacgcaaagataaccttgacgcttcaagtgtcgtgatgctgaacccaatcaacggaaaaggctcaacccttcggaaggaagtggataagtttcgggagcttgtagctacgttgatgacggacaaggccaagctcaagttgattgaacaggcactgaatactgaaagcggaacgaagggtaagagctgggagtcctcactgttcgagaatatgccaacagttgccgcgattacgctcctgacgaagctccagtcagacgtacggtacgcgcaaggtgaggtacttgctgatcttgtaaaagggagcggaactaccggtttggaagtgcttttccaggggcctgccgcggcctctaattccgctgacggtgacggttcaaatgctacagggagttctgctggtgctggctctggaacgagtggcggggacaacacgagtgatggctccggggcgagtgccggtgcagccagcacaaattcaaatgggaacacgggtagtgcgacttctgggggggccacaggtagcgatacgtcaggagcgacggctggtagtggggcttccgacggcggaaacggcgcaacagcgtcatcaactacaggcaacggaaattcaagcggtacaaccgcgacgaccggaggcggtgatgcaggggggtcgactaatgctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgttaggcgcgcaataatgccggctacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgtttcgaattaattaaatgaggcggtgaggtctgagaagaaggccaagaaggcaaggccggagtgagtgcctgcggcccctcacagggctgaggccagcccctagcagctggatgtggcagaggcaggccagaggacctaagtgtgatggaccagagtcacttctcctcctcctttctccagccagccctgacccctcatgctctctggctgggccagtgggcagccctcgcttcccttggatggagctgccctgctggtgcctggtcagagaagaggcctctgtgcccagcctgattctctgctcccaggagccagtgacatgaggtgcagaggcccacccagccccctacctactgcccccattcatcctggctttccacagccccctcccacacagttggacccgtgattctcagggtgctgtgatggggtgagggtagggggagcatttgttattaaatgactggacttttgtgccaattgcattttgtgtccatgagccttcctagggttggaggaggcctacctagcactctatgctgcaggctgggccagccctgggtatttactgagacagagctgggcactgctcagagctctctggatgtccaaggacccctccaggtccagggatgccaaaaggtaggtgcacccacgatgaggcggtgaggtct

SEQ ID NO: 15 - DNA, 3406 bp, artificial sequence
  insertion template
cccacgatgaggcggtgaggtctgaccagcgttggcaacatattgagaccctgtctctaccccccaaaaaaaaaaagaaagggctacgcatggtggtgcacacctgtagtcaatcccagctactccggaggctgaagtgggaggatcgtttgaggctgcagtgagctatgattgtgccactgtgctccaggctgagcaacagagaaagaccctgtccctttaaaaaaattaaaaatatattgtcagatgaccccggaaagaaggttcttcctgttgtacccctttccaccagctcctggtgaaggttctagtggcatccagctttcccaggtggtgtagggaaatggggcagttgccaaggctccttccagctctgggagtttaggattctcttatctcgagatttgtgggcccatgaaataatgttgttaaagcagggctagcgcatgttttctcaccatgaagtgggtcaggtagatttttttcctgtgagaatttgtgaccttttcttgaagctctgcttttaagggatatagctttgagttctgtgccccccaccctcccttctacacatacctcagcctgaccttcgccttccccctcacaggccaacaagctggcggagaagcggaaagcacacgatgaggctgtaagatcagagaagaaggcgaaaaaggcgcgacctgaggcggccgccgggggcacgggaagtggtggatcagccggtggcactggtggctctgccggagggtcagcgggagcagggggagccacaggcggatctacggctggaggggcgacaacggcctctgcgatcgctggcgaaaatctgtattttcagggaggagctagcggaagcggaatggtcagtaagggtgaggaggacaacatggctataatcaaagagtttatgcggtttaaggtccatatggaaggttcagttaatggacatgagttcgagatagaaggtgagggtgaggggcgaccgtacgaaggcacacaaaccgcaaagttgaaagtcaccaaaggtggacccttgccctttgcttgggatattctctcccctcaattcatgtacggcagtaaggcatacgtcaaacatcccgctgacatccccgactatctgaagctgtctttccctgagggttttaaatgggagcgagtgatgaacttcgaggacgggggagtggtaacagtgactcaagattcctctttgcaggacggggagttcatatataaagtgaaactgcggggtacgaactttccaagtgacggtcccgtaatgcagaagaagacgatgggatgggaggcaagcagcgagcgaatgtatcctgaggatggagcccttaagggagaaattaagcaacggctgaagttgaaagatggtggacattatgatgctgaggttaaaacaacttataaagccaagaaaccagttcagttgccaggggcgtataacgtcaacattaaactggacattacatctcacaatgaagattacacaatcgttgagcaatatgaacgcgcggagggtcggcactcaacgggtggcatggacgagttgtataaaggcgcgcccggaagcggagctactaacttcagcctgctgaagcaggctggagacgtggaggagaaccctggacctatgggctggtcatgtatcattctgtttctggtcgcaaccgcaactggagtgcattcacaggtgcagctcggcggaccgtcccaactgagccaagtaacgccagtggatgaagtggacggaaccagaacgtatcgcgttcgggggcaactctttttcgtctctacccatgacttcttgcaccagttcgactttacccatccagcaaggcgggtggtgattgacctctctgacgctcacttttgggatgggagtgccgtaggagctttggacaaggtgatgctgaagtttatgagacagggcacgagtgtcgagctgcgcgggctgaacgctgcaagtgccactcttgttgaacggcttgggagcggaactaccggtggcgaaaatctgtattttcagggagccgcggcctctaattccgctgacggtgacggttcaaatgctacagggagttctgctggtgctggctctggaacgagtggcggggacaacacgagtgatggctccggggcgagtgccggtgcagccagcacaaattcaaatgggaacacgggtagtgcgacttctgggggggccacaggtagcgatacgtcaggagcgacggctggtagtggggcttccgacggcggaaacggcgcaacagcgtcatcaactacaggcaacggaaattcaagcggtacaaccgcgacgaccggaggcggtgatgcaggggggtcgactaatgctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgttaggcgcgcaataatgccggctacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgtttcgaattaattaaatgaggcggtgaggtctgagaagaaggccaagaaggcaaggccggagtgagtgcctgcggcccctcacagggctgaggccagcccctagcagctggatgtggcagaggcaggccagaggacctaagtgtgatggaccagagtcacttctcctcctcctttctccagccagccctgacccctcatgctctctggctgggccagtgggcagccctcgcttcccttggatggagctgccctgctggtgcctggtcagagaagaggcctctgtgcccagcctgattctctgctcccaggagccagtgacatgaggtgcagaggcccacccagccccctacctactgcccccattcatcctggctttccacagccccctcccacacagttggacccgtgattctcagggtgctgtgatggggtgagggtagggggagcatttgttattaaatgactggacttttgtgccaattgcattttgtgtccatgagccttcctagggttggaggaggcctacctagcactctatgctgcaggctgggccagccctgggtatttactgagacagagctgggcactgctcagagctctctggatgtccaaggacccctccaggtccagggatgccaaaaggtaggtgcacccacgatgaggcggtgaggtct

SEQ ID NO: 16 - DNA, 4008 bp, artificial sequence
  insertion template
agttgctactgaatcgcctctggtggattggttggttagtttcaaatcttataccttaatatatgggttaagaatgaatcattctctgagtataatctaattatttttgagttacacagatgtggtggtatctttacattttttgtgtttgtgatttagatctgctactgaactttttgaggcatatagcatggcagaaatgacattcaatcctcctgttgaatcttcaaaccccaaaataaaggtatgggatatttttcatttttttaaaggaagaaatagaaaccaatgtatctcaataactctaactccagtttgcttaattattttataggtagttttttttttaatgtttaggatttcatcataggatggatttctgaggttgaaattctatagagatgatcatgaaactgttcgttcaatataggatatgtccaagaccttaccaagcatctgtcattgtgttgcatgtgttggtgtcagctgttgccattttcaacttggttcacaggttggctttagcttatagcataagtaacttctaactcatactttaaatattttcctagggtaaatttttacaaggggattcatttttgaatgaagatttaaatcagctaatcaaaagatactccagtgaagttgctactgaatcgcctcttgactttaccaagtacctcaagacaagtcttcacgcggccgccgggggcacgggaagtggtggatcagccggtggcactggtggctctgccggagggtcagcgggagcagggggagccacaggcggatctacggctggaggggcgacaacggcctctgcgatcgctttggaagtgcttttccaggggcctggagctagcggaagcggaggatcaaagggagaggaactctttaccggcgtcgttccaatccttgttgaactggatggggacgtgaatgggcataaattttcagtatcaggggaaggggaaggcgacgctacatatggaaaattgactctcaaattcatatgcactactggtaaattgcccgtgccttggcctacactcgtcacgaccttcgggtatggtgttcaatgtttcgccaggtatccggatcatatgaaacaacacgatttcttcaaatcagcgatgccggaagggtatgtgcaggagcgaacaatctttttcaaggacgacggcaactataaaacacgggccgaagtcaaatttgagggagatacgctcgttaatcggatagagctgaagggcatcgactttaaggaggatgggaacatcttgggccataagctggaatataattataacagccacaacgtttacattatggccgacaaacagaagaatggtattaaggtgaattttaaaataaggcacaacatagaagacggatctgtgcaactggccgaccactatcagcagaatacgcctattggcgatggtccagtgcttctccctgacaaccattacctcagtacgcaaagtgctctctctaaagaccccaacgaaaaacgcgatcacatggtactgctggagttcgtaaccgccgcaggaataactcatggaatggatgaactctacaaggttgacttggataaaggcgcgcccggaagttcctattctctagaaagtataggaacttcggggtctggcgaaggcagaggctcccttttgacatgcggagacgtcgaggagaacccgggtcccatggagacagacacactcctgctatgggtactgctcctctgggttccaggttccactggcgacggcggaccgaccgccaacacctcctccacctccaccaacggcaacgctgcgccacgggttattaccctttcacctgcgaacacagaattggccttcgcagcggggatcacgccggttggcgttagtagctattcagattatccgccacaggcacaaaaaatcgagcaagtctcaacttggcagggtatgaacctggaacgcatagtggctttgaagcccgacctggttatcgcttggcggggcgggaatgccgagaggcaggttgatcagttggcctccctgggtataaaagtaatgtgggtggatgcaacaagtattgaacaaatagcaaatgccttgagacagttggccccgtggagtccccagcctgacaaagctgaacaagctgctcaaagccttcttgaccagtatgcacagttgaaagcgcaatacgcagataagcctaagaagcgcgtatttttgcaatttggaattaatcctccatttacctctggtaaggagtcaattcaaaatcaagtcttggaggtctgtggaggggagaatatttttaaggatagtagggtcccctggccccaggtaagccgagaacaagtgctggcccggagtccacaggcaatcgtcatcacagggggacccgaccaaattcccaagatcaaacagtactggggggagcaactcaaaattccagtcataccactgacatcagactggttcgaacgggcaagcccccggatcatactcgctgcacaacaactctgcaatgcgttgagccaggttgacggaggaaactcctccaactccgccaccaacacctccgccaccaccggtttggaagtgcttttccaggggcctgccgcggcctctaattccgctgacggtgacggttcaaatgctacagggagttctgctggtgctggctctggaacgagtggcggggacaacacgagtgatggctccggggcgagtgccggtgcagccagcacaaattcaaatgggaacacgggtagtgcgacttctgggggggccacaggtagcgatacgtcaggagcgacggctggtagtggggcttccgacggcggaaacggcgcaacagcgtcatcaactacaggcaacggaaattcaagcggtacaaccgcgacgaccggaggcggtgatgcaggggggtcgactaatgctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgttaggcgcgcaataatggaagttcctattctctagaaagtataggaacttcgtaagccggctacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgtttcgaattaattaactctggatttcacgaaatatttgaaaacatcactacactagtagaggaatgaagtcagtggactttcttgtatatttgtgtgtgcagatgtacataaagatgagttgttaacttaggatcttttctttttatacaaggaaagcttcctaagaatgtctaggaagaagaggaagaatgaccctttgcatggcacagggttctgcccctattctgaatatgtcattccatcaaggagatcaaaagcctttttttctccccagtatttggaaattactttcttgatgatgctgccttttaaaagcttcacgtacattatagttttttaaaaaaatctttggactggatcttactgaagtgcagttgctatattaaaattagggcatagagcacagaaaaatcaagaccatgagaagacattttaccatttagctactttttataactaaatactctttaaatatttttatttcaatactgtggatggaaatgagaagcattctaaatttgagttaatatatttttatgaagatatttgagaaaagaaaaaaatagcttgtattcaggttcattggcttttgctggatgatccacctaaagaagttacctaatttggccttttaagttgctactgaatcgcctctgg

SEQ ID NO: 17 - DNA, 3738 bp, artificial sequence
  insertion template
agttgctactgaatcgcctctggtggattggttggttagtttcaaatcttataccttaatatatgggttaagaatgaatcattctctgagtataatctaattatttttgagttacacagatgtggtggtatctttacattttttgtgtttgtgatttagatctgctactgaactttttgaggcatatagcatggcagaaatgacattcaatcctcctgttgaatcttcaaaccccaaaataaaggtatgggatatttttcatttttttaaaggaagaaatagaaaccaatgtatctcaataactctaactccagtttgcttaattattttataggtagttttttttttaatgtttaggatttcatcataggatggatttctgaggttgaaattctatagagatgatcatgaaactgttcgttcaatataggatatgtccaagaccttaccaagcatctgtcattgtgttgcatgtgttggtgtcagctgttgccattttcaacttggttcacaggttggctttagcttatagcataagtaacttctaactcatactttaaatattttcctagggtaaatttttacaaggggattcatttttgaatgaagatttaaatcagctaatcaaaagatactccagtgaagttgctactgaatcgcctcttgactttaccaagtacctcaagacaagtcttcacgcggccgccgggggcacgggaagtggtggatcagccggtggcactggtggctctgccggagggtcagcgggagcagggggagccacaggcggatctacggctggaggggcgacaacggcctctgcgatcgctttggaagtgcttttccaggggcctggagctagcggaagcggaggatcaaagggagaggaactctttaccggcgtcgttccaatccttgttgaactggatggggacgtgaatgggcataaattttcagtatcaggggaaggggaaggcgacgctacatatggaaaattgactctcaaattcatatgcactactggtaaattgcccgtgccttggcctacactcgtcacgaccttcgggtatggtgttcaatgtttcgccaggtatccggatcatatgaaacaacacgatttcttcaaatcagcgatgccggaagggtatgtgcaggagcgaacaatctttttcaaggacgacggcaactataaaacacgggccgaagtcaaatttgagggagatacgctcgttaatcggatagagctgaagggcatcgactttaaggaggatgggaacatcttgggccataagctggaatataattataacagccacaacgtttacattatggccgacaaacagaagaatggtattaaggtgaattttaaaataaggcacaacatagaagacggatctgtgcaactggccgaccactatcagcagaatacgcctattggcgatggtccagtgcttctccctgacaaccattacctcagtacgcaaagtgctctctctaaagaccccaacgaaaaacgcgatcacatggtactgctggagttcgtaaccgccgcaggaataactcatggaatggatgaactctacaaggttgacttggataaaggcgcgcccggaagttcctattctctagaaagtataggaacttcggggtctggcgaaggcagaggctcccttttgacatgcggagacgtcgaggagaacccgggtcccatggagacagacacactcctgctatgggtactgctcctctgggttccaggttccactggcgacggcggaccgaccgccaacacctcctccacctccaccaacggcaacagcattttggacatacgccaaggcccgaaagagccatttcgcgattacgtagatcggttctacaaaacgctgcgagcggagcaagcatcacaagaggttaaaaattggatgacggagacattgcttgttcaaaacgcgaacccagattgtaaaacaattttgaaagcccttggacctggtgctacgctcgaggaaatgatgacagcatgccaaggcgttggtggaccaggaggaagtaccggaggaagcatccttgatatacgacaaggtcctaaggagccttttcgcgactacgttgaccgcttttataagacgcttcgcgctgaacaggcgtctcaggaggtcaagaattggatgacagagacattgcttgtacaaaatgctaatcccgactgtaaaacgattctcaaggcgctgggaccgggagccactcttgaagaaatgatgactgcgtgtcaaggagtaggaggaaactcctccaactccgccaccaacacctccgccaccaccggtggcgaaaatctgtattttcagggagccgcggcctctaattccgctgacggtgacggttcaaatgctacagggagttctgctggtgctggctctggaacgagtggcggggacaacacgagtgatggctccggggcgagtgccggtgcagccagcacaaattcaaatgggaacacgggtagtgcgacttctgggggggccacaggtagcgatacgtcaggagcgacggctggtagtggggcttccgacggcggaaacggcgcaacagcgtcatcaactacaggcaacggaaattcaagcggtacaaccgcgacgaccggaggcggtgatgcaggggggtcgactaatgctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgttaggcgcgcaataatggaagttcctattctctagaaagtataggaacttcgtaagccggctacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgtttcgaattaattaactctggatttcacgaaatatttgaaaacatcactacactagtagaggaatgaagtcagtggactttcttgtatatttgtgtgtgcagatgtacataaagatgagttgttaacttaggatcttttctttttatacaaggaaagcttcctaagaatgtctaggaagaagaggaagaatgaccctttgcatggcacagggttctgcccctattctgaatatgtcattccatcaaggagatcaaaagcctttttttctccccagtatttggaaattactttcttgatgatgctgccttttaaaagcttcacgtacattatagttttttaaaaaaatctttggactggatcttactgaagtgcagttgctatattaaaattagggcatagagcacagaaaaatcaagaccatgagaagacattttaccatttagctactttttataactaaatactctttaaatatttttatttcaatactgtggatggaaatgagaagcattctaaatttgagttaatatatttttatgaagatatttgagaaaagaaaaaaatagcttgtattcaggttcattggcttttgctggatgatccacctaaagaagttacctaatttggccttttaagttgctactgaatcgcctctgg

SEQ ID NO: 18 - DNA, 3844 bp, artificial sequence
  insertion template
acctaccacttcccatgttggggcctccaaaaactcactacttaagactaattggatcaaagtgtttaccagttggaaaaatcttgcataagtctgcattataaaatgtgtttaaagaattacaatttaattatttttatgtatatacgtaagctcttactgcctaagaattctttccaaatataaggcctagggctacttgaataatttgtaatatacaattaatgtgttgtcctttaaaaatttttaattttctttaataggtaaaactgtatccctttcaaacttatgtatcttggcagatgctttatagaaagtgcaacagcatattatgtctcaaccaaatttaaatgatagcttttaatgttttaataaactgtatcatagtatagtagtgaaacaacgttggtccctttactcactctcaatgcaagttaactgctcacccataattccttttgtaatgaaaatcattagtatttaattaggtttagctatgatgtgaaataattatatttatttatgttttcttgtctttttctctccttttacacagctacttctgagtggaggagcagacaataaattgtattcctacagatattcacctaccacttcccatgttggtgcagcggccgccggaggtactggatcaggtggatcagcaggaggcggtactggaggttctgctggcggttcagctggtgcgggcgcgacgggtggaagtacagccggaggtgccacgacagcgtcccatcaccaccatcaccatcatcatcatcattatccatatgacgtacctgattatgcggcgatcgctggcgagaacctgtattttcaagggagctcgagtccttcaagacttgaggaagaattgagacggagacttaccgagcccggcgcacagagtggtttggaggtgcttttccagggaccaggtgctagcggaagcggaatggtcagtaagggtgaggaggacaacatggctataatcaaagagtttatgcggtttaaggtccatatggaaggttcagttaatggacatgagttcgagatagaaggtgagggtgaggggcgaccgtacgaaggcacacaaaccgcaaagttgaaagtcaccaaaggtggacccttgccctttgcttgggatattctctcccctcaattcatgtacggcagtaaggcatacgtcaaacatcccgctgacatccccgactatctgaagctgtctttccctgagggttttaaatgggagcgagtgatgaacttcgaggacgggggagtggtaacagtgactcaagattcctctttgcaggacggggagttcatatataaagtgaaactgcggggtacgaactttccaagtgacggtcccgtaatgcagaagaagacgatgggatgggaggcaagcagcgagcgaatgtatcctgaggatggagcccttaagggagaaattaagcaacggctgaagttgaaagatggtggacattatgatgctgaggttaaaacaacttataaagccaagaaaccagttcagttgccaggggcgtataacgtcaacattaaactggacattacatctcacaatgaagattacacaatcgttgagcaatatgaacgcgcggagggtcggcactcaacgggtggcatggacgagttgtataaaggcgcgcccataacttcgtatagcatacattatacgaagttatctgggtctggcgaaggcagaggctcccttttgacatgcggagacgtcgaggagaacccgggtcccatggagacagacacactcctgctatgggtactgctcctctgggttccaggttccactggcgacggcggaccgtctaacacagcaaatgggactagcaccacgaacgcatatccttacgacgttcctgattacgcttcatctggtggaagtggcaccggagggacttatccgtacgacgtacctgactatgcttccacaagcggggggactggtggtggcagttatccctacgacgttcccgattatgcgggcacaggttccgggagtactggtggctcctatccttatgatgtccccgattatgcgtccagcggcggcggctctactacagggggttatccctatgatgttccagattacgccacttcaggttccgggactggatctggaggatacccttatgatgtaccagattacgctactagtggctctggcacaggaggcggttcatacccctacgatgttccggactacgcgggatctgggagcggcagcacgaccagtggttatccctatgacgttccagactacgccgggacgggaacagggagttcctccggcgggtatccatatgacgtaccagattatgcgacctctagcggaaccgggggttctggagggtatccgtatgacgtgcctgactacgccaatactacatctaacactagtgcatccgcgaatagtaccggtggcgaaaatctgtattttcagggagccgcggcctctaattccgctgacggtgacggttcaaatgctacagggagttctgctggtgctggctctggaacgagtggcggggacaacacgagtgatggctccggggcgagtgccggtgcagccagcacaaattcaaatgggaacacgggtagtgcgacttctgggggggccacaggtagcgatacgtcaggagcgacggctggtagtggggcttccgacggcggaaacggcgcaacagcgtcatcaactacaggcaacggaaattcaagcggtacaaccgcgacgaccggaggcggtgatgcaggggggtcgactaatgctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgttaggcgcgcaataatataacttcgtatagcatacattatacgaagttataagccggctacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgtttcgaattaattaatgaaagtgaacaataatttgactatagagattatttctgtaaatgaaattggtagagaaccatgaaattacatagatgcagatgcagaaagcagccttttgaagtttatataatgttttcacccttcataacagctaacgtatcactttttcttattttgtatttataataagataggttgtgtttataaaatacaaactgtggcatacattctctatacaaacttgaaattaaactgagttttacatttctctttaaaggtattggtttgaattcagatttgcttttttatttttatttgttttttttttttttgagatggagtcttgctctgttgcctaggctggagtgcagtggcgcaatctcaactcactgcaacctccgcttcctaggttcaatcgattctcctgtctcaacctcccaagtagctgggattacaggcacacatcacgatgtcctgctaatttttgtatttttagtagagacggggttttgccatgttggccaggctggtcttgaactcctgacctcaggtgatctgcccacctcagcctcccaaagtgagccactgtgcctggccgaattaagatttgtttttacctaccacttcccatgttgggg

SEQ ID NO: 19 - DNA, 3625 bp, artificial sequence
  insertion template
acctaccacttcccatgttggggcctccaaaaactcactacttaagactaattggatcaaagtgtttaccagttggaaaaatcttgcataagtctgcattataaaatgtgtttaaagaattacaatttaattatttttatgtatatacgtaagctcttactgcctaagaattctttccaaatataaggcctagggctacttgaataatttgtaatatacaattaatgtgttgtcctttaaaaatttttaattttctttaataggtaaaactgtatccctttcaaacttatgtatcttggcagatgctttatagaaagtgcaacagcatattatgtctcaaccaaatttaaatgatagcttttaatgttttaataaactgtatcatagtatagtagtgaaacaacgttggtccctttactcactctcaatgcaagttaactgctcacccataattccttttgtaatgaaaatcattagtatttaattaggtttagctatgatgtgaaataattatatttatttatgttttcttgtctttttctctccttttacacagctacttctgagtggaggagcagacaataaattgtattcctacagatattcacctaccacttcccatgttggtgcagcggccgccggaggtactggatcaggtggatcagcaggaggcggtactggaggttctgctggcggttcagctggtgcgggcgcgacgggtggaagtacagccggaggtgccacgacagcgtcccatcaccaccatcaccatcatcatcatcattatccatatgacgtacctgattatgcggcgatcgctggcgagaacctgtattttcaagggagctcgagtccttcaagacttgaggaagaattgagacggagacttaccgagcccggcgcacagagtggtttggaggtgcttttccagggaccaggtgctagcggaagcggaatggtcagtaagggtgaggaggacaacatggctataatcaaagagtttatgcggtttaaggtccatatggaaggttcagttaatggacatgagttcgagatagaaggtgagggtgaggggcgaccgtacgaaggcacacaaaccgcaaagttgaaagtcaccaaaggtggacccttgccctttgcttgggatattctctcccctcaattcatgtacggcagtaaggcatacgtcaaacatcccgctgacatccccgactatctgaagctgtctttccctgagggttttaaatgggagcgagtgatgaacttcgaggacgggggagtggtaacagtgactcaagattcctctttgcaggacggggagttcatatataaagtgaaactgcggggtacgaactttccaagtgacggtcccgtaatgcagaagaagacgatgggatgggaggcaagcagcgagcgaatgtatcctgaggatggagcccttaagggagaaattaagcaacggctgaagttgaaagatggtggacattatgatgctgaggttaaaacaacttataaagccaagaaaccagttcagttgccaggggcgtataacgtcaacattaaactggacattacatctcacaatgaagattacacaatcgttgagcaatatgaacgcgcggagggtcggcactcaacgggtggcatggacgagttgtataaaggcgcgcccataacttcgtatagcatacattatacgaagttatctgggtctggcgaaggcagaggctcccttttgacatgcggagacgtcgaggagaacccgggtcccatggagacagacacactcctgctatgggtactgctcctctgggttccaggttccactggcgacggcggaccgtctaacacagcaaatgggactagcaccacgaacgcagactacaaggacgacgacgataagaccggcagcgattataaggatgatgacgataagagttccggcgactataaggacgacgatgataaggggaccactgattacaaagacgatgacgacaaaggcgggtccgactataaggatgacgatgacaagagcggaagtgattacaaagatgatgacgacaagaccgggactgattataaagatgatgatgataaaggctccagtgattataaagacgacgacgacaagggcagtggagactacaaagacgacgatgacaagggtactggcgattacaaggatgatgatgacaagaatactacatctaacactagtgcatccgcgaatagtaccggtggcgaaaatctgtattttcagggagccgcggcctctaattccgctgacggtgacggttcaaatgctacagggagttctgctggtgctggctctggaacgagtggcggggacaacacgagtgatggctccggggcgagtgccggtgcagccagcacaaattcaaatgggaacacgggtagtgcgacttctgggggggccacaggtagcgatacgtcaggagcgacggctggtagtggggcttccgacggcggaaacggcgcaacagcgtcatcaactacaggcaacggaaattcaagcggtacaaccgcgacgaccggaggcggtgatgcaggggggtcgactaatgctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgttaggcgcgcaataatataacttcgtatagcatacattatacgaagttataagccggctacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgtttcgaattaattaatgaaagtgaacaataatttgactatagagattatttctgtaaatgaaattggtagagaaccatgaaattacatagatgcagatgcagaaagcagccttttgaagtttatataatgttttcacccttcataacagctaacgtatcactttttcttattttgtatttataataagataggttgtgtttataaaatacaaactgtggcatacattctctatacaaacttgaaattaaactgagttttacatttctctttaaaggtattggtttgaattcagatttgcttttttatttttatttgttttttttttttttgagatggagtcttgctctgttgcctaggctggagtgcagtggcgcaatctcaactcactgcaacctccgcttcctaggttcaatcgattctcctgtctcaacctcccaagtagctgggattacaggcacacatcacgatgtcctgctaatttttgtatttttagtagagacggggttttgccatgttggccaggctggtcttgaactcctgacctcaggtgatctgcccacctcagcctcccaaagtgagccactgtgcctggccgaattaagatttgtttttacctaccacttcccatgttgggg

SEQ ID NO: 20 - RNA, 20 nt, artificial sequence
  sgRNA sequence
gucccaccugggaaccucgc

SEQ ID NO: 21 - RNA, 20 nt, artificial sequence
  sgRNA sequence
agaccucaccgccucaucgu

SEQ ID NO: 22 - RNA, 20 nt, artificial sequence
  sgRNA sequence
aguugcuacugaaucgccuc

SEQ ID NO: 23 - RNA, 20 nt, artificial sequence
  sgRNA sequence
accuaccacuucccauguug

SEQ ID NO: 24 - DNA, 2064 bp, artificial sequence
  modified recombinase sequence
atgccacaatttgatatattatgtaaaacaccacctaaggtgcttgttcgtcagtttgtggaaaggtttgaaagaccttcaggtgagaaaatagcattatgtgctgctgaactaacctatttatgttggatgattacacataacggaacagcaatcaagagagccacattcatgagctataatactatcataagcaattcgctgagtttggatattgtcaacaagtcactgcagtttaaatacaagacgcaaaaagcaacaattctggaagcctcattaaagaaattgatacctgcttgggaatttacaattattccttactatggacaaaaacatcaatctgatatcactgatattgtaagtagtttgcaattacagttcgaatcatcggaagaagcagataagggaaatagccacagtaaaaaaatgcttaaagcacttctaagtgagggtgaaagcatctgggagatcactgagaaaatactaaattcgtttgagtatacttcgagatttacaaaaacaaaaactttataccaattcctcttcctagctactttcatcaattgtggaagattcagcgatattaagaacgttgatccgaaatcatttaaattagtccaaaataagtatctgggagtaataatccagtgtttagtgacagagacaaagacaagcgttagtaggcacatatacttctttagcgcaaggggtaggatcgatccacttgtatatttggatgaatttttgaggaattctgaaccagtcctaaaacgagtaaataggaccggcaattcttcaagcaacaaacaggaataccaattattaaaagataacttagtcagatcgtacaacaaagctttgaagaaaaatgcgccttattcaatctttgctataaaaaatggcccaaaatctcacattggaagacatttgatgacctcatttctttcaatgaagggcctaacggagttgactaatgttgtgggaaattggagcgataagcgtgcttctgccgtggccaggacaacgtatactcatcagataacagcaatacctgatcactacttcgctctagtttctcggtactatgcttatgatccaatatcaaaggaaatgatagcattgaaggatgagactaatccaattgaggagtggcagcatatagaacagctaaagggtagtgctgaaggaagcatacgataccccgcatggaatgggataatatcacaggaggtactagactacctttcatcctacataaatagacgcatagcggccgccggaagcggagccactaacttctccctgttgaaacaagcaggggatgtcgaagagaatcccgggccaaccggtggcgcgcctggtatgagcgagctgattaaggagaacatgcacatgaagctgtacatggagggcaccgtggacaaccatcacttcaagtgcacatccgagggcgaaggcaagccttacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacgccaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctgcctagcaaactggggcacaaacttaattaa